(19) United States
(12) Patent Application Publication
(10) Pub. No.: US 2024/0341637 A1
(43) Pub. Date: Oct. 17, 2024

(54) APPARATUSES, SYSTEMS, AND METHODS FOR PATCH APPLICATORS FOR MEDICAL DEVICES

(71) Applicant: DexCom, Inc., San Diego, CA (US)

(72) Inventors: Mark Selander, San Diego, CA (US); Young Woo Lee, San Diego, CA (US)

(21) Appl. No.: 18/634,724

(22) Filed: Apr. 12, 2024

Related U.S. Application Data
(60) Provisional application No. 63/459,382, filed on Apr. 14, 2023.

(51) Int. Cl. A61B 5/145; A61B 5/00

(52) U.S. Cl. CPC A61B 5/14532; A61B 5/0002; A61B 5/14503; A61B 5/6833; A61B 5/68335; A61B 2560/045

(57) ABSTRACT

The present examples relate generally to patch applicators for medical devices. The medical devices may comprise a transcutaneous analyte sensor applied to the skin of a host. The apparatuses, systems, and methods disclosed may be directed to an applicator for an overlay patch for being applied to at least a portion of an on skin medical device.

### CROSS REFERENCE TO RELATED APPLICATIONS

The present application claims the benefit of U.S. Provisional Patent Application No. 63/459,382, filed Apr. 14, 2023, the entire contents of which are incorporated herein by reference.

### BACKGROUND OF THE INVENTION

### Field of the Invention

Medical device apparatuses, systems, and methods. More particularly, apparatuses, systems, and methods are provided for patch applicators for medical devices, which may include a transcutaneous analyte sensor for deployment into the skin of a host.

### Description of the Related Technology

Diabetes mellitus is a disorder in which the pancreas cannot create sufficient insulin (Type 1 or insulin dependent) and/or in which insulin is not effective (Type 2 or non-insulin dependent). In the diabetic state, the victim suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Conventionally, a person with diabetes carries a self-monitoring blood glucose (SMBG) monitor, which typically requires uncomfortable finger pricking methods. Due to the lack of comfort and convenience, a person with diabetes normally only measures his or her glucose levels two to four times per day. Unfortunately, such time intervals are spread so far apart that the person with diabetes likely finds out too late of a hyperglycemic or hypoglycemic condition, sometimes incurring dangerous side effects. Glucose levels may be alternatively monitored continuously by a measurement system including an on-skin sensor assembly. The sensor assembly may have a wireless transmitter which transmits measurement data to a receiver which can process and display information based on the measurements.

An applicator may be utilized to deploy an on-skin sensor assembly to a person. The application process should result in the on-skin sensor assembly being attached to the person in a state where it is capable of sensing the analyte (e.g., glucose) level information, communicating the sensed data to the transmitter, and transmitting the analyte level information to the receiver.

Exemplary systems are disclosed in, e.g., U.S. Patent Publication No. 2014/0088389, U.S. Patent Publication No. 2013/0267813, and U.S. Patent Publication No. 2018/0368771, owned by the assignee of the present application and herein incorporated by reference in their entireties.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

The present apparatuses, systems, and methods relate to apparatuses, systems, and methods for medical devices. More particularly, apparatuses, systems, and methods are provided for patch applicators for medical devices, which may include a transcutaneous analyte sensor for deployment to the skin of a host. The various examples of the present apparatuses, systems, and methods may have several features, no single one of which is solely responsible for their desirable attributes. Without limiting the scope of the present examples as expressed by the claims that follow, their more prominent features now will be discussed briefly. After considering this discussion, and particularly after reading the section entitled “Detailed Description,” one will understand how the features of the present examples provide the advantages described herein.

In a first aspect, a system for applying a patch onto at least a portion of an on-skin wearable medical device of a host, the system comprising: an applicator for supporting the patch and configured to apply the patch onto at least the portion of the on-skin wearable medical device when the on-skin wearable medical device is disposed on a skin of the host.

Implementations of the embodiments may include one or more of the following. The applicator may include a substrate for supporting the patch. The substrate may comprise a planar surface for the patch to be positioned upon and extend parallel with the planar surface. The substrate is flexible. The substrate may be made from one or more of a polymer, a metal, or a paper-based material. The applicator may include an alignment body configured to align the patch with the on-skin wearable medical device. The alignment body may comprise a protrusion configured to extend towards the skin of the host upon the applicator applying the patch onto at least the portion of the on-skin wearable medical device. The protrusion may include a central opening configured to receive a portion of the on-skin wearable medical device. The central opening may be configured to receive a housing of the on-skin wearable medical device. The applicator may include a substrate for supporting the patch, the substrate having a central portion, and the protrusion configured to extend from the central portion of the substrate. The applicator may include a substrate for supporting the patch, the protrusion configured to extend from the substrate and configured to displace relative to the substrate. The protrusion may be configured to retract from the skin of the host upon the applicator applying the patch onto at least the portion of the on-skin wearable medical device. The applicator may include a spring configured to bias the protrusion relative to the substrate. The spring may be configured to bias the protrusion to be raised relative to the substrate. The spring may comprise a coil spring. The spring may comprise a flat spring. The applicator may include a plunger configured to be operated by a user to displace the protrusion relative to the substrate. The applicator may include a substrate for supporting the patch, the substrate comprising a disk, and the protrusion comprises a cylinder extending from the disk. The protrusion may include a leading edge configured to face towards the skin of the host upon the applicator applying the patch onto at least the portion of the on-skin wearable medical device, the leading edge having a varied height. The leading edge may have an undulating height. The protrusion may be configured to be formed by folding material from a flattened configuration to a raised configuration. The alignment body may include an opening configured to receive a portion of the on-skin wearable medical device. The applicator may include a first housing and a second housing, the first housing configured to slide relative to the second housing along an axis to apply the patch onto at least the portion of the on-skin wearable medical device, and the second housing including a cavity with a coil spring disposed therein, the coil spring biasing the first housing relative to the second housing and extending circumferentially about the axis. The second housing may include a substrate for supporting the patch, and the first housing includes an alignment body configured to align the patch with the on-skin wearable medical device. The first housing may include a substrate for supporting the patch and includes an alignment body configured to align the patch with the on-skin wearable medical device. The applicator may include a ridge for supporting the patch. The ridge may be configured to engage the patch, and the ridge is configured to disengage from the patch upon application of the patch onto at least the portion of the on-skin wearable medical device. The applicator may include a substrate for supporting an interior annular section of a patch assembly including one or more of the patch, a liner for the patch, or a backing layer for the patch. The substrate may comprise a ridge. The substrate may be configured to engage the interior annular section, and the applicator includes a pressure surface for pressing at least a portion of the patch assembly towards the skin of the host to disengage the interior annular section from the substrate. The pressure surface may have an annular shape and is configured to press the portion of the patch assembly at a location radially outward of the substrate. The applicator may include an alignment body configured to align the patch with the on-skin wearable medical device, the alignment body having a leading edge, and the substrate being positioned at the leading edge. The applicator may include an alignment body configured to align the patch with the on-skin wearable medical device, the alignment body having a leading edge, and the substrate being spaced from the leading edge. The substrate may comprise a datum feature for setting a distance of the patch from the leading edge prior to application of the patch onto at least the portion of the on-skin wearable medical device. The patch may be bonded to the applicator and extends radially outward from the applicator. The applicator may be configured to be dispensed onto the on-skin wearable medical device upon application of the patch. The applicator may include an alignment body configured to align the patch with the on-skin wearable medical device. The alignment body may comprise a shell bounding a cavity, the cavity adapted to house a housing of the on-skin wearable medical device therein. An adhesive may be positioned within the cavity and configured to adhere the shell to the housing of the on-skin wearable medical device. The shell may comprise a molded shell and the patch extends radially outward from the molded shell. The applicator may include a displacement portion configured to allow a first portion of the applicator to displace relative to a second portion of the applicator. The first portion of the applicator may comprise a substrate for supporting the patch, and the second portion of the applicator comprises a central portion of the applicator. The first portion of the applicator may comprise a substrate for supporting the patch, and the second portion of the applicator comprises an alignment body configured to align the patch with the on-skin wearable medical device. The applicator may include a substrate for supporting the patch, and the displacement portion is more flexible than the substrate. The applicator may include a substrate for supporting the patch, and the displacement portion includes one or more voids in the substrate. The one or more voids may form one or more arcs in the substrate. The applicator may include a substrate for supporting the patch, and the displacement portion includes one or more connectors in the substrate. The displacement portion may comprise a plurality of voids and connectors. The displacement portion may comprise a spring. The spring may be formed by a plurality of voids and connectors. The applicator may include one or more retainers configured to retain the patch to the applicator. The applicator may include a substrate for supporting the patch, and the one or more retainers comprise one or more slots in the substrate. The applicator may include a liner removal assembly configured to remove a liner from the patch. The liner removal assembly may be configured to couple to the liner of the patch and automatically remove the liner from the patch during an application procedure of the patch onto at least the portion of the on-skin wearable medical device. The applicator may comprise packaging for the on-skin wearable medical device. The applicator may comprise a cap for an applicator housing of the on-skin wearable medical device. The applicator may be configured to retain a plurality of patches. The plurality of patches may include a first patch and a second patch, and the applicator is configured to apply the first patch to at least the portion of the on-skin wearable medical device and automatically position the second patch for application to at least a portion of an on-skin wearable medical device upon application of the first patch. The patch may include an adhesive surface for adhering to at least the portion of the on-skin wearable medical device. A liner may cover the adhesive surface. The patch may have a ring shape. The patch may include a central opening for receiving a portion of the on-skin wearable medical device. A backing layer may be coupled to the patch, the backing layer having one or more cuts or perforations configured to allow for disengagement of the backing layer from the applicator. The patch may be an overlay patch, and further comprising the on-skin wearable medical device including an on-skin wearable housing and an underlay patch, the underlay patch extending radially outward from the on-skin wearable housing. The underlay patch may have a first diameter, and the overlay patch has a second diameter that is greater than the first diameter. The underlay patch may include an adhesive surface for adhering to the skin of the host, and includes an upper surface facing opposite the adhesive surface, the upper surface configured for the overlay patch to be disposed upon. The on-skin wearable medical device may include a transcutaneous analyte sensor. The on-skin wearable medical device may include a continuous glucose monitoring system. The patch may be a first patch configured to be applied to a second patch. The first patch may be an overlay patch and the second patch may be an underlay patch.

In a second aspect, a method comprising utilizing an applicator to apply a patch onto at least a portion of an on-skin wearable medical device when the on-skin wearable medical device is disposed on a skin of a host.

Implementations of the embodiments may include one or more of the following. The applicator may include a substrate for supporting the patch. The substrate may comprise a planar surface for the patch to be positioned upon and extend parallel with the planar surface. The substrate is flexible. The substrate may be made from one or more of a polymer, a metal, or a paper-based material. The applicator may include an alignment body configured to align the patch with the on-skin wearable medical device. The alignment body may comprise a protrusion configured to extend towards the skin of the host upon the applicator applying the patch onto at least the portion of the on-skin wearable medical device. The protrusion may include a central opening configured to receive a portion of the on-skin wearable medical device. The central opening may be configured to receive a housing of the on-skin wearable medical device. The applicator may include a substrate for supporting the patch, the substrate having a central portion, and the protrusion configured to extend from the central portion of the substrate. The applicator may include a substrate for supporting the patch, the protrusion configured to extend from the substrate and configured to displace relative to the substrate. The protrusion may be configured to retract from the skin of the host upon the applicator applying the patch onto at least the portion of the on-skin wearable medical device. The applicator may include a spring configured to bias the protrusion relative to the substrate. The spring may be configured to bias the protrusion to be raised relative to the substrate. The spring may comprise a coil spring. The spring may comprise a flat spring. The applicator may include a plunger configured to be operated by a user to displace the protrusion relative to the substrate. The applicator may include a substrate for supporting the patch, the substrate comprising a disk, and the protrusion comprises a cylinder extending from the disk. The protrusion may include a leading edge configured to face towards the skin of the host upon the applicator applying the patch onto at least the portion of the on-skin wearable medical device, the leading edge having a varied height. The leading edge may have an undulating height. The protrusion may be configured to be formed by folding material from a flattened configuration to a raised configuration. The alignment body may include an opening configured to receive a portion of the on-skin wearable medical device. The applicator may include a first housing and a second housing, the first housing configured to slide relative to the second housing along an axis to apply the patch onto at least the portion of the on-skin wearable medical device, and the second housing including a cavity with a coil spring disposed therein, the coil spring biasing the first housing relative to the second housing and extending circumferentially about the axis. The second housing may include a substrate for supporting the patch, and the first housing includes an alignment body configured to align the patch with the on-skin wearable medical device. The first housing may include a substrate for supporting the patch and includes an alignment body configured to align the patch with the on-skin wearable medical device. The applicator may include a ridge for supporting the patch. The ridge may be configured to engage the patch, and the ridge is configured to disengage from the patch upon application of the patch onto at least the portion of the on-skin wearable medical device. The applicator may include a substrate for supporting an interior annular section of a patch assembly including one or more of the patch, a liner for the patch, or a backing layer for the patch. The substrate may comprise a ridge. The substrate may be configured to engage the interior annular section, and the applicator includes a pressure surface for pressing at least a portion of the patch assembly towards the skin of the host to disengage the interior annular section from the substrate. The pressure surface may have an annular shape and is configured to press the portion of the patch assembly at a location radially outward of the substrate. The applicator may include an alignment body configured to align the patch with the on-skin wearable medical device, the alignment body having a leading edge, and the substrate being positioned at the leading edge. The applicator may include an alignment body configured to align the patch with the on-skin wearable medical device, the alignment body having a leading edge, and the substrate being spaced from the leading edge. The substrate may comprise a datum feature for setting a distance of the patch from the leading edge prior to application of the patch onto at least the portion of the on-skin wearable medical device. The patch may be bonded to the applicator and extends radially outward from the applicator. The applicator may be configured to be dispensed onto the on-skin wearable medical device upon application of the patch. The applicator may include an alignment body configured to align the patch with the on-skin wearable medical device. The alignment body may comprise a shell bounding a cavity, the cavity adapted to house a housing of the on-skin wearable medical device therein. An adhesive may be positioned within the cavity and configured to adhere the shell to the housing of the on-skin wearable medical device. The shell may comprise a molded shell and the patch extends radially outward from the molded shell. The applicator may include a displacement portion configured to allow a first portion of the applicator to displace relative to a second portion of the applicator. The first portion of the applicator may comprise a substrate for supporting the patch, and the second portion of the applicator comprises a central portion of the applicator. The first portion of the applicator may comprise a substrate for supporting the patch, and the second portion of the applicator comprises an alignment body configured to align the patch with the on-skin wearable medical device. The applicator may include a substrate for supporting the patch, and the displacement portion is more flexible than the substrate. The applicator may include a substrate for supporting the patch, and the displacement portion includes one or more voids in the substrate. The one or more voids may form one or more arcs in the substrate. The applicator may include a substrate for supporting the patch, and the displacement portion includes one or more connectors in the substrate. The displacement portion may comprise a plurality of voids and connectors. The displacement portion may comprise a spring. The spring may be formed by a plurality of voids and connectors. The applicator may include one or more retainers configured to retain the patch to the applicator. The applicator may include a substrate for supporting the patch, and the one or more retainers comprise one or more slots in the substrate. The applicator may include a liner removal assembly configured to remove a liner from the patch. The liner removal assembly may be configured to couple to the liner of the patch and automatically remove the liner from the patch during an application procedure of the patch onto at least the portion of the on-skin wearable medical device. The applicator may comprise packaging for the on-skin wearable medical device. The applicator may comprise a cap for an applicator housing of the on-skin wearable medical device. The applicator may be configured to retain a plurality of patches. The plurality of patches may include a first patch and a second patch, and the applicator is configured to apply the first patch to at least the portion of the on-skin wearable medical device and automatically position the second patch for application to at least a portion of an on-skin wearable medical device upon application of the first patch. The patch may include an adhesive surface for adhering to at least the portion of the on-skin wearable medical device. A liner may cover the adhesive surface. The patch may have a ring shape. The patch may include a central opening for receiving a portion of the on-skin wearable medical device. A backing layer may be coupled to the patch, the backing layer having one or more cuts or perforations configured to allow for disengagement of the backing layer from the applicator. The patch may be an overlay patch, and further comprising the on-skin wearable medical device including an on-skin wearable housing and an underlay patch, the underlay patch extending radially outward from the on-skin wearable housing. The underlay patch may have a first diameter, and the overlay patch has a second diameter that is greater than the first diameter. The underlay patch may include an adhesive surface for adhering to the skin of the host, and includes an upper surface facing opposite the adhesive surface, the upper surface configured for the overlay patch to be disposed upon. The on-skin wearable medical device may include a transcutaneous analyte sensor. The on-skin wearable medical device may include a continuous glucose monitoring system. The patch may be a first patch configured to be applied to a second patch. The first patch may be an overlay patch and the second patch may be an underlay patch.

Any of the features of an embodiment of any of the aspects, including but not limited to any embodiments of any of the first through second aspects referred to above, is applicable to all other aspects and embodiments identified herein, including but not limited to any embodiments of any of the first through second aspects referred to above. Moreover, any of the features of an embodiment of the various aspects, including but not limited to any embodiments of any of the first through second aspects referred to above, is independently combinable, partly or wholly with other embodiments described herein in any way, e.g., one, two, or three or more embodiments may be combinable in whole or in part. Further, any of the features of an embodiment of the various aspects, including but not limited to any embodiments of any of the first through second aspects referred to above, may be made optional to other aspects or embodiments. Any aspect or embodiment of a method can be performed by a system or apparatus of another aspect or embodiment, and any aspect or embodiment of a system or apparatus can be configured to perform a method of another aspect or embodiment, including but not limited to any embodiments of any of the first through second aspects referred to above.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar examples.

FIG. 1 illustrates a schematic view of a continuous analyte sensor system.

FIG. 2A illustrates a top perspective assembly view of an on-skin sensor assembly.

FIG. 2B illustrates a bottom perspective view of the on-skin sensor assembly of FIG. 2A in an assembled state.

FIG. 2C illustrates a top perspective view of the on-skin sensor assembly of FIG. 2A in an assembled state.

FIG. 3 illustrates a perspective assembly view of an on-skin sensor assembly.

FIG. 4 illustrates a perspective view of an on-skin sensor assembly.

FIG. 5 illustrates a perspective view of an applicator system for an on-skin sensor assembly of an analyte sensor system.

FIG. 6 illustrates an exploded perspective view of the applicator system of FIG. 5.

FIGS. 7-9 illustrate several cross-sectional views of the applicator system of FIGS. 5 and 6, taken along the section line A-A′ of FIG. 5, during operation.

FIGS. 10-12 illustrate several cross-sectional views of the applicator system of FIGS. 5 and 6, taken along the section line B-B′ of FIG. 5, during operation.

FIGS. 13 and 14 illustrate magnified views of some features of the applicator system of FIGS. 5 and 6.

FIGS. 15 and 16 illustrate magnified views of some features of the applicator system of FIGS. 5 and 6.

FIG. 17 illustrates a perspective partial cutaway view of the needle carrier assembly, hub, and on-skin sensor assembly of the applicator system of FIGS. 5 and 6.

FIG. 18 illustrates a cross-sectional view of the hub and on-skin sensor assembly of the applicator system of FIGS. 5 and 6.

FIG. 19 illustrates a top view of a portion of the needle carrier assembly and hub of FIGS. 5 and 6.

FIGS. 20A and 20B illustrate perspective views of locking features for needles for use in an applicator for an analyte sensor system.

FIGS. 21-23 illustrate several cross-sectional views, and various features and operating positions, of yet another applicator for an on-skin sensor assembly of an analyte sensor system.

FIG. 24 illustrates a perspective view of various features of the applicator system of FIGS. 21-23.

FIG. 25 illustrates a cross-sectional view of a system, according to some examples.

FIGS. 26A-26B illustrate cross-sectional schematic views of an analyte sensor with and without an insertion element.

FIG. 27 illustrates a perspective view of an on-skin sensor assembly disposed upon skin of a host.

FIG. 28 illustrates a perspective view of an overlay patch.

FIG. 29 illustrates a top view of an overlay patch upon a backing layer and covered with a liner.

FIG. 30 illustrates a cross sectional view along line C-C′ in FIG. 29.

FIG. 31 illustrates a perspective view of an on-skin sensor assembly disposed upon skin of a host and with an overlay patch applied to the on-skin sensor assembly.

FIG. 32 illustrates a side cross sectional view of a configuration as shown in FIG. 31.

FIG. 33 illustrates a side cross sectional view of an overlay patch upon a backing layer and covered with a liner.

FIG. 34 illustrates a distal perspective view of an applicator for an overlay patch.

FIG. 35 illustrates a proximal perspective view of the applicator shown in FIG. 34.

FIG. 36 illustrates a side cross sectional view of the applicator shown in FIG. 34.

FIG. 37 illustrates a side cross sectional view of the applicator shown in FIG. 34 orthogonal to the view shown in FIG. 36.

FIG. 38 illustrates a distal perspective view of the applicator shown in FIG. 34 with an overlay patch positioned upon a substrate.

FIG. 39 illustrates a side cross sectional view of an application procedure utilizing the applicator shown in FIG. 34.

FIG. 40 illustrates a side cross sectional view of an application procedure utilizing the applicator shown in FIG. 34.

FIG. 41 illustrates a side cross sectional view of an application procedure utilizing the applicator shown in FIG. 34.

FIG. 42 illustrates a side cross sectional view of an overlay patch and backing layer applied to an on-skin sensor assembly.

FIG. 43 illustrates a side cross sectional view of an overlay patch applied to an on-skin sensor assembly.

FIG. 44 illustrates a perspective view of an applicator for an overlay patch.

FIG. 45 illustrates a top view of the applicator shown in FIG. 44.

FIG. 46 illustrates a perspective view of the applicator shown in FIG. 44 with an overlay patch and a backing layer positioned upon the applicator shown in FIG. 44.

FIG. 47 illustrates a side cross sectional view of the applicator shown in FIG. 44 with an overlay patch and a backing layer positioned upon the applicator.

FIG. 48 illustrates a side cross sectional view of the applicator shown in FIG. 44 with an overlay patch and a backing layer positioned upon the applicator and an alignment body displaced relative to a substrate.

FIG. 49 illustrates a perspective view of an applicator for an overlay patch.

FIG. 50 illustrates a perspective view of an applicator for an overlay patch comprising packaging.

FIG. 51 illustrates a side cross sectional view of the applicator shown in FIG. 50.

FIG. 52 illustrates a side cross sectional view of the applicator shown in FIG. 51 with a central portion deflected.

FIG. 53 illustrates a proximal perspective view of the applicator shown in FIG. 51 with the central portion deflected.

FIG. 54 illustrates a proximal view of an applicator for an overlay patch.

FIG. 55 illustrates a side cross sectional view of the applicator shown in FIG. 54.

FIG. 56 illustrates a proximal view of the applicator shown in FIG. 54 with a central portion deflected.

FIG. 57 illustrates a side cross sectional view of the applicator in the position shown in FIG. 56.

FIG. 58 illustrates a perspective view of an applicator for an overlay patch comprising a cap for an applicator housing.

FIG. 59 illustrates a side cross sectional view of the applicator shown in FIG. 58.

FIG. 60 illustrates a side cross sectional view of the applicator shown in FIG. 58 with an overlay patch applied to a substrate.

FIG. 61 illustrates a side cross sectional view of the applicator shown in FIG. 58 with an alignment body deflected from the position shown in FIG. 60.

FIG. 62 illustrates a perspective view of the alignment body deflected in FIG. 61.

FIG. 63 illustrates a side cross sectional view of an applicator for an overlay patch.

FIG. 64 illustrates a proximal perspective view of an applicator for an overlay patch positioned above an on-skin sensor assembly.

FIG. 65 illustrates an assembly view of the applicator for the overlay patch shown in FIG. 64.

FIG. 66 illustrates a side cross sectional view of the applicator shown in FIG. 65.

FIG. 67 illustrates a side cross sectional view of the applicator shown in FIG. 65 deploying an overlay patch to an on-skin sensor assembly.

FIG. 68 illustrates a side cross sectional view of the applicator shown in FIG. 65 having deployed an overlay patch to an on-skin sensor assembly.

FIG. 69 illustrates a perspective view of an applicator for an overlay patch.

FIG. 70 illustrates a perspective view of the applicator shown in FIG. 69 with an overlay patch and backing layer positioned upon the applicator.

FIG. 71 illustrates a cross sectional view of the applicator shown in FIG. 70 with an overlay patch and backing layer positioned upon the applicator.

FIG. 72 illustrates a cross sectional view of the applicator shown in FIG. 70 applying an overlay patch to an on-skin sensor assembly.

FIG. 73 illustrates a perspective view of an overlay patch having been applied to an on-skin sensor assembly.

FIG. 74 illustrates a perspective assembly view of an applicator and a patch assembly, with a portion of the applicator shown in transparency.

FIG. 75 illustrates a distal perspective view of the applicator shown in FIG. 74.

FIG. 76 illustrates a perspective cross sectional view of the applicator shown in FIG. 74, with a patch assembly coupled thereto.

FIG. 77 illustrates a side cross sectional view of the applicator shown in FIG. 74, with a patch being deployed to an on-skin sensor assembly.

FIG. 78 illustrates a perspective view of the applicator shown in FIG. 74 being withdrawn from an on-skin sensor assembly.

FIG. 79 illustrates a perspective assembly view of an applicator and a patch assembly, with a portion of the applicator shown in transparency.

FIG. 80 illustrates a distal perspective view of the applicator shown in FIG. 79.

FIG. 81 illustrates a perspective cross sectional view of the applicator shown in FIG. 79, with a patch assembly coupled thereto.

FIG. 82 illustrates a side cross sectional view of the applicator shown in FIG. 79, with a patch assembly coupled thereto.

FIG. 83 illustrates a perspective view of the applicator shown in FIG. 79 being positioned upon an on-skin sensor assembly.

FIG. 84 illustrates a side cross sectional view of the applicator shown in FIG. 79, with a patch being deployed to an on-skin sensor assembly.

FIG. 85 illustrates a perspective assembly view of an applicator and a patch assembly, with a portion of the applicator shown in transparency.

FIG. 86 illustrates a side cross sectional view of the applicator shown in FIG. 85, with a patch assembly coupled thereto.

FIG. 87 illustrates a side cross sectional view of the applicator shown in FIG. 85, with a patch being deployed to an on-skin sensor assembly.

FIG. 88 illustrates a perspective view of an applicator.

FIG. 89 illustrates a perspective view of the applicator shown in FIG. 88, with a patch assembly coupled thereto.

FIG. 90 illustrates a side cross sectional view of the applicator shown in FIG. 88, with a patch assembly coupled thereto.

FIG. 91 illustrates a perspective view of the applicator shown in FIG. 88, with a patch assembly being coupled thereto.

FIG. 92 illustrates a distal perspective view of the applicator shown in FIG. 88 being advanced towards an on-skin sensor assembly.

FIG. 93 illustrates a perspective view of the applicator shown in FIG. 88 positioned upon an on-skin sensor assembly with a patch assembly being deployed to the on-skin sensor assembly.

FIG. 94 illustrates a perspective view of an applicator.

FIG. 95 illustrates a side cross sectional view of the applicator shown in FIG. 94.

FIG. 96 illustrates a distal perspective view of the applicator shown in FIG. 94.

FIG. 97 illustrates a perspective view of the applicator shown in FIG. 94 approaching an on-skin sensor assembly.

FIG. 98 illustrates a perspective view of a backing layer being removed from an on-skin sensor assembly shown in FIG. 97.

FIG. 99 illustrates a perspective view of the applicator of FIG. 94 applied to the on-skin sensor assembly shown in FIG. 97.

FIG. 100 illustrates a side cross sectional view of the applicator of FIG. 94 applied to the on-skin sensor assembly shown in FIG. 97.

### DETAILED DESCRIPTION

The following description illustrates some examples of the disclosure in detail. Those of skill in the art will recognize that there are numerous variations and modifications of the disclosure that are encompassed by its scope. Accordingly, the description of a certain example should not be deemed to limit the scope of the present disclosure.

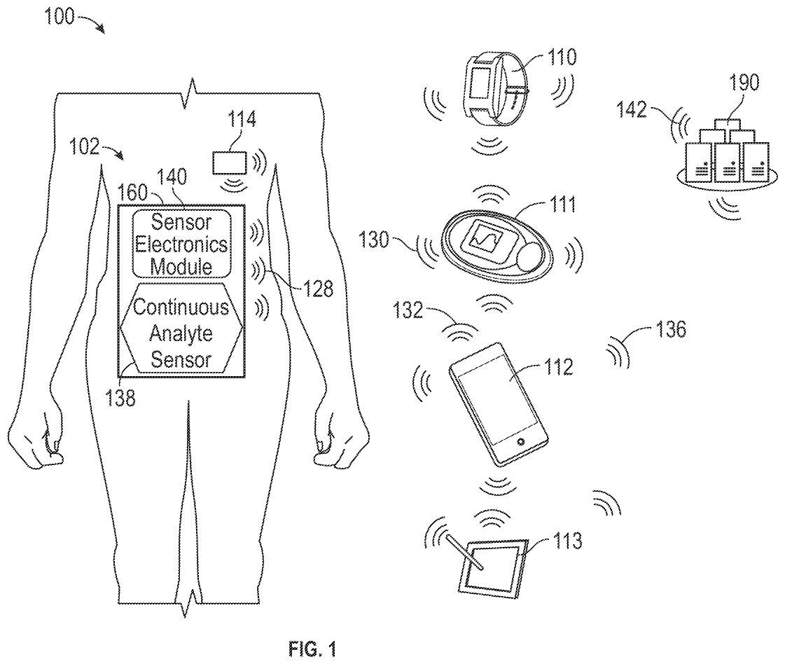

FIG. 1 is a diagram depicting an example medical device system according to examples herein. The medical device system in examples may comprise a continuous analyte monitoring system 100. The continuous analyte monitoring system 100 may include an analyte sensor system 102 comprising an on-skin sensor assembly 160 configured to be fastened to the skin of a host via a base (not shown).

In examples, other forms of medical device systems may be utilized, including other forms of monitoring systems, medicament delivery systems, or other therapeutic systems. In examples, an on-skin wearable medical device may be utilized that may comprise an on-skin sensor assembly, or a medicament delivery medical device, among other forms of on-skin wearable medical devices.

As shown in FIG. 1, the analyte sensor system 102 may be operatively connected to a host and a plurality of display devices 110-114 according to certain aspects of the present disclosure. Example display devices 110-114 may include computers such as smartphones, smartwatches, tablet computers, laptop computers, and desktop computers. In some examples, display devices 110-114 may be Apple Watches, iPhones, and iPads made by Apple Inc., or iOS, Windows, or Android operating system devices. It should be noted that display device 114 alternatively or in addition to being a display device, may be a medicament delivery device that can act cooperatively with analyte sensor system 102 to deliver medicaments to the host. Analyte sensor system 102 may include a sensor electronics module 140 and a continuous analyte sensor 138 associated with sensor electronics module 140. Sensor electronics module 140 may be in direct wireless communication with one or more of the plurality of display devices 110-114 via wireless communications signals. As will be discussed in greater detail below, display devices 110-114 may also communicate amongst each other and/or through each other to analyte sensor system 102. For case of reference, wireless communications signals from analyte sensor system 102 to display devices 110-114 can be referred to as “uplink” signals 128. Wireless communications signals from, e.g., display devices 110-114 to analyte sensor system 102 can be referred to as “downlink” signals 130. Wireless communication signals between two or more of display devices 110-114 may be referred to as “crosslink” signals 132. Additionally, wireless communication signals can include data transmitted by one or more of display devices 110-113 via “long-range” uplink signals 136 (e.g., cellular signals) to one or more remote servers 190 or network entities, such as cloud-based servers or databases, and receive long-range downlink signals 142 transmitted by remote servers 190.

In examples shown by FIG. 1, one of the plurality of display devices may be a custom display device 111 specially designed for displaying certain types of displayable sensor information associated with analyte values received from the sensor electronics module 140 (e.g., a numerical value and an arrow, in some examples). In some examples, one of the plurality of display devices may be a handheld device 112, such as a mobile phone based on the Android, IOS operating systems or other operating system, a palm-top computer and the like, where handheld device 112 may have a relatively larger display and be configured to display a graphical representation of the continuous sensor data (e.g., including current and historic data). Other display devices can include other hand-held devices, such as a tablet 113, a smart watch 110, a medicament delivery device 114, a blood glucose meter, and/or a desktop or laptop computer.

It should be understood that in the case of display device 114, which may be a medicament delivery device in addition to or instead of a display device, the alerts and/or sensor information provided by continuous analyte sensor 138 vis-à-vis sensor electronics module 140, can be used to initiate and/or regulate the delivery of the medicament to host.

During use, a sensing portion of sensor 138 may be disposed under the host's skin and a contact portion of sensor 138 can be electrically connected to sensor electronics module 140. Electronics module 140 can be engaged with a housing (e.g., a base) which is attached to a patch that may engage the skin of the host. The housing may comprise a wearable housing. The patch may be an adhesive patch in examples. In some examples, electronics module 140 is integrally formed with the housing. Furthermore, electronics module 140 may be disposable and directly coupled to the patch.

Continuous analyte sensor system 100 can include a sensor configuration that provides an output signal indicative of a concentration of an analyte. The output signal including (e.g., sensor data, such as a raw data stream, filtered data, smoothed data, and/or otherwise transformed sensor data) is sent to the receiver.

In some examples, analyte sensor system 102 includes a transcutaneous glucose sensor, such as is described in U.S. Patent Publication No. 2011/0027127, the entire contents of which are hereby incorporated by reference. In some examples, sensor system 102 includes a continuous glucose sensor and comprises a transcutaneous sensor (e.g., as described in U.S. Pat. No. 6,565,509, as described in U.S. Pat. No. 6,579,690, and/or as described in U.S. Pat. No. 6,484,046). The contents of U.S. Pat. Nos. 6,565,509, 6,579,690, and 6,484,046 are hereby incorporated by reference in their entirety.

Various signal processing techniques and glucose monitoring system examples suitable for use with the examples described herein are described in U.S. Patent Publication No. 2005/0203360 and U.S. Patent Publication No. 2009/0192745, the contents of which are hereby incorporated by reference in their entirety. The sensor can extend through a housing, which can maintain sensor 138 on, in or under the skin and/or can provide for electrical connection of sensor 138 to sensor electronics in sensor electronics module 140.

In some examples, description of a base, a housing, a wearable, and/or a transmitter of on-skin sensor assembly 160 may be interchangeable. In other examples, a base and a housing of on-skin sensor assembly 160 may be different in the sense that they may be separate components from sensor electronics module 140, e.g., from a transmitter or receiver.

In several examples, sensor 138 is in a form of a wire. A distal end of the wire can be formed, e.g., having a conical shape (to facilitate inserting the wire into the tissue of the host). Sensor 138 may comprise an elongate analyte sensor, and may include an elongate conductive body, such as an elongate conductive core (e.g., a metal wire) or an elongate conductive core coated with one, two, three, four, five, or more layers of material, each of which may or may not be conductive. The elongate analyte sensor may be long and thin, yet flexible and strong. For example, in some examples, the smallest dimension of the elongate conductive body is less than 0.1 inches, less than 0.075 inches, less than 0.05 inches, less than 0.025 inches, less than 0.01 inches, less than 0.004 inches, less than 0.002 inches, less than 0.001 inches, and/or less than 0.0005 inches.

Sensor 138 may have a circular shaped cross section. In some examples, the cross section of the elongated conductive body can be ovoid, rectangular, triangular, polyhedral, star-shaped, C-shaped, T-shaped, X-shaped, Y-shaped, irregular, or the like. In some examples, a conductive wire electrode is employed as a core. In other examples, sensor 138 may be disposed on a substantially planar substrate. To such an electrode, one or two additional conducting layers may be added (e.g., with intervening insulating layers provided for electrical isolation). The conductive layers can be comprised of any suitable material. In certain examples, it may be desirable to employ a conductive layer comprising conductive particles (i.e., particles of a conductive material) in a polymer or other binder.

In some examples, the materials used to form the elongate conductive body (e.g., stainless steel, titanium, tantalum, platinum, platinum-iridium, iridium, certain polymers, and/or the like) can be strong and hard, and therefore can be resistant to breakage. For example, in several examples, the ultimate tensile strength of the elongated conductive body is greater than 80 kPsi and less than 140 kPsi, and/or the Young's modulus of the elongate conductive body is greater than 160 GPa and less than 220 GPa. The yield strength of the elongate conductive body can be greater than 58 kPsi and less than 2200 kPsi.

Electronics module 140 can be releasably or permanently coupled to sensor 138. Electronics module 140 can include electronic circuitry associated with measuring and processing the continuous analyte sensor data. Electronics module 140 can be configured to perform algorithms associated with processing and calibration of the sensor data. For example, electronics module 140 can provide various aspects of the functionality of a sensor electronics module as described in U.S. Patent Publication No. 2009/0240120 and U.S. Patent Publication No. 2012/0078071, the entire contents of which are incorporated by reference herein. Electronics module 140 may include hardware, firmware, and/or software that enable measurement of levels of the analyte via a glucose sensor, such as sensor 138.

For example, electronics module 140 can include a potentiostat, a power source for providing power to sensor 138, signal processing components, data storage components, and a communication module (e.g., a telemetry module) for one-way or two-way data communication between electronics module 140 and one or more receivers, repeaters, and/or display devices, such as devices 110-114. Electronic components can be affixed to a printed circuit board (PCB), or the like, and can take a variety of forms. The electronic components can take the form of an integrated circuit (IC), such as an Application-Specific Integrated Circuit (ASIC), a microcontroller, and/or a processor. The electronics module 140 may include sensor electronics that are configured to process sensor information, such as storing data, analyzing data streams, calibrating analyte sensor data, estimating analyte values, comparing estimated analyte values with time-corresponding measured analyte values, analyzing a variation of estimated analyte values, and the like. Examples of systems and methods for processing sensor analyte data are described in more detail in U.S. Pat. Nos. 7,310,544, 6,931,327, U.S. Patent Publication No. 2005/0043598, U.S. Patent Publication No. 2007/0032706, U.S. Patent Publication No. 2007/0016381, U.S. Patent Publication No. 2008/0033254, U.S. Patent Publication No. 2005/0203360, U.S. Patent Publication No. 2005/0154271, U.S. Patent Publication No. 2005/0192557, U.S. Patent Publication No. 2006/0222566, U.S. Patent Publication No. 2007/0203966 and U.S. Patent Publication No. 2007/0208245, the contents of which are hereby incorporated by reference in their entirety. Electronics module 140 may communicate with the devices 110-114, and/or any number of additional devices, via any suitable communication protocol. Example communication methods or protocols include radio frequency; Bluetooth; universal serial bus; any of the wireless local area network (WLAN) communication standards, including the IEEE 802.11, 802.15, 802.20, 802.22 and other 802 communication protocols; ZigBee; wireless (e.g., cellular) telecommunication; paging network communication; magnetic induction; satellite data communication; a proprietary communication protocol, open source communication protocol, and/or any suitable wireless communication method.

Additional sensor information is described in U.S. Pat. Nos. 7,497,827 and 8,828,201. The entire contents of U.S. Pat. Nos. 7,497,827 and 8,828,201 are incorporated by reference herein.

Any sensor shown or described herein can be an analyte sensor; a glucose sensor; and/or any other suitable sensor. A sensor described in the context of any example can be any sensor described herein or incorporated by reference. Sensors shown or described herein can be configured to sense, measure, detect, and/or interact with any analyte.

As used herein, the term “analyte” is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, cerebral spinal fluid, lymph fluid, urine, sweat, saliva, etc.) that can be analyzed. Analytes can include naturally occurring substances, artificial substances, metabolites, or reaction products.

In some examples, the analyte for measurement by the sensing regions, devices, systems, and methods is glucose. However, other analytes are contemplated as well, including, but not limited to ketone bodies; acetyl-CoA; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; cortisol; testosterone; choline; creatine kinase; creatine kinase MM isoenzyme; cyclosporin A; d-penicillamine; de-cthylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; triglycerides; glycerol; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); acetone (e.g., succinylacetone); acetoacetic acid; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid or endogenous, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and intermediaries in the Citric Acid Cycle.

Any of the features described in the context of at least FIG. 1 can be applicable to all aspects and examples identified herein. Moreover, any of the features of an example is independently combinable, partly or wholly with other examples described herein in any way, e.g., one, two, or three or more examples may be combinable in whole or in part. Further, any of the features of an example may be made optional to other aspects or examples. Any aspect or example of a method can be performed by a system or apparatus of another aspect or example, and any aspect or example of a system can be configured to perform a method of another aspect or example.

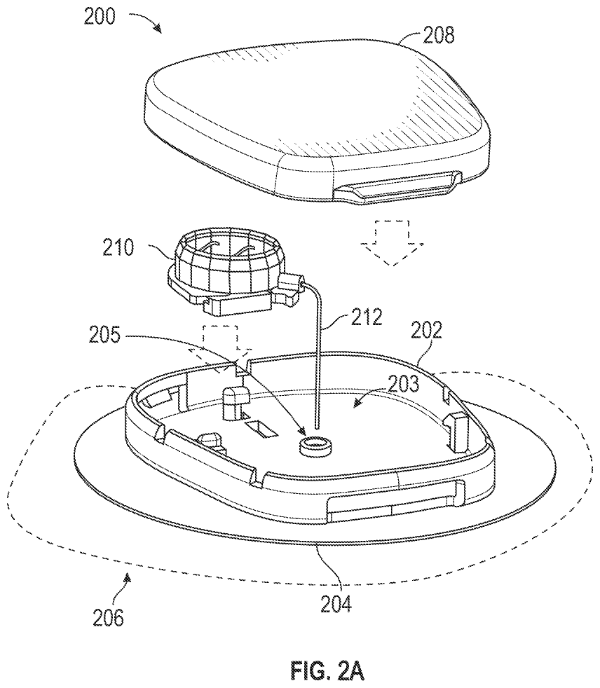

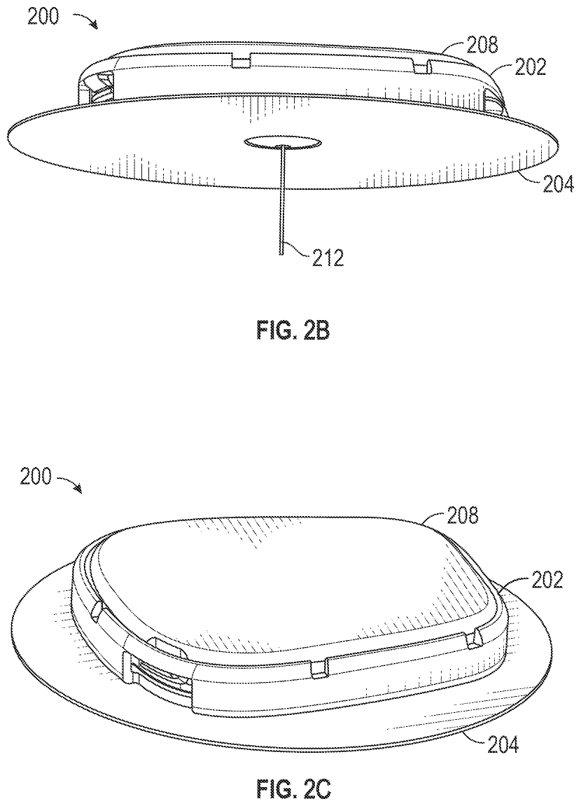

FIG. 2A illustrates a perspective view of an exemplary on-skin wearable medical device, in the form of an on-skin sensor assembly 200, which is configured to be deployed to skin. The on-skin sensor assembly 200 may include a housing or base 202. The housing or base 202 may be configured to be worn on skin of a host and may include a distal surface for facing towards the skin and a proximal surface 203 facing opposite the distal surface. The housing or base 202 may include an opening 205 for an insertion element to be retracted proximally through from the skin. A patch 204 such as an adhesive patch can couple the base 202 to the skin 206 of the host. The patch 204 may be positioned on the distal surface of the housing or base 202. In some examples, the adhesive patch 204 may include an engaging surface for engaging the skin and including an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. In examples, the patch 204 may be an underlay patch. The underlay patch may be configured to be positioned beneath an overlay patch. An on-skin sensor assembly 200 may comprise an electronics unit 208 (e.g., a transmitter) which may further comprise a glucose sensor module 210 coupled to an analyte sensor such as a transcutaneous analyte sensor (e.g., a glucose sensor) 212 and to base 202.

The applicator system can engage the adhesive patch 204 or underlay patch to skin 206. The glucose sensor module 210 may be secured to base 202 (e.g., via retention elements such as snap fits and/or interference features, adhesive, welding, etc.) to ensure analyte sensor 212 (e.g., glucose sensor) is coupled to base 202. In alternative examples, the sensor module 210 and base 202 are preassembled or manufactured as a single component.

After on-skin sensor assembly 200 is deployed to a host's or user's skin, a user (or an applicator) can couple electronics unit 208 (e.g., a transmitter) to on-skin sensor assembly 200 via retention elements such as snap fits and/or interference features. Electronics unit 208 can measure and/or analyze glucose indicators sensed by transcutaneous analyte sensor (e.g., a glucose sensor) 212. Electronics unit 208 can transmit information (e.g., measurements, analyte data, glucose data) to a remotely located device (e.g., 110-114 shown in FIG. 1).

On-skin sensor assembly 200 may be attached to the host with use of an applicator adapted to provide convenient and secure application. Such an applicator may also be used for attaching electronics unit 208 to base 202, inserting sensor 212 through the host's skin, and/or connecting sensor 212 to electronics unit 208. Once electronics unit 208 is engaged with the base and sensor 212 has been inserted into the skin (and is connected to the electronics unit 208), the sensor assembly can detach from the applicator.

FIG. 2B illustrates a perspective view of electronics unit 208 coupled to base 202 via retention elements such as snap fits and/or interference features. In some examples, electronics unit 208 and base 202 are coupled by adhesive, welding, or other bonding techniques. Patch 204, on a distal surface of base 202, is configured to couple sensor assembly 200 to the skin.

FIG. 2C illustrates a perspective view of on-skin sensor assembly 200. On-skin sensor assembly 200 may be disposable or reusable. FIG. 2C further illustrates electronics unit 208 coupled to a base 202, and adhesive patch 204 or underlay patch configured to be attached to on-skin sensor assembly 200, which, when combined, may be held within the applicator.

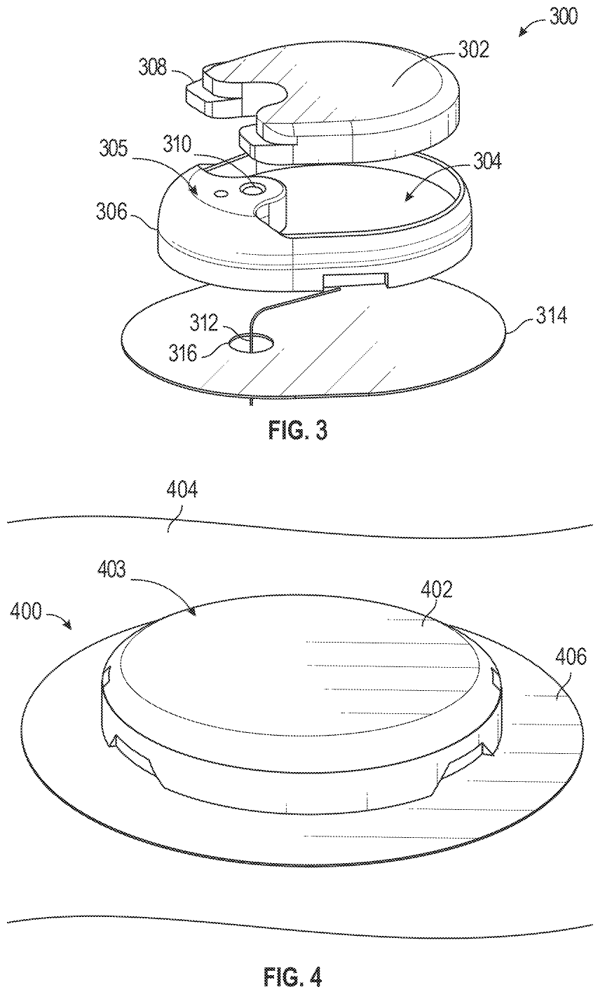

FIG. 3 illustrates an example of an on-skin wearable medical device in the form of an on-skin sensor assembly 300 with an electronics unit 302 configured to insert into a cavity 304 of the base or housing 306. The base or housing 306 may be configured to be worn on skin of a host and may include a distal surface for facing towards the skin and a proximal surface 305 facing opposite the distal surface. The electronics unit 302 may include one or more tabs 308 that couple to a portion of the housing 306 and allow the electronics unit 302 to be retained by the housing 306. The housing 306 may include an opening 310 for an insertion element to be retracted proximally through from the skin. The opening 310 may allow the insertion element (such as a needle) to pass through to deploy the transcutaneous analyte sensor 312 to the skin. The patch 314 may further include an aperture 316 that may allow the sensor 312 and the insertion element to pass through. The electronics unit 302 may couple to the housing 306 prior to or following deployment of the sensor 312 to the host's skin.

FIG. 4 illustrate an example of an on-skin wearable medical device in the form of an on-skin sensor assembly 400, in which the electronics unit is integral with the housing 402. The housing 402 may be configured to be worn on skin of a host and may include a distal surface for facing towards the skin and a proximal surface 403 facing opposite the distal surface. The on-skin sensor assembly 400 is shown on the skin 404, with the patch 406 engaging the skin 404.

The examples of FIGS. 2A-4 may each include an engaging surface for engaging the skin. The engaging surface may be positioned on the patch in examples, for example on a distal surface of the patch or may have another position in examples. The engaging surface may comprise an adhesive surface in examples configured to adhere to the skin. The adhesive can be configured for adhering to skin. Additional adhesive information is described in U.S. Pat. No. 11,219,413, which was filed on Aug. 25, 2015. The entire contents of U.S. Pat. No. 11,219,413 are incorporated by reference herein. The engaging surface in examples may be covered with a liner prior to deployment to the host's skin.

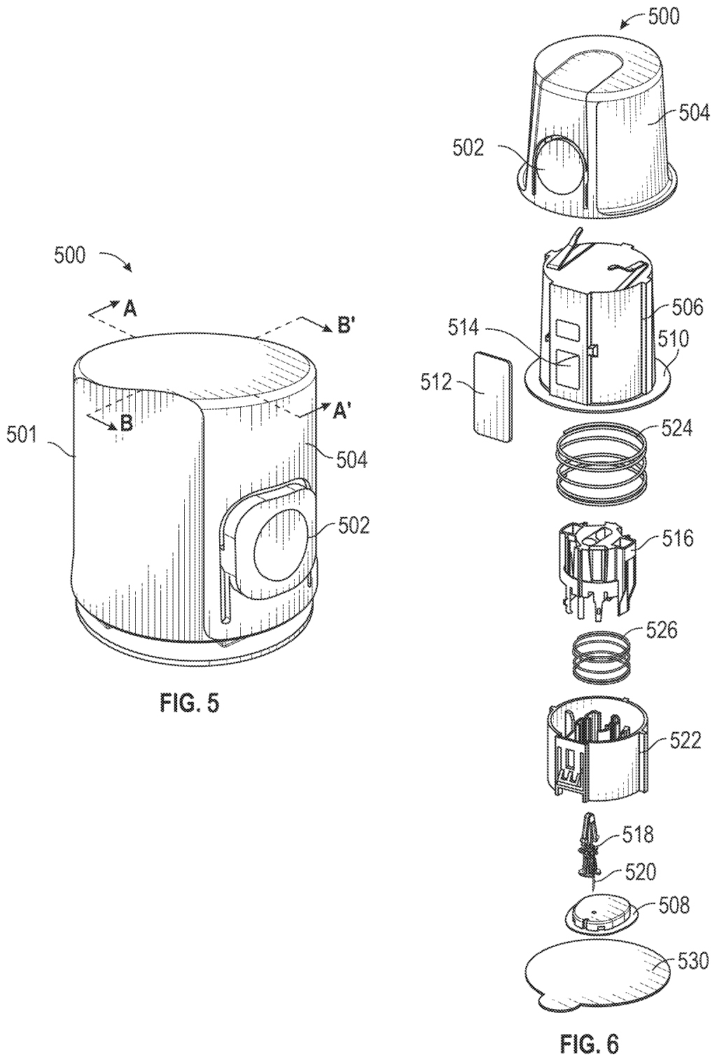

FIG. 5 illustrates a system for deploying an on-skin wearable medical device to skin. The system may comprise an applicator system in examples. The system may include an applicator for an on-skin sensor assembly of an analyte sensor system, according to some examples. In examples, other forms of systems may be utilized.

The applicator 500 may include an applicator housing 501, which may include an outer housing 504 and an inner housing 506, and other forms of housings in examples. The applicator housing 501 may be configured to retain the on-skin wearable medical device in examples. The applicator 500 may include a deployment mechanism that may be configured to deploy the on-skin wearable medical device to skin. The deployment mechanism, for example, may include an engagement portion for retaining the on-skin wearable medical device and releasing the on-skin wearable medical device from the applicator housing 501 to the skin in examples. The engagement portion may include one or more retention element(s). The deployment mechanism may include an insertion assembly for inserting at least a portion of the on-skin wearable medical device into the skin. The insertion assembly may drive a portion of the on-skin wearable medical device, such as the insertion element and the sensor, into the skin of the host. The deployment mechanism may include a retraction assembly for retracting the portion of the on-skin wearable medical device from the skin, such as an insertion element.

In examples, the applicator 500 may include an activation element 502 disposed on a side of applicator 500, for example, on a side of an outer housing 504 of applicator 500. In some examples, activation element 502 may be a button, a switch, a toggle, a slide, a trigger, a knob, a rotating member, a portion of applicator 500 that deforms and/or flexes or any other suitable mechanism for activating an insertion and/or retraction assembly of applicator 500. In some examples, activation element 502 may be disposed in any location, e.g., a top, upper side, lower side, or any other location of applicator 500. Applicator 500 may be large enough for a host to grasp with a hand and push, or otherwise activate, activation element 502 with, for example, a thumb, or with an index finger and/or a middle finger.

Applicator 500 may be configured with one or more safety features such that applicator 500 is prevented from activating until the safety feature is deactivated. In one example, the one or more safety features prevents applicator 500 from activating unless applicator 500 is pressed against the skin of a host with sufficient force. Moreover, as will be described in more detail in connection with one or more of FIGS. 6-20B below, applicator 500 may be further configured such that one or more components therein retract based at least in part on the one or more components pushing against the skin of the host with a force exceeding a predetermined threshold, rather than based on the one or more components translating beyond a predetermined and static distal position. In other words, applicator 500 may implement force-based retraction triggering rather than being limited to displacement-based retraction triggering.

FIG. 6 illustrates an exploded perspective view of applicator 500 of FIG. 5, according to some examples. Applicator 500 may include outer applicator housing 504 comprising activation element 502. The outer applicator housing 504 may be configured to be gripped by a user in examples. Outer applicator housing 504 may be configured to translate in a distal direction by a force applied by a host to applicator 500, specifically to inner housing 506, thereby aligning activation element 502 in a position that allows applicator 500 to fire. Further explanation of the alignment process will be explained below.

Applicator 500 further comprises inner housing 506, configured to house at least one or more mechanisms utilized to apply on-skin sensor assembly 508 to skin of a host. A distal surface 510 of a bottom opening of inner housing 506 may define a bottom surface of applicator 500. In some examples, upon pressing applicator 500 against skin of the host, skin may deform in a substantially convex shape at distal surface 510 such that at least a portion of a surface of skin disposed at the bottom opening of applicator inner housing 506 extends into the bottom opening of inner housing 506 beyond a plane defined by distal surface 510 in a proximal direction.

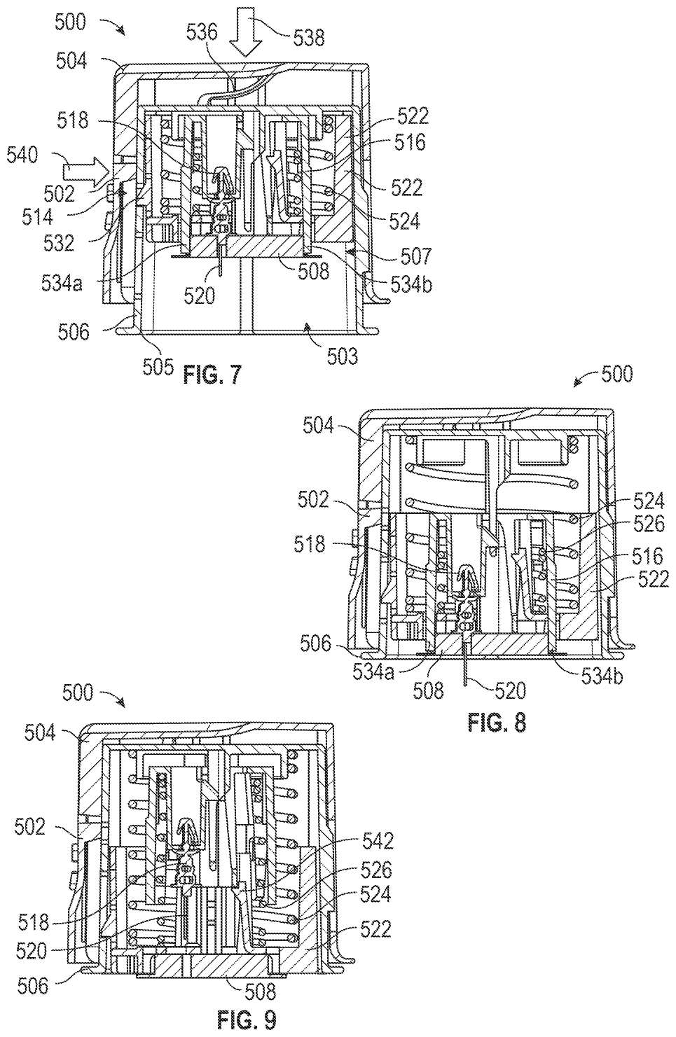

As shown in FIG. 7, the housing 501, and particularly the inner housing 506 may include an internal cavity 503 for retaining the on-skin wearable medical device. The internal cavity 503 may have a distal end portion 505 at the opening for on-skin wearable medical device to be deployed from. A proximal end portion 507 of the internal cavity 503 may include the on-skin wearable medical device coupled to the needle carrier assembly 516.

Referring back to FIG. 6, in some examples, a first barrier layer 512 may be disposed over one or more apertures in inner housing 506, for example, an aperture 514 through which at least a portion of activation element 502 may be configured to extend through during activation of applicator 500. In such examples, a portion of activation element 502 may be configured to pierce or deform first barrier layer 512 upon activation of applicator 500. First barrier layer 512 may comprise a gas permeable material such as Tyvek, or a non-gas permeable material such as metallic foil, polymer film, elastomer, or any other suitable material.

Applicator 500 may further comprise a needle carrier assembly 516, including a needle hub 518 configured to couple an insertion element 520 to needle carrier assembly 516. In some other examples, insertion element 520 may be directly coupled to needle carrier assembly 516. Insertion element 520 is configured to insert sensor of on-skin sensor assembly 508 into skin of the host. In some examples, the insertion element comprises a needle, for example, an open sided-needle, a needle with a deflected-tip, a curved needle, a polymer-coated needle, a hypodermic needle, or any other suitable type of needle or structure. In yet other examples, insertion element 520 may be integrally formed with sensor and may be sufficiently rigid to be inserted partially into skin of the host with minimal or no structural support.

Applicator 500 may further include a holder 522 releasably coupled to needle carrier assembly 516 and configured to guide needle carrier assembly 516 and on-skin sensor assembly 508 while coupled to needle carrier assembly 516, e.g., at least during translation from a proximal position to a distal insertion position. As will be described in more detail below, on-skin sensor assembly 508 may be stripped or released from holder 522 and/or needle carrier assembly 516 once on-skin sensor assembly 508 is disposed on skin of the host. For example, an engagement portion or one or more retention elements may release the on-skin wearable medical device from the applicator housing 501.

Applicator 500 may further comprise an insertion assembly configured to translate insertion element 520, needle hub 518, needle carrier assembly 516, and on-skin sensor assembly 508 from a proximal position, in the distal direction, to a distal insertion position. Such an insertion assembly may include at least one spring for inserting at least a portion of the on-skin wearable device into the skin. The insertion assembly may include a first spring 524. First spring 524 may be a compression spring, or any suitable type of spring, and may have a first end in contact with or coupled to inner applicator housing 506 and a second end in contact with or coupled to holder 522. First spring 524 is configured to, upon activation of the insertion assembly, translate holder 522, needle carrier assembly 516, needle hub 518, insertion element 520 and on-skin sensor assembly 508, in the distal direction to the distal insertion position. Substantially at the distal insertion position, needle carrier assembly 516 may decouple from holder 522 and on-skin sensor assembly 508.

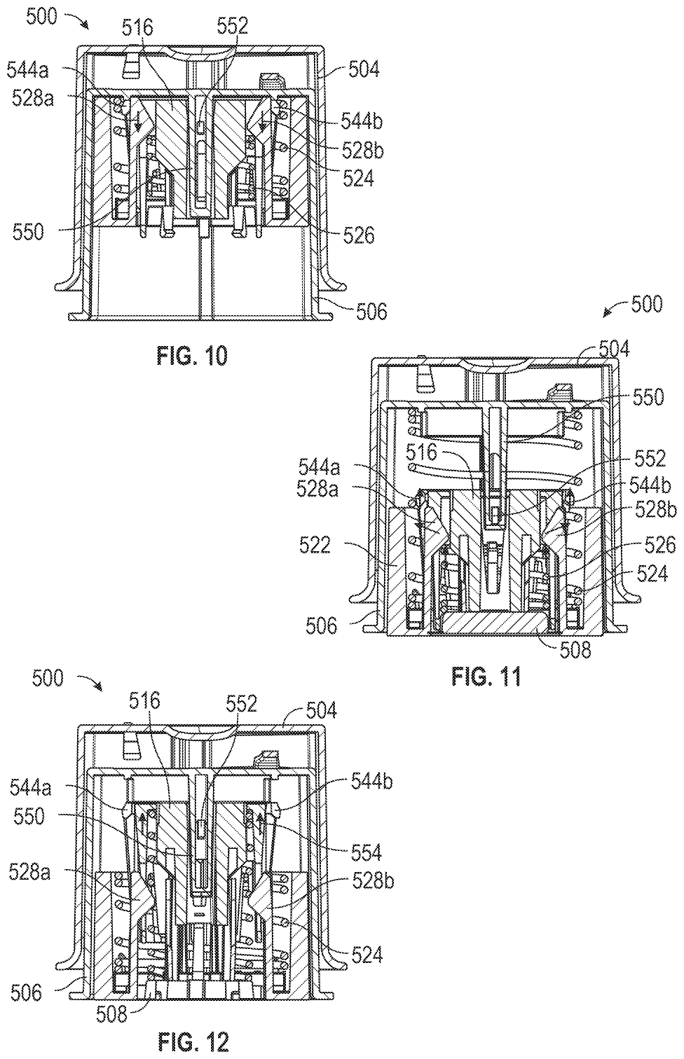

Applicator 500 may further comprise a retraction assembly for retracting the insertion element (e.g., needle) from the skin. The retraction assembly may be configured to translate needle carrier assembly 516, needle hub 518 and insertion element 520, in the proximal direction, from the distal insertion position to a proximal retracted position. In some examples the initial proximal position may be the same as the proximal retracted position. In other examples, the initial proximal position may be different from the proximal retracted position. Such a retraction assembly may include at least one spring. The retraction assembly may include a second spring 526. Second spring 526 may be a compression spring, or any suitable type of spring, and may have a first end contacting or coupled to holder 522 and a second end in contact with or coupled to at least one spring retention element (e.g., 528a, 528b in FIGS. 10-14), at least until retraction. Second spring 526 is configured to translate needle carrier assembly 516, needle hub 518, and insertion element 520 in the proximal direction from the distal insertion position to the proximal retracted position in response to on-skin sensor assembly 508 contacting skin of the host, and/or reaching a limit of travel with a force exceeding a predetermined threshold sufficient to cause first end of second spring 526 to overcome the at least one spring retention element (e.g., 528a, 528b in FIGS. 10-14). In some examples, a stop feature (not shown) may be disposed at a bottom of applicator 500, e.g., on a distal portion of inner housing 506. Such a stop feature may be configured to contact one or more of on-skin sensor assembly 508, needle carrier assembly 516, or holder 522 in the distal insertion position.

In some examples, a second barrier layer 530 may be disposed over the bottom opening of inner housing 506. Second barrier layer 530 may comprise a gas permeable material such as Tyvek, or a non-gas permeable material such as metallic foil, film. In some examples, second barrier layer 530 may be removed by the host prior to use of applicator 500. In examples comprising one or both of first and second barrier layers 512, 530, such layers may provide a sterile environment between applicator 500 and the outside environment and/or may allow ingress and egress of gas such as during sterilization.

A brief description of some aspects of the operation of applicator 500 follows with respect to FIGS. 7-9, which illustrate several cross-sectional views of applicator 500 of FIGS. 5 and 6 during operation, according to some examples. FIGS. 7-9 may correspond to applicator 500 cut along the section line A-A′ shown in FIG. 5, for example.

FIG. 7 illustrates a state of applicator 500 prior to activation. Holder 522 comprises an insertion assembly retention element 532 configured to contact inner housing 506, thereby immobilizing holder 522, needle carrier assembly 516, needle hub 518, insertion element 520 and on-skin sensor assembly 508, in the pre-activated state.

Needle carrier assembly 516 includes an engagement portion of the applicator. The engagement portion may comprise a plurality of wearable retention and/or alignment elements 534a, 534b configured to extend through holder 522 and releasably couple on-skin sensor assembly 508 to holder 522 and/or to needle carrier assembly 516. The engagement portion may have other configurations in examples. Wearable retention elements 534a, 534b may comprise, e.g., arms, deflection element, tabs, detents, snaps or any other features capable of a retaining function. In some examples, wearable retention elements 534a, 534b may extend around rather than through holder 522. Although two wearable retention elements are illustrated, any number of wearable retention elements are contemplated. In some examples, wearable retention element(s) 534a, 534b may comprise snap fits, friction fits, interference features, elastomeric grips and/or adhesives configured to couple on-skin sensor assembly 508 with needle carrier assembly 516 and/or holder 522.

Inner housing 506 may comprise a spring 536 configured to contact outer housing 504 and maintain a predetermined spacing between outer housing 504 and inner housing 506 in the pre-activation orientation of FIG. 7. Spring 536 may be a compression spring, leaf spring, flex arm spring, a piece of foam or rubber, etc. In some other examples, outer housing 504 may comprise spring 536 and spring 536 may be configured to contact inner housing 506, in a reverse fashion from that shown in FIG. 7.

Activation of applicator 500 may include a host pressing applicator 500 against their skin with sufficient force to translate outer housing 504 in a distal direction, as shown by arrow 538, toward and with respect to inner housing 506 until activation element 502 is aligned with aperture 514 of inner housing 506 and insertion assembly retention element 532 of holder 522. Insertion assembly retention element 532 may comprise, e.g., an arm, a deflection element, a tab, a detent, a snap or any other feature capable of a retaining function. Once such an alignment is achieved, a host may initiate (e.g. pushing) activation element 502, as shown by arrow 540, thereby deflecting insertion assembly retention element 532 sufficiently to release holder 522 from inner housing 506. In some other examples, applicator 500 may be configured such that activation element 502 may be activated first, but that actual insertion is not triggered until outer housing 504 is translated sufficiently in the distal direction toward and with respect to inner housing 506. In yet other examples, activation element 502 may be biased toward a center of applicator 500 such that activation element 502 need not be explicitly activated by the host but, instead, activation element 502 may be configured to automatically initiate insertion upon outer housing 504 being translated sufficiently in the distal direction toward and with respect to inner housing 506.

Such configurations provide several benefits. First, translation of outer housing 504 with respect to inner housing 506 before activation provides a measure of drop protection such that if applicator 500 is accidentally dropped, it may not prematurely fire. Second, spring 536 provides a force bias that the host has to affirmatively overcome by pressing applicator 500 into their skin prior to firing, thereby reducing the probability of activating applicator 500 before it is properly positioned. Further, the host may decide to not fire applicator 500 and discontinue pressing applicator 500 against their skin, in which spring 536 will bias against outer housing 504 and allow outer housing 504 to return to its initial state.

Holder 522, needle carrier assembly 516, needle hub 518, insertion element 520, on-skin sensor assembly 508, first spring 524 and second spring 526 are all shown in pre-activation positions in FIG. 7.

FIG. 8 illustrates applicator 500 during insertion of on-skin sensor assembly 508 but before retraction of needle carrier assembly 516. First spring 524 drives holder 522, needle carrier assembly 516, needle hub 518, insertion element 520, and on-skin sensor assembly 508, in the distal direction toward the distal insertion position. FIG. 8 illustrates a position where on-skin sensor assembly 508 is in contact with skin of the host but where holder 522 is not yet fully driven, by first spring 524, into contact with on-skin sensor assembly 508 or skin of the host.

In some examples, masses of each of holder 522, needle carrier assembly 516, needle hub 518, insertion element 520, and on-skin sensor assembly 508 may be specifically designed to reduce or substantially eliminate a tendency of needle carrier assembly 516, needle hub 518, insertion element 520, and on-skin sensor assembly 508 to detach due to inertial forces from holder 522 while being driven in the distal direction during insertion. In some examples, a force exerted by first spring 524 may be selected to be sufficient for proper operation of applicator 500, while not so large as to further exacerbate such above-described inertially triggered detachment. In some examples, a spring (not shown) may be configured to exert a force against a portion of needle carrier assembly 516, for example in a distal direction, sufficient to prevent needle carrier assembly 516 from inertially triggered detaching from holder 522 during insertion.

FIG. 9 illustrates applicator 500 during activation, as needle carrier assembly 516, needle hub 518 and insertion element 520 are retracted in the proximal direction by second spring 526. In FIG. 9, first spring 524 has fully driven on-skin sensor assembly 508 to the skin of the host. In this position, second spring 526 is released from spring retention elements (e.g., 528a, 528b in FIGS. 10-14) and drives needle carrier assembly 516, needle hub 518, and insertion element 520 in the proximal direction from the distal insertion position. Upon needle carrier assembly 516 reaching the proximal retraction position, needle carrier retention element 542 of holder 522 engages with needle carrier assembly 516, thereby maintaining needle carrier assembly 516, needle hub 518 and insertion element 520 in a locked, retracted position limiting access to insertion element 520. Needle carrier retention element 542 may comprise, e.g., an arm, a deflection element, a tab, a detent, a snap or any other feature capable of a retaining function. In this retracted position, needle carrier assembly 516, needle hub 518, and insertion element 520 is prevented from travelling in a distal direction.

A further description of some aspects of the operation of applicator 500 follows with respect to FIGS. 10-12, which illustrate several cross-sectional views of applicator 500 of FIGS. 5 and 6 during operation, according to some examples. FIGS. 10-12 may correspond to applicator 500 cut along the section line B-B′ shown in FIG. 5, for example. For case of illustration, needle hub 518 and insertion element 520 are not shown in FIGS. 10-12.

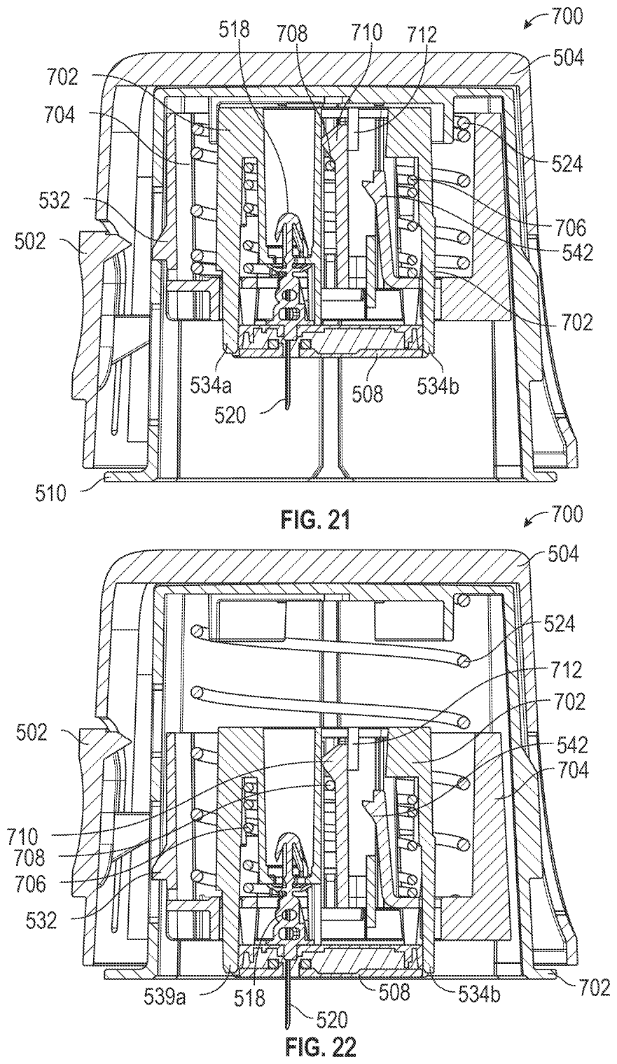

FIG. 10 illustrates a state of applicator 500 prior to activation. For case of illustration, on-skin sensor assembly 508 is not illustrated in FIG. 10. Holder 522 comprises spring retention elements 528a, 528b configured to contact and retain a first end of second spring 526 in the pre-activated state, e.g., during insertion, while a second end of spring 526 is in contact with needle carrier assembly 516. Spring retention elements 528a, 528b may comprise, e.g., arms, deflection element, tabs, detents, snaps or any other features capable of a retaining function. Although two spring retention elements 528a, 528b are shown, at least one spring retention element is contemplated. In some examples, applicator 500 may include one spring retention element, as shown in FIGS. 21-24. In some examples, applicator 500 may include three spring retention elements. In some examples, applicator 500 may include four spring retention elements. In some examples, spring retention elements 528a, 528b are deflectable arms, rigid arms, deformable features, snaps, catches, or hooks. In some examples, spring retention elements 528a, 528b may be actively deflected by one or more features within applicator 500.

Needle carrier assembly 516 comprises backstop features 544a, 544b, configured to prevent lateral deflection of spring retention elements 528a, 528b in the proximal starting position, e.g., at least during insertion, thereby supporting retention of second spring 526 between spring retention elements 528a, 528b and holder 522 until retraction. Although two backstop features are illustrated, any number of backstop features are contemplated. The number of backstop features may equal the number of spring retention elements.

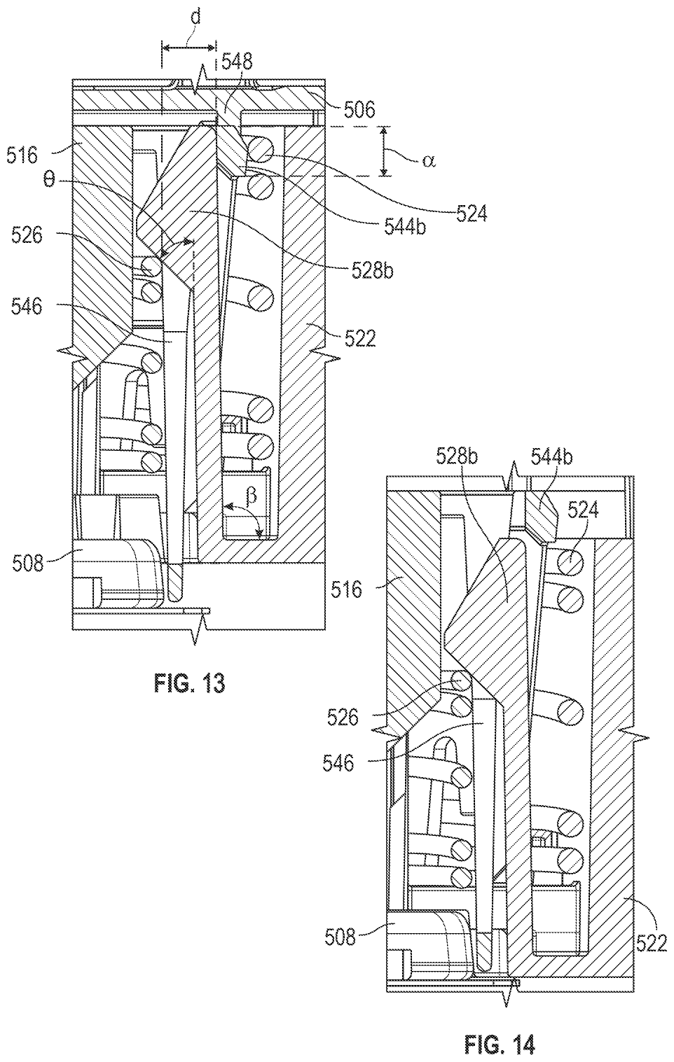

FIG. 13 illustrates a magnified view of spring retention element 528b and backstop feature 544b. In FIG. 13, first spring 524 is driving holder 522, needle carrier assembly 516 and on-skin sensor assembly 508, in the distal direction toward the distal inserted position. Backstop feature 544b is shown engaged to spring retention element 528b, preventing spring retention element 528b from deflecting laterally, thereby preventing second spring 526 from releasing. As shown in FIG. 13, a proximal end of spring retention element 528b may be offset from a distal end of backstop feature 544b by a distance a. In some examples, distance a is the length required for spring retention element 528b to traverse along backstop feature 544b such that spring retention element 528b clears past backstop feature 544b. Backstop feature 544b may feature a ramp to guide spring retention element 528b. A distal end of needle carrier assembly 516 and a distal end of holder 522 may be offset from each other at least the same distance a to allow for spring retention element 528b to traverse distally past backstop feature 544b.

It may be appreciated that the frictional force between corresponding contacting surfaces of backstop feature 544b and spring retention element 528b may at least partly determine an amount of force to release spring retention element 528b from backstop feature 544b. This force may allow for lateral deflection of spring retention element 528b and thus allow the expansion of second spring 526. In some examples, the amount of force is at least 0.1 pounds. In some examples, the amount of force is at least 0.5 pounds. In some examples, the amount of force is at least 1 pound. In some examples, the amount of force is at least 2 pounds. In some examples, the amount of force is at least 3 pounds. In some examples, the amount of force is at least 4 pounds. In some examples, the amount of force is at least 5 pounds.

Although the figure shows backstop feature 544b preventing lateral deflection of spring retention element 528b in a radially outward direction, it is contemplated that an inverse structural relationship can be achieved. For instance, the ramped surface of spring retention element 528b can be reversed to face the opposite direction as shown in FIG. 13. Further, the ramped surface of spring retention element 528b may be biased in a radially inward direction by second spring 526 against backstop feature 544b. In such examples, backstop feature 544b may be located radially inward of spring retention element 528b.

Accordingly, in some examples, materials utilized to form holder 522 and needle carrier assembly 516 may be selected based on a desired amount of force to release spring retention element 528b for lateral deflection. Examples of such materials may include polycarbonate, ABS, PC/ABS, polypropylene, HIPS (High impact polystyrene), polybutylene terephthalate (PBT), polyoxymethylene (POM), acetal, polyacetal, polyformaldehyde, PTFE, high density polyethylene (HDPE), ultra-high-molecular-weight polyethylene (UHMWPE), nylon, polyethylene terephthalate (PET), thermoplastic elastomer (TPE), thermoplastic polyurethane (TPU), TPSiv, cyclo-olefin polymer (COP), cyclo-olefin copolymer (COC), and/or liquid-crystal polymer (LCP).

An angle θ of a portion of spring retention element 528b in contact with second spring 526 may also affect the amount of frictional force to laterally deflect spring retention element 528b and so to release second spring 526. Accordingly, the angle θ may be selected based on a desired amount of force to laterally deflect spring retention element 528b sufficiently to release second spring 526. In some examples, the angle θ is at least 1 degree with respect to a vertical axis of the spring retention element 528b. In some examples, the angle θ is at least 5 degrees. In some examples, the angle θ is at least 10 degrees. In some examples, the angle θ is at least 15 degrees. In some examples, the angle θ is at least 20 degrees. In some examples, the angle θ is about 30 to 45 degrees. In addition, the force profile of second spring 526 may affect a target amount of frictional force to laterally deflect spring retention element 528b. Accordingly, in some examples, the force profile of second spring 526 may be taken into account when selecting one or both of the materials for forming holder 522 and needle carrier assembly 516 and the angle θ of the portion of spring retention element 528b in contact with second spring 526.

An angle β of spring retention element 528b with respect to a vertical axis may also affect the amount of frictional force to laterally deflect spring retention element 528b and so to release second spring 526. By contacting spring retention element 528b, second spring 526 may exert a force on spring retention element 528b at a distance d from a bottom of spring retention element 528b that causes a torque moment sufficient to induce a lateral deflection of spring retention element 528b.

FIG. 13 further illustrates needle carrier assembly 516 comprising a deflecting element 546 configured to contact spring retention element 528b and maintain spring retention element 528b in a laterally deflected orientation once second spring 526 has initially deflected spring retention element 528b and sufficiently driven needle carrier assembly 516 in the proximal direction, as will be shown in more detail in FIG. 14. Deflecting element 546 may prevent spring retention element 528b from contacting the windings of second spring 526 while second spring 526 is extending, smoothing the operation of applicator 500 and preventing energy released by second spring 526 and designed for driving needle carrier assembly 516 in the proximal direction from being absorbed by undesired contact with spring retention element 528b during the release of second spring 526.

In some examples, the angle θ of the portion of spring retention element 528b in contact with second spring 526 may be substantially 90° (e.g., flat) and deflecting element 546 may have a ramped or angled surface in contact with spring retention element 528b in the position illustrated in FIG. 13. In such examples, deflecting element 546, in addition to the above-described functionality, may be configured to initially deflect spring retention element 528b as first spring 524 drives holder 522 from the position illustrated in FIG. 13 to the position illustrated in FIG. 14.

In some examples, inner housing 506 may comprise a protrusion 548 extending from inner housing 506 in the distal direction. Protrusion 548 may be configured to contact at least one of spring retention elements 528a, 528b and backstop features 544a, 544b in the pre-activation state such that spring retention elements 528a, 528b are prevented from laterally deflecting until holder 522 and needle carrier assembly 516 have translated at least a predetermined minimum distance in the distal direction. Accordingly, protrusion 548 may provide a measure of drop protection such that applicator 500 may not prematurely fire in response to a concussive shock from being dropped before intentional activation.

Turning back to FIG. 10, inner housing 506 may further comprise an engagement element 550 configured to engage with a protrusion 552 of needle carrier assembly 516 upon needle carrier assembly 516 translating in the distal direction beyond a predetermined threshold, thereby preventing needle carrier assembly 516 from translating in the distal direction beyond the predetermined threshold. It is contemplated that this may ensure needle carrier assembly retraction in the event of an air firing or dry firing in which applicator 500 is somehow activated when not held against the skin of the host. In some examples, the predetermined threshold may correspond to the distal end of needle carrier assembly 516 extending beyond a point proximal to the distal end of inner housing 506, to a point substantially in line with the distal end of inner housing 506 or to a point distal of the distal end of inner housing 506. In some examples, engagement element 550 comprises a hook, a U-shaped structure, a loop, a protrusion, or any other structure capable of engaging with protrusion 552 as described above.

FIG. 11 illustrates applicator 500 after activation, at a beginning of a force retraction feature process at or near the distal insertion position where on-skin sensor assembly 508 may be in contact with the skin of the host. First spring 524 has driven holder 522, needle carrier assembly 516, needle hub 518, insertion element, and on-skin sensor assembly 508, in the distal direction toward the distal insertion position. During proper operation, holder 522 and on-skin sensor assembly 508 should be pressing against the skin of the host. However, FIG. 11 may also illustrate a dry fire condition, where applicator 500 is not properly pressed against the skin of the host before triggering applicator 500. Accordingly, upon first spring 524 driving holder 522 and needle carrier assembly 516 in the distal direction beyond the predetermined threshold, engagement element 550 contacts protrusion 552, which prevents needle carrier assembly 516 from traveling further in the distal direction, while holder 522 is driven sufficiently further in the distal direction such that backstop features 544a, 544b of needle carrier assembly 516 no longer contact spring retention elements 528a, 528b in the distal insertion position, thereby releasing the first end of second spring 526 and initiating retraction even when applicator 500 is dry fired. The insertion force provided by first spring 524 may be sufficient to additionally overcome the frictional force between corresponding contacting surfaces of backstop feature 544b and spring retention element 528b.

Turning to FIG. 14, first spring 524 has driven holder 522, needle carrier assembly 516 and on-skin sensor assembly 508 in the distal direction to the skin of the host. As first spring 524 drives holder 522, needle carrier assembly 516 and on-skin sensor assembly 508 against the skin of the host, the skin provides a counter force to the force generated by first spring 524. The skin may oppose the force of first spring 524 and bias against the distal end of on-skin sensor assembly 508. Because the distal end of holder 522 is offset from the distal end of on-skin sensor assembly 508 as shown in FIG. 13, the counter force provided by the skin is transferred to holder 522 as first spring 524 continues to drive holder 522 towards the skin while on-skin sensor assembly 508 is pressed against the skin. The counter force provided by the skin allows spring retention element 528b to displace past backstop feature 544b. Once spring retention element 528b has cleared distance a past backstop feature 544b, second spring 526 can laterally deflect spring retention element 528b, thereby releasing second spring 526, which drives needle carrier assembly 516 in the proximal direction. Alternatively, as described above in connection with FIG. 13, where the angle θ of the portion of spring retention element 528b in contact with second spring 526 is substantially 90° (e.g., flat), the ramped or angled surface of deflecting element 546 in contact with spring retention element 528b deflects spring retention element 528b sufficiently to release second spring 526, which drives needle carrier assembly 516 in the proximal direction.

In some examples, engagement element 550 may engage protrusion 552 even when applicator 500 is pressed against the skin of a user. In such examples, engagement element 550 engages protrusion 552 as first spring 524 drives holder 522, needle carrier assembly 516, and on-skin sensor assembly 508 against the skin of the host. As explained above, engagement element 550 prevents needle carrier assembly 516 from moving distally when engagement element 550 engages protrusion 552. This allows spring retention elements 528a, 528b to separate away from backstop features 544a, 544b and allow for release of second spring 526. The engagement of engagement element 550 and protrusion 552 may add additional force to the counter force provided by the skin, thus increasing the energy needed to overcome the frictional engagement of spring retention elements 528a, 528b and backstop features 544a, 544b. In some instances, the engagement of engagement element 550 and protrusion 552 provides an immediate impulse force that converts at least some of the initial energy of first spring 524 into energy needed to overcome the frictional engagement of spring retention elements 528a, 528b and backstop features 544a, 544b. It is contemplated that such examples may benefit users with soft skin or higher body fat percentage.

Turning back to FIG. 12, which illustrates applicator 500 during activation, needle carrier assembly 516 is retracted in the proximal direction by second spring 526, as indicated by arrow 554. In FIG. 12, with backstop features 544a, 544b no longer immobilizing spring retention elements 528a, 528b, first end of second spring 526 pushes against spring retention elements 528a, 528b with sufficient force to deflect spring retention elements 528a, 528b in the distal insertion position when on-skin sensor assembly 508 is in contact with skin of the host, allowing second spring 526 to clear spring retention elements 528a, 528b and drive needle carrier assembly 516 in the proximal direction, thereby maintaining needle carrier assembly 516, needle hub 518 (see FIGS. 7-9) and insertion element 520 (see FIGS. 7-9) in a locked, retracted position even in the event of a dry fire.

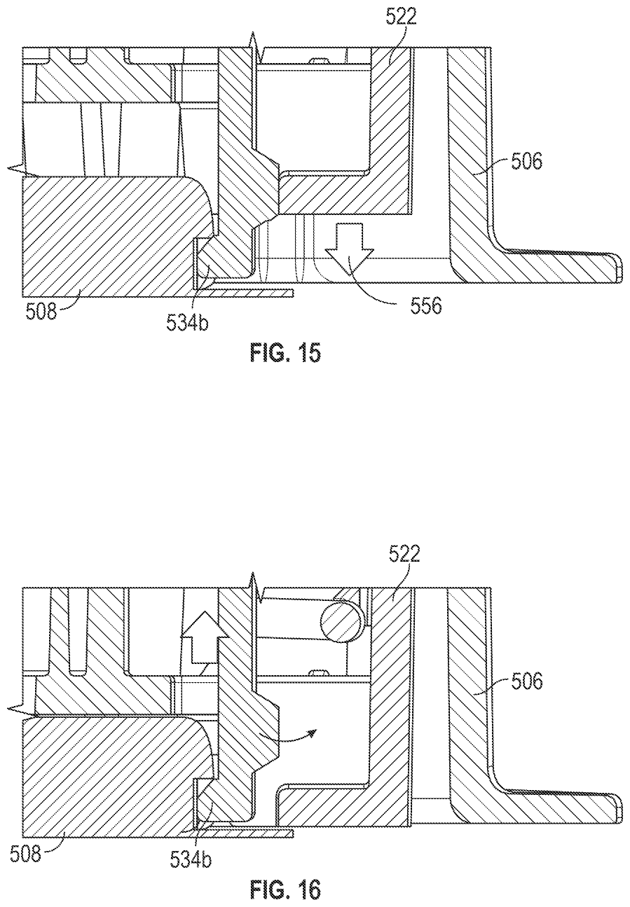

FIGS. 15 and 16 illustrate magnified views of some features of an applicator, such as applicator 500, according to some examples.

In FIG. 15, first spring 524 (see FIGS. 6-12) is driving holder 522, as well as the needle carrier assembly and on-skin sensor assembly 508 in the distal direction, illustrated by arrow 556, toward the distal insertion position. The engagement portion in the form of retention element 534b of the needle carrier assembly is releasably coupled to on-skin sensor assembly 508. As illustrated, during insertion and near the distal inserted position, holder 522 is in contact with spring retention element 534b, preventing spring retention element 534b from deflecting laterally and thereby rigidly securing on-skin sensor assembly 508 to the needle carrier assembly.

In FIG. 16, second spring 526 (see FIGS. 6-12) is driving needle carrier assembly 516 in the proximal direction from the distal insertion position. Because holder 522 has been driven sufficiently in the distal direction, at the distal insertion position, holder 522 is no longer in contact with wearable retention element 534b. Accordingly, wearable retention element 534b is free to deflect laterally, thereby releasing on-skin sensor assembly 508 from wearable retention element 534b and thus from the needle carrier assembly 516. Needle carrier assembly 516 is now driven in the proximal direction by second spring 526, while on-skin sensor assembly 508 is secured to the skin of the host. Moreover, in some examples, because holder 522 is driven to the distal inserted position and substantially held in that position by first spring 524, holder 522 may press against one or both of on-skin sensor assembly 508 or an adhesive patch of on-skin sensor assembly 508, supporting one or both during attachment to the skin of the host.

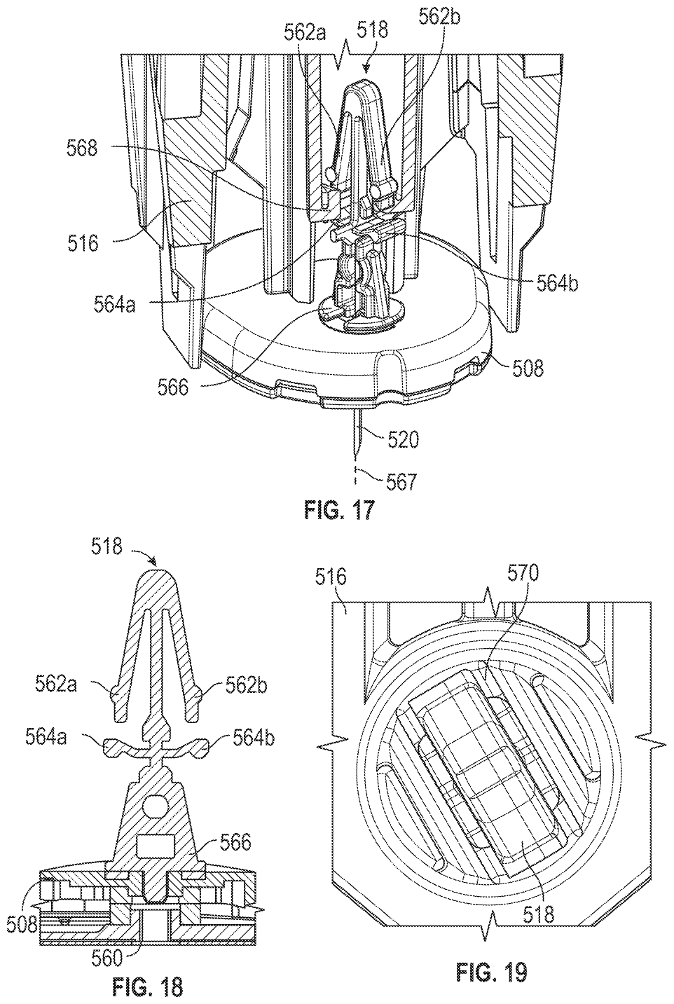

FIG. 17 illustrates a perspective partial cutaway view of needle carrier assembly 516, needle hub 518, and on-skin sensor assembly 508 of applicator 500 of FIGS. 5 and 6, according to some examples. FIG. 18 illustrates a cross-sectional view of needle hub 518 and on-skin sensor assembly 508, according to some examples. FIG. 19 illustrates a top view of a portion of needle carrier assembly 516 and needle hub 518, according to some examples. The following is a description of these features with reference to FIGS. 17-19.

On-skin sensor assembly 508 comprises sensor assembly opening 560. Needle hub 518 is configured to couple insertion element 520 to needle carrier assembly 516 and to substantially maintain a desired orientation of insertion element 520 during insertion of the sensor of on-skin sensor assembly 508 into the skin of the host.

Needle hub 518 comprises a plurality of upper arms 562a, 562b, a plurality of lower arms 564a, 564b, and a base 566. Although two upper arms and two lower arms are illustrated, any number of arms, including a single upper and lower arm, are contemplated. In some examples, upper arms 562a, 562b and lower arms 564a, 564b may be flexible such that, when needle hub 518 is coupled to needle carrier assembly 516, upper arms 562a, 562b and lower arms 564a, 564b secure needle hub 518 in a desired orientation with respect to needle carrier assembly 516. For example, upper arms 562a, 562b may be configured to flex radially inward, such that when disposed through a carrier aperture 568 in needle carrier assembly 516, upper arms 562a, 562b are in contact with an upper surface of needle carrier assembly 516 adjacent to carrier aperture 568 and lower arms 564a, 564b are in contact with a lower surface of needle carrier assembly 516 adjacent to carrier aperture 568. Such an arrangement allows a compliant fit between needle carrier assembly 516 and needle hub 518 where lower arms 564a, 564b deflect to allow upper arms 562a, 562b to expand after clearing surface of carrier aperture 568. The lower arms 564a, 564b can partially or fully relax to bias the needle hub in a distal direction and decrease the clearance between the needle hub and the needle carrier that would otherwise exist with a non-compliant fit. In addition, upper arms 562a, 562b and lower arms 564a, 564b also help to maintain contact between base 566 and a top surface of on-skin sensor assembly 508.

Base 566 comprises an anti-rotation feature. The anti-rotation feature may comprise a key having a shape complementary to at least a portion of sensor assembly opening 560 of on-skin sensor assembly 508 and may be configured to substantially prevent needle hub 518 from rotating about an axis 567 parallel to insertion element 520 with respect to on-skin sensor assembly 508, e.g., to prevent rotation of base 566 within sensor assembly opening 560. In addition, or the alternative, the upper surface of needle carrier assembly 516 adjacent to carrier aperture 568 may comprise a groove 570 configured to accept upper arms 562a, 562b when upper arms 562a, 562b are disposed through carrier aperture 568 in an orientation complementary to an orientation of groove 570, as illustrated in FIG. 19, thereby immobilizing needle hub 518 with respect to needle carrier assembly 516.

In some examples, base 566 further comprises a substantially flat surface configured to mate with a top surface, upper surface, or proximal surface of on-skin sensor assembly 508 and maintain insertion element 520 in a substantially perpendicular orientation to the top surface of on-skin sensor assembly 508, in some cases, when the anti-rotation feature of base 566 is engaged within an opening 560 of on-skin sensor assembly 508.

Based at least upon the above-described features of needle hub 518, on-skin sensor assembly 508, and/or needle carrier assembly 516, base 566 allows easy assembly during manufacture, including but not limited to proper alignment and preassembly of insertion element 520 onto on-skin sensor assembly 508, and/or the ability to easily engage an assembly of needle hub 518, insertion element 520, sensor and on-skin sensor assembly 508 to other portions of assembled applicator 500.

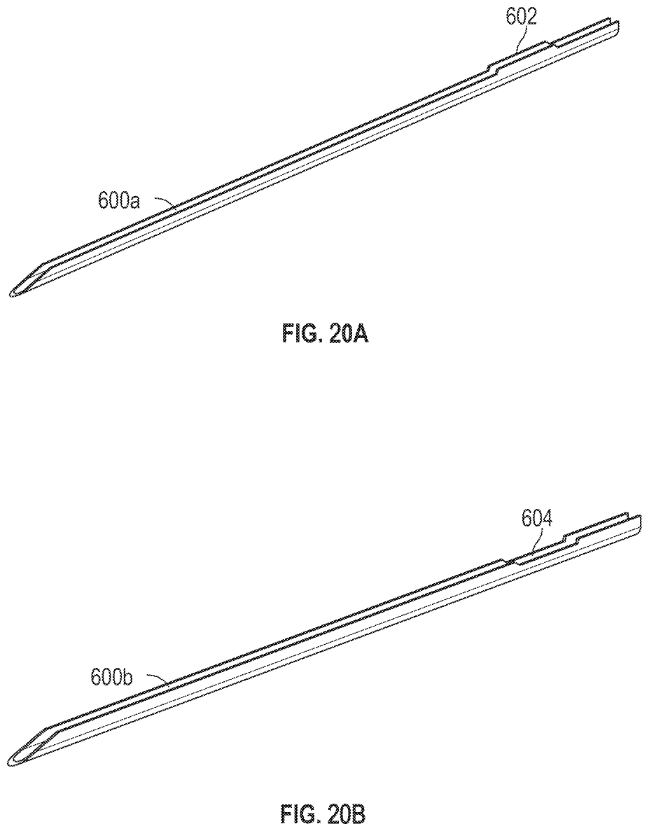

FIGS. 20A and 20B illustrate perspective views of locking features for insertion elements in the form of needles 600a, 600b for use in an applicator for an analyte sensor system, according to some examples. For example, needle 600a of FIG. 20A comprises a locking feature comprising a ridge 602 configured to mate with a complementary-shaped feature within needle hub 518, for example. In the alternative, needle 600b of FIG. 20B comprises a locking feature comprising a groove 604 configured to mate with a complementary-shaped feature within needle hub 518, for example.

In yet another alternative, any insertion element described in this disclosure may comprise a locking feature that heat stakes the selected insertion element to needle hub 518, for example. In yet another alternative, any insertion element described in this disclosure may comprise a locking feature comprising one or more friction-fit or snap-fit elements securing the selected insertion element to needle hub 518, for example. In yet another alternative, any insertion element described in this disclosure may comprise a locking feature comprising complementary clamshell elements on the selected insertion element and needle hub 518, for example, configured to mate with one another. In yet another alternative, any insertion element described in this disclosure may comprise a locking element comprising one or more inserted molded elements configured to couple the selected insertion element to needle hub 518, for example.

During manufacture, applicator 500 may be assembled in stages. For example, and not limitation, if present, first barrier layer 512 may be affixed to inner housing 506. Insertion element 520 may be coupled to needle hub 518, which may then be coupled to on-skin sensor assembly 508. Second spring 526 may be placed into holder 522 or needle carrier assembly 516 and then needle carrier assembly 516 may be disposed into holder 522 and attached to needle hub 518 and to on-skin sensor assembly 508 via wearable retention elements 534a, 534b. First spring 524 may be disposed in holder 522, which may then be installed into inner housing 506. Inner housing 506 may be inserted into and secured to outer housing 504. If present, second barrier layer 530 may be affixed to inner housing 506. If a separate element, activation element 502 may then be disposed into outer housing 504. Any labeling, sterilizing and/or packaging may then be applied to applicator 500.

FIGS. 21-23 illustrate several cross-sectional views, and various features and operating positions, of yet another applicator 700 for an on-skin sensor assembly of an analyte sensor system, according to some examples.

Applicator 700 may include outer applicator housing 504 comprising activation element 502. Outer applicator housing 504 may be configured to translate in a distal direction under force applied by a host of applicator 700, thereby aligning activation element 502 in a position that allows applicator 700 to fire, an alignment illustrated by FIG. 21. As previously described in connection with applicator 500, in some examples, activation element 502 may be disposed in any location, e.g., a top, upper side, lower side, or any other location of applicator 700.

Applicator 700 further comprises inner housing 506, configured to house one or more mechanisms utilized to apply on-skin sensor assembly 508 to skin of a host. Distal surface 510 of a bottom opening of inner housing 506 may define a bottom surface of applicator 700. In some examples, upon pressing applicator 700 against the skin of the host, the skin may deform in a substantially convex shape at distal surface 510 such that at least a portion of a surface of the skin disposed at the bottom opening of applicator housing 506 extends into the bottom opening of inner housing 506, in a proximal direction, beyond a plane defined by distal surface 510.

Although not illustrated in FIGS. 21-23, inner housing 506 may comprise a spring 536 configured to contact outer housing 504 and maintain a predetermined spacing between outer housing 504 and inner housing 506 in the pre-activation orientation (see FIG. 7). Spring 536 may be a compression spring, leaf spring, flex arm spring, a piece of foam or rubber, etc. In some other examples, outer housing 504 may comprise spring 536 and spring 536 may be configured to contact inner housing 506.

Applicator 700 may further comprise a needle carrier assembly 702. Needle carrier assembly 702 comprises an engagement portion, which may be in the form of wearable retention and/or alignment elements 534a, 534b configured to pass through holder 704 and releasably couple on-skin sensor assembly 508 to holder 704 and/or to needle carrier assembly 702. Although two wearable retention and/or alignment elements are illustrated, any number of wearable retention and/or alignment elements are contemplated.

Applicator 700 further comprises needle hub 518 configured to couple insertion element 520 to needle carrier assembly 702. Insertion element 520 is configured to insert sensor of on-skin sensor assembly 508 into skin of the host. In some examples, insertion element 520 comprises a needle, for example, an open sided-needle, a needle with a deflected-tip, a curved needle, a polymer-coated needle, a hypodermic needle, or any other suitable type of needle or structure. In yet other examples, insertion element 520 may be integrally formed with sensor, in which insertion element 520 may be sufficiently rigid to be inserted partially into skin of the host with minimal or no structural support.

Applicator 700 may further include holder 704 releasably coupled to needle carrier assembly 702 and configured to guide on-skin sensor assembly 508 while coupled to needle carrier assembly 702, e.g., at least during translation from a proximal position to a distal insertion position. As previously described in connection with applicator 500, on-skin sensor assembly 508 may be stripped or released from holder 704 and/or needle carrier assembly 702 once on-skin sensor assembly 508 is disposed on the skin of the host.

Applicator 700 may further comprise an insertion assembly configured to translate insertion element 520, needle hub 518, and needle carrier assembly 702 from a proximal position, in the distal direction, to a distal insertion position. Such an insertion assembly may include first spring 524. First spring 524 may be a compression spring, or any suitable type of spring, and may have its first end in contact with or coupled to inner applicator housing 506 and its second end in contact with or coupled to holder 704. First spring 524 is configured to, upon activation of the insertion assembly, translate holder 704, needle carrier assembly 702, needle hub 518, insertion element 520 and on-skin sensor assembly 508, in the distal direction to the distal insertion position. Substantially at the distal insertion position, needle carrier assembly 702 may decouple from holder 704 and on-skin sensor assembly 508.

Applicator 700 may further comprise a retraction assembly configured to translate needle carrier assembly 702, needle hub 518 and insertion element 520, in the proximal direction, from the distal insertion position to a proximal retracted position. In some examples the initial proximal position may be the same as the proximal retracted position. In other examples, the initial proximal position may be different from the proximal retracted position. Such a retraction assembly may include a second spring 706. Second spring 706 may be a compression spring, or any suitable type of spring, and may have a first end contacting or coupled to holder 704 and a second end, comprising a tang 708 (e.g., a spring portion or spring end) disposed substantially along a diameter of second spring 706, in contact with or coupled to a spring retention element 710 of holder 704, at least until retraction. Spring retention element 710 may comprise, e.g., an arm, a deflection element, a tab, a detent, a snap or any other feature capable of a retaining function. Spring retention element 710 may have substantially the same form and function as spring retention elements 528a, 528b of applicator 500 except as described below. Second spring 706 is configured to translate needle carrier assembly 702, needle hub 518, and insertion element 520 in the proximal direction from the distal insertion position to the proximal retracted position. Tang 708 of second spring 706 is released from spring retention element 710 in the distal insertion position when spring retention element 710 is not backed up by backstop element 712 and in response to tang 708 of second spring 706 pushing against spring retention element 710 with a force exceeding a predetermined threshold sufficient to overcome and deflect spring retention element 710.

Needle carrier assembly 702 further comprises a backstop feature 712, configured to prevent lateral motion of spring retention element 710 of holder 704 in at least the proximal pre-activation position, thereby supporting retention of second spring 706 between spring retention element 710 and holder 704 until retraction. In the orientation shown in FIG. 21, second spring 706 is exerting a force against spring retention element 710 but backstop feature 712 prevents lateral deflection of retention element 710.

Holder 704 further comprises needle carrier retention element 542, which may comprise a deflectable arm, rigid arm, deformable feature, snap, catch, or hook. Upon needle carrier assembly 702 reaching the proximal retraction position after activation, needle carrier retention element 542 is configured to engage with needle carrier assembly 702, thereby maintaining needle carrier assembly 702, needle hub 518 and insertion element 520 in a locked, retracted position, limiting access to insertion element 520.

Although not illustrated in FIGS. 21-23, inner housing 506 of applicator 700 may further comprise engagement element 550 and needle carrier assembly 702 may further comprise protrusion 552 and may function substantially as previously described in connection with at least FIGS. 10-12.

Although not illustrated in FIGS. 21-23, inner housing 506 of applicator 700 may further comprise a protrusion extending from inner housing 506 in the distal direction, substantially as previously described protrusion 548. Similar to that previously described in connection with FIG. 13, this protrusion may be configured to contact at least one of spring retention element 710 and backstop feature 712 in the pre-activation state such that spring retention element 710 is prevented from laterally deflecting until holder 704 and needle carrier assembly 702 have translated at least a predetermined minimum distance in the distal direction. Accordingly, the protrusion may provide a measure of drop protection such that applicator 700 may not prematurely fire in response to a concussive shock from being dropped before activation.

Applicator 700 functions substantially similarly to applicator 500 with the exception that instead of utilizing spring retention elements 528a, 528b, which are disposed along an outside of second coil of spring 526 and are configured to contact and retain a coil of second spring 526, applicator 700 utilizes spring retention element 710, which is disposed along an inside of second spring 706 and is configured to contact and retain tang 708 of second spring 706 along a diameter of second spring 706. Disposing spring retention element 710 within and substantially along a center of second spring 706, as opposed to along an outside of second spring 706, further ensures that spring retention element 710 does not contact the coils of second spring 706 as second spring 706 extends during retraction, thereby smoothing the operation of applicator 700. In addition, the arrangement including spring retention element 710, as opposed to spring retention elements 528a, 528b mitigates the risk of, and difficulty ensuring that, multiple spring retention elements trigger or are overcome at substantially the same time.

FIG. 21 illustrates a state of applicator 700 prior to activation, according to some examples. Holder 704, needle carrier assembly 702, needle hub 518, insertion element 520, on-skin sensor assembly 508, first spring 524 and second spring 526 are all shown in pre-activation positions.

Retention element 532 of holder 704 is in contact with inner housing 506, thereby immobilizing holder 704, and therefore also needle carrier assembly 702, needle hub 518, insertion element 520 and on-skin sensor assembly 508, in the pre-activated state.

Backstop feature 712 of needle carrier assembly 702 is in contact with and prevents spring retention element 710 from deflecting laterally, thereby ensuring spring retention element 710 retains tang 708 of second spring 706 in the loaded or pre-activation position shown.

Activation of applicator 700 may include a host pressing applicator 700 against their skin with sufficient force to translate outer housing 504 in a distal direction toward and with respect to inner housing 506 until activation element 502 is aligned with insertion assembly retention element 532 of holder 704, as shown in FIG. 21. Once such an alignment is achieved, a host may initiate activation element 502, thereby deflecting insertion assembly retention element 532 sufficiently to release holder 704 from inner housing 506. In some other examples, applicator 700 may be configured such that activation element 502 may be activated first, but that actual insertion is not triggered until outer housing 504 is translated sufficiently in the distal direction toward and with respect to inner housing 506. In yet other examples, activation element 502 may be biased toward a center of applicator 700 such that activation element 502 need not be explicitly activated by the host but, instead, activation element 502 may be configured to automatically initiate insertion upon outer housing 504 being translated sufficiently in the distal direction toward and with respect to inner housing 506.

FIG. 22 illustrates applicator 700 after activation and during insertion, according to some examples. First spring 524 drives holder 704, and so needle carrier assembly 702, needle hub 518, insertion element 520, and on-skin sensor assembly 508, in the distal direction toward the distal insertion position. FIG. 22 illustrates on-skin sensor assembly 508 in contact with skin of the host but where holder 704 is not yet fully driven, by first spring 524, into contact with on-skin sensor assembly 508 or skin of the host.

In some examples, masses of each of holder 704, needle carrier assembly 702, needle hub 518, insertion element 520, and on-skin sensor assembly 508 may be specifically designed to reduce or substantially eliminate a tendency of needle carrier assembly 702, needle hub 518, insertion element 520, and on-skin sensor assembly 508 to detach from holder 704 while being driven in the distal direction during insertion. In some examples, a force exerted by first spring 524 may further be selected to be sufficient for proper operation of applicator 500, while not so large as to further exacerbate such above-described inertially triggered detachment. In some examples, a spring (not shown) may be configured to exert a force against a portion of needle carrier assembly 702, for example in the distal direction, sufficient to prevent needle carrier assembly 516 from inertially triggered detaching from holder 704 during insertion.

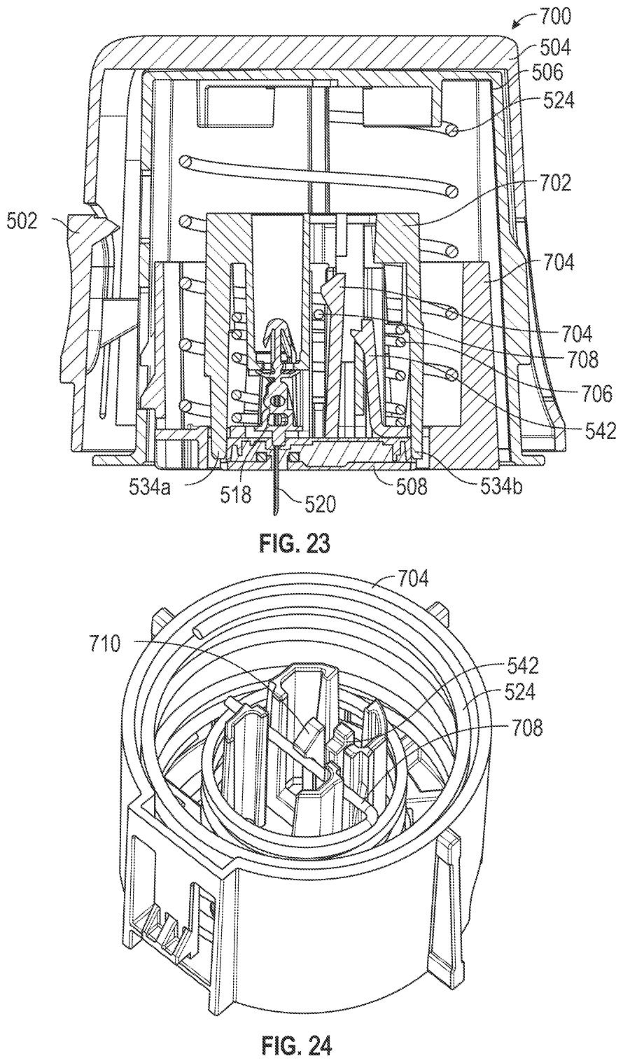

FIG. 23 illustrates applicator 700 after activation and at or near the distal insertion position, according to some examples. First spring 524 has driven holder 704, needle carrier assembly 702 and on-skin sensor assembly 508 in the distal direction to the distal inserted position. Since first spring 524 has driven holder 704 a short distance farther in the distal direction than needle carrier assembly 702, backstop feature 712 is no longer in contact with spring retention element 710, allowing second spring 706 (e.g. tang 708) to laterally deflect spring retention element 710, thereby releasing second spring 706, which drives needle carrier assembly 702 in the proximal direction. Alternatively, similar to that described above in connection with applicator 500 in FIG. 13, where the angle θ of the portion of spring retention element 710 in contact with tang 708 of second spring 526 is substantially 90° (e.g., flat), spring retention element 710 may be biased to automatically deflect sufficiently to release second spring 526 once backstop feature 712 is no longer in contact with spring retention element 710, thereby freeing second spring 706 to drive needle carrier assembly 702 in the proximal direction. Although not shown in FIGS. 21-23, inner housing 506 may further comprise engagement element 550 configured to engage with a protrusion 552 of needle carrier assembly 702, and to function substantially as previously described in connection with at least FIGS. 10-12. In some examples, a stop feature (not shown) may be disposed at a bottom of applicator 700, e.g., on a distal portion of inner housing 506. Such a stop feature may be configured to contact one or more of on-skin sensor assembly 508, needle carrier assembly 702, or holder 704 in the distal insertion position.

Upon release of second spring 706, second spring 706 is configured to drive needle carrier assembly 702, needle hub 518 and insertion element 520, in the proximal direction. Although not shown in FIG. 23, as needle carrier assembly 702 travels to the proximal retracted position, needle carrier retention element 542 may engage with needle carrier assembly 702, thereby retention needle carrier assembly 702, needle hub 518 and insertion element 520, in a locked, retracted position limiting access to insertion element 520.

FIG. 24 illustrates a perspective view of holder 704, first spring 524 and second spring 706 of applicator 700, according to some examples. FIG. 24 illustrates spring retention element 710, retention tang 708 of second spring 706 in an orientation within applicator 700 before retraction.

During manufacture, applicator 700 may be assembled in stages. For example, and not limitation, if present, as previously described in connection with applicator 500, first barrier layer 512 (see FIG. 6) may be affixed to inner housing 506. Insertion element 520 may be coupled to needle hub 518, which may then be coupled to on-skin sensor assembly 508. Second spring may be placed into holder 704 or needle carrier assembly 702 and then needle carrier assembly 702 may be disposed into holder 704 and attached to needle hub 518 and to on-skin sensor assembly via wearable retention elements 534a, 534b. First spring 524 may be disposed in holder 704, which may then be installed into inner housing 506. Inner housing 506 may be inserted into and secured to outer housing 504. If present, as previously described in connection with applicator 500, second barrier layers 530 (see FIG. 6) may be affixed to inner housing 506. If a separate element, activation element 502 may then be disposed into outer housing 504. Any labeling, sterilizing and/or packaging may then be applied to applicator 700.

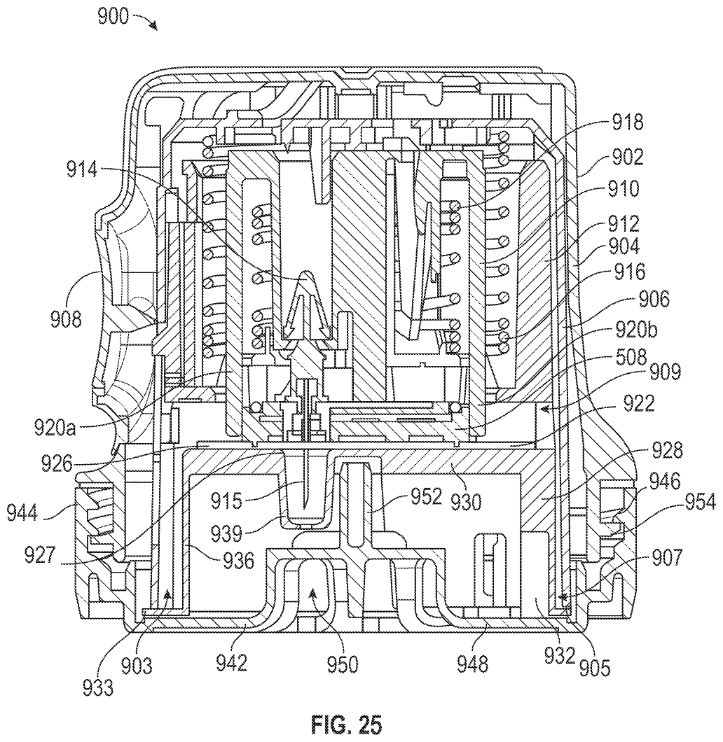

In examples, applicator systems may include a cap and/or a liner removal component. FIG. 25, for example, illustrates an example of an applicator 900 having an applicator housing 902 configured to retain the on-skin wearable medical device, and a deployment mechanism configured to deploy the on-skin wearable medical device to the skin. The applicator housing 902 may be configured similarly as in examples of applicators disclosed herein, including having an outer housing 904 and an inner housing 906 as disclosed in regard to the examples of FIGS. 5-24. The outer housing 904 for example, may be configured similarly as the outer housing 504 and the inner housing may be configured similarly as the inner housing 506. The applicator housing 902 may be configured to be gripped by a user in examples. Various other configurations of applicator housings may be utilized as desired.

The applicator housing 902 may include an internal cavity 903 for retaining the on-skin wearable medical device. The housing 902 may include an opening 905 at an end portion 907 of the internal cavity 903 for the on-skin wearable medical device to be deployed from. The internal cavity 903 may include a proximal end portion 909 that may include the on-skin wearable medical device coupled to a needle carrier assembly.

The deployment mechanism may be configured similarly as other forms of deployment mechanisms disclosed herein. The deployment mechanism may be configured similarly as the deployment mechanisms disclosed in regard to the examples of FIGS. 5-24. For example, the deployment mechanism may include an engagement portion in the form of one or more retention element(s) for retaining the on-skin wearable medical device and releasing the on-skin wearable medical device from the housing 902 to the skin in examples. The deployment mechanism may include an insertion assembly for inserting at least a portion of the on-skin wearable medical device into the skin. The insertion assembly may insert an insertion element (e.g., a needle) into the skin. The deployment mechanism may drive the insertion element to the skin upon the deployment mechanism deploying the on-skin wearable medical device to skin. The deployment mechanism may include a retraction assembly for retracting the insertion element from the skin. Other forms of deployment mechanisms may be utilized in examples as desired.

The applicator 900 may include an activation element 908 that may operate similarly as the activation element 502. The applicator 900 may include a needle carrier assembly 910 that may operate similarly as the needle carrier assembly 516. The applicator 900 may include a holder 912 that may operate similarly as the holder 522. The applicator 900 may include a hub (e.g., a needle hub 914) that may operate similarly as the needle hub 518. The applicator 900 may include an insertion element 915 (e.g., a needle) that may operate similarly as the insertion element 520. The applicator 900 may include springs 916, 918 that may operate similarly as the springs 524, 526 respectively. The applicator 900 may include retention elements 920a, b that may operate similarly as the retention elements 534a, 534b respectively. Additional components of the applicators shown in FIGS. 5-24 may be utilized with the applicator 900. The applicator 900 may operate in a similar manner and provide similar function as the applicators shown in FIGS. 5-24.

The applicator 900 may include a cap 942 that may be positioned at a distal portion of the applicator housing 902 and may cover the distal opening 905 of the internal cavity 903. The cap 942 may include a grip portion 944 on an exterior surface of the cap 942 and an engagement portion 946 on an interior surface of the cap 942. The cap 942 may include a central portion 948 that covers and spans the distal opening 905 of the internal cavity. The cap 942 may comprise an exterior lid for the applicator 900 upon transport and unpackaging of the applicator 900.

The central portion 948 of the cap 942 may include one or more openings 950 that may allow a sterilizing material such as sterilizing gas to pass through, to sterilize internal components of the applicator 900. The central portion 948 may include a central support 952 that may be configured to press against a liner removal component 928 to retain the liner removal component 928 in position. The central support 952 may be configured to rotate upon uncoupling or unscrewing of the cap 942 from the applicator housing 902.

The engagement portion 946 of the cap 942 may comprise threading or another form of engagement portion 946 for engaging a corresponding engagement portion 954 on an exterior surface of the housing 902. The engagement portion 946 may be configured to be rotated relative to the applicator housing 902 to unscrew from the housing 902 and allow for release of the liner removal component 928 from the applicator housing 902.

The applicator 900 may include a liner removal component 928. The liner removal component 928 may be configured to engage a liner 926 positioned on an engaging surface of the patch 922 and remove the liner 926 from the engaging surface of the on-skin wearable medical device upon being withdrawn from the engaging surface of the on-skin wearable medical device. The liner removal component 928 may include an engaging surface 930 for engaging the liner 926. The engaging surface 930 may be a flattened surface that may extend parallel with the liner 926. The engaging surface 930 may include an opening 927 configured to allow the insertion element 915 to pass through. The liner removal component 928 may further include a sheath 939 configured to cover the insertion element 915. The liner removal component 928 may further include a raised portion 936 that may extend from a distal portion 932 of the liner removal component 928. The raised portion 936 may extend axially within the internal cavity 903.

The distal portion 932 of the liner removal component 928 may include a flange 933 for grip by a user to remove the liner removal component 928 from the internal cavity 903 and accordingly remove the liner 926 from the engaging surface of the on-skin wearable medical device. In examples, the flange 933 may be excluded from use.

The liner 926 may be positioned on an engaging surface of the patch in examples. The liner may cover the engaging surface and may protect the engaging surface from damage, deterioration, or other adverse effects. The liner, for example may comprise a sheet of material that covers the engaging surface of the patch. The liner may have a proximal surface contacting the engaging surface of the patch and a distal surface facing opposite the proximal surface. The liner in examples, may be configured to reduce the possibility of an exposed engaging surface from deteriorating or otherwise losing adhesive properties prior to deployment. For example, during a sterilization process using a gas or other sterilizing material, the liner may reduce the possibility of the engaging surface deteriorating. A sterilizing gas may comprise ethylene oxide (EtO) or another form of sterilizing gas as desired. The liner, however, is to be removed from the engaging surface prior to deployment of the on-skin sensor assembly to the skin.

The applicator 900 may be utilized to deploy an on-skin wearable medical device to skin. The on-skin wearable medical device may comprise the on-skin sensor assembly 508 shown in FIG. 6, for example, which may include a housing, an analyte sensor coupled to the housing, an electronics unit, and a patch 922. The on-skin sensor assembly may have forms as shown in FIGS. 2A-4, for example, or other forms as desired.

The cap 942 and the liner removal component 928 may be removed prior to deployment of the on-skin wearable medical device to skin.

Upon activation, an applicator as disclosed herein may insert the analyte sensor into the skin of a host by utilizing an insertion element (such as insertion element 915).

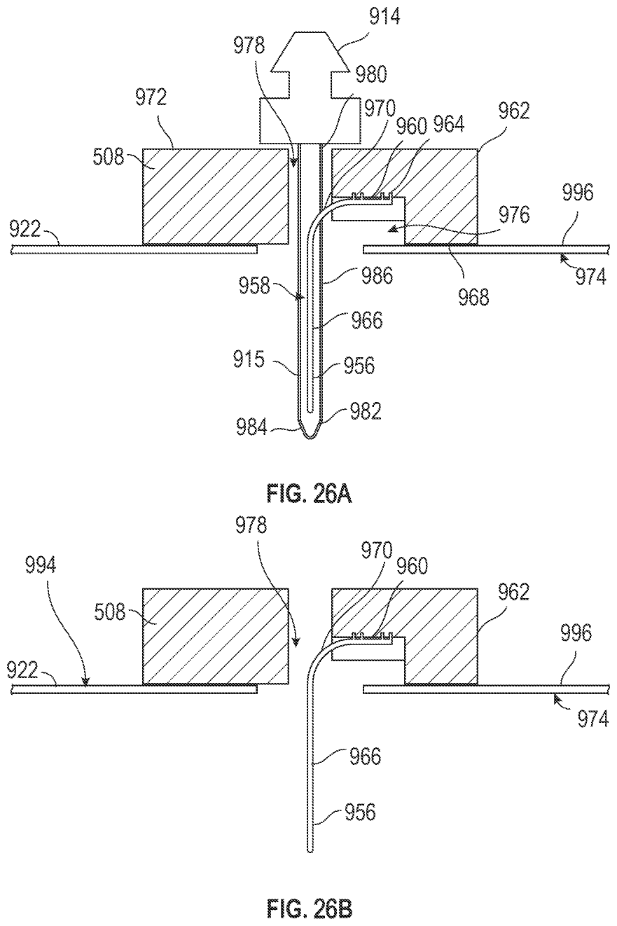

Referring to FIG. 26A, the insertion element 915 may drive the analyte sensor 956 of the on-skin sensor assembly 508 into the host's skin by the analyte sensor 956 extending along a channel 958 of the insertion element 915.

The analyte sensor 956, for example, may include a first portion 960 or contact portion that may be coupled to the housing 962 of the on-skin sensor assembly 508. The first portion 960, for example, may include electrical contacts 964 that may electrically connect to electrical terminals of the on-skin sensor assembly 508 or another component of the on-skin sensor assembly 508. Electrical terminals may be positioned on an interface board or circuit board, or another component of the on-skin sensor assembly 508 as desired. Other methods of coupling between the first portion 960 and the housing 962 may be utilized as desired.

The analyte sensor 956 may include a second portion 966 including a sensing portion that may be configured to be inserted into or through the skin of a host and positioned in or under the skin. The second portion 966, in examples, may extend distally from a distal surface 968 of the housing 962 and may be guided by the insertion element 915 into the skin of the host. The second portion 966 may be straight and may be axially aligned with an opening 978 for the insertion element 915 to pass through, as shown in FIG. 26A.

The analyte sensor 956 may comprise an elongate analyte sensor. The second portion 966 may extend distally to be positioned within the skin layers of the host. In examples, the second portion 966 of the analyte sensor 956 may extend perpendicular with respect to the distal surface 968 of the housing 962. In examples, other angles may be utilized as desired. The second portion 966 may extend perpendicular with respect to the first portion 960 of the analyte sensor 956. In examples, other angles may be utilized as desired.

A bend 970 may angle the second portion 966 of the analyte sensor 956 with respect to the first portion 960 of the analyte sensor 956. The bend 970, for example, may be positioned between the second portion 966 and the first portion 960 and may have a continuous curvature as shown in FIG. 26A or may have another form as desired. The bend 970 may angle the second portion 966 with respect to the first portion 960 at a perpendicular angle or another angle as desired. The bend 970 may be axially aligned with an opening 978 for the insertion element 915 to pass through, as shown in FIG. 26A. Other forms of analyte sensors 956 may be utilized as desired.

The housing 962 of the on-skin sensor assembly 508 may be configured similarly as other forms of housing disclosed herein. The housing 962 may comprise a wearable housing. The housing 962 may be configured to be worn on the skin of the host. The housing 962 may include the distal surface 968, which may be configured to face towards the host's skin. The patch 922 may be positioned on the distal surface 968 of the housing 962. The patch 922 may include the engaging surface 974 for engaging the skin of the host. The engaging surface 974 may comprise an adhesive surface for adhering to the skin of the host in examples or another form of a surface.

The housing 962 may include a proximal surface 972 facing opposite the distal surface 968. The proximal surface 972 may extend parallel with the distal surface 968 or may have another configuration as desired.

The housing 962 may include a cavity 976 that may receive the first portion 960 of the analyte sensor 956 in examples. The cavity 976 may have a variety of forms as desired. For example, the cavity 976 may be configured to retain an adhesive (which may comprise a liquid adhesive or curable adhesive) that may couple the first portion 960 of the analyte sensor 956 to the housing 962 in examples. The cavity 976 may include one or more dams or other features that may retain the adhesive and may be utilized to electrically isolate portions of the analyte sensor 956 from each other if desired. In examples, the cavity 976 may comprise a recess for the first portion 960 of the analyte sensor 956 to be inserted into, to otherwise couple with the housing 962. In examples, use of a cavity 976 may be excluded and the first portion 960 of the analyte sensor 956 may otherwise couple to the housing 962.

The housing 962 may include an opening 978 for the insertion element 915 to pass through. The opening 978 may extend through the proximal surface 972 of the housing 962 and may extend to the distal surface 968 of the housing 962. The opening 978 may be configured for the insertion element 915 to be retracted proximally through from the skin. The insertion element 915 may be retracted following penetration of the host's skin. FIG. 26B, for example, illustrates the insertion element 915 having been retracted from the host's skin. In examples, the insertion element 915 may be positioned within the opening 978 upon insertion into the host's skin or may be passed distally relative to the opening 978 upon insertion into the host's skin. In an example as shown in FIG. 26A, the insertion element 915 may be positioned within the opening 978 and may be static relative to the opening 978 upon insertion into the host's skin. For example, as shown in FIGS. 7-8 and 21-23, the insertion element 915 may move distally along with the housing 962 of the on-skin sensor assembly 508 and may remain static relative to the housing 962 upon insertion into the host's skin. Other forms of insertion may be utilized in examples.

The insertion element 915 may include a proximal end portion 980 and a distal end portion 982 comprising a tip 984 of the insertion element 915. The tip 984 may comprise a sharpened tip in examples, and may be configured to puncture the host's skin and be inserted into the host's skin.

The needle hub 914 may be positioned at the proximal end portion 980 of the insertion element 915. The needle hub 914 may be in contact with the proximal surface 972 of the housing 962 or may be spaced from the proximal surface 972 as desired.

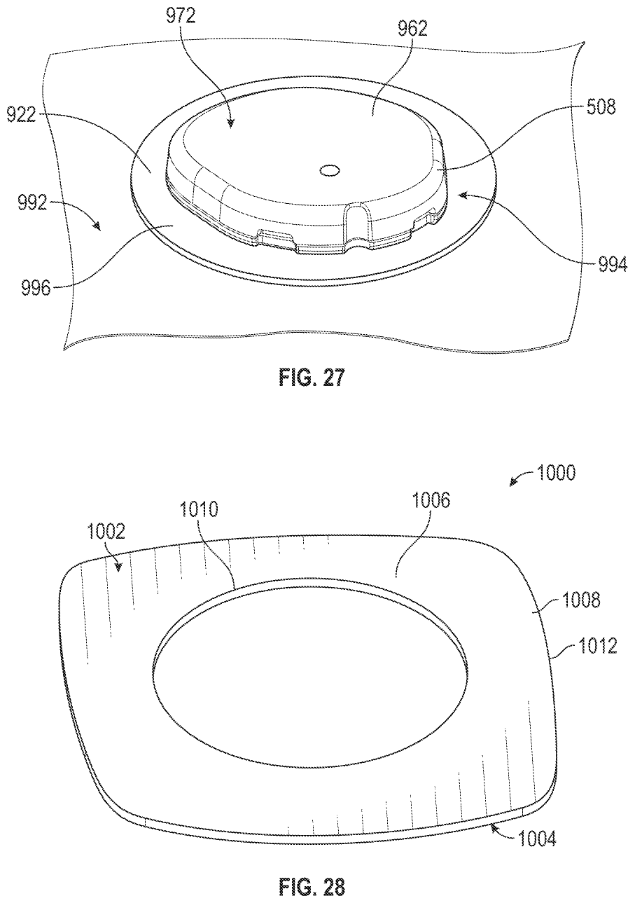

FIG. 27 illustrates the on-skin wearable medical device in the form of on-skin sensor assembly 508 disposed on the skin 992 of a host. The on-skin wearable medical device may comprise a continuous glucose monitoring system in examples. The on-skin wearable housing 962 is shown positioned on the skin 992 with the proximal surface 972 of the housing 962 facing away from the skin 992 and the distal surface 968 (marked in FIG. 26A) of the housing 962 facing towards the skin 992. The distal surface 968 of the housing 962 may be positioned upon a proximal surface 994 or upper surface of the patch 922 that may face opposite the engaging surface 974 or distal surface of the patch 922.

The patch 922 may extend radially outward from the on-skin wearable housing 962 in examples. A portion of the patch 922 that is positioned radially outward of the housing 962 may comprise a skirt portion 996. The proximal surface 994 of the patch 922 at the skirt portion 996 may be exposed, and the proximal surface 994 positioned inward of the skirt portion 996 may be covered by the housing 962.

Referring to FIG. 28, a patch 1000 may be utilized in examples. The patch 1000 may comprise an overlay patch. The overlay patch 1000 may be configured to be applied onto at least a portion of an on-skin wearable medical device. The overlay patch 1000 may be configured to be applied when the on-skin wearable medical device is disposed upon the skin of the host.

FIG. 28 illustrates a perspective view of an example of an overlay patch 1000. The overlay patch may include an upper or proximal surface 1002 and may include a lower, distal, or engaging surface 1004. The lower, distal, or engaging surface 1004 may face opposite the upper or proximal surface 1002.

The lower, distal, or engaging surface 1004 may be configured to engage skin of a host, or at least a portion of an on-skin wearable medical device, to couple the overlay patch 1000 to the respective skin or portion of the on-skin wearable medical device. In examples, the engaging surface 1004 may be configured to engage both skin and at least a portion of the on-skin wearable medical device to couple to both the skin and the portion of the on-skin wearable medical device. In examples, the engaging surface 1004 may comprise an adhesive surface or an adhesive layer that is configured to engage skin and/or a portion of the on-skin wearable medical device.

In examples, the lower, distal, or engaging surface 1004 of the overlay patch 1000 may be configured to engage the proximal surface 994 (marked in FIG. 27) of a second patch 922 or underlay patch. The engaging surface 1004, for example, may comprise an adhesive surface or adhesive layer configured to adhere to at least a portion of the on-skin wearable medical device comprising the proximal surface 994 of the second patch 922. In examples, the adhesive may be configured to adhere to skin 992 of the host as desired. In examples, the engaging surface 1004 may be configured to adhere to a portion of the on-skin wearable medical device comprising the wearable housing 962 (as shown in FIG. 73 for example). In examples, the engaging surface 1004 may be configured to adhere to another portion of the on-skin wearable medical device as desired.

The upper or proximal surface 1002 may comprise a carrier substrate for the lower, distal, or engaging surface 1004 and may comprise an exposed surface of the overlay patch 1000 upon application of the overlay patch 1000. The proximal surface 1002, for example, may be non-adhesive and may be configured to comprise an uppermost surface of the system including the combination of the on-skin wearable medical device and the overlay patch 1000. In examples, the overlay patch 1000 may be configured for additional devices or components to be positioned upon the overlay patch 1000 (e.g., additional overlay patches or other components).

In examples, the lower, distal, or engaging surface 1004 may comprise an adhesive suitable for skin adhesion, for example a pressure sensitive adhesive (e.g., acrylic, rubber-based, or other suitable type). The upper or proximal surface 1002 may comprise a carrier substrate such as spun lace polyester or polyurethane film, or other forms of materials. In examples, other forms of materials may be utilized for the lower, distal, or engaging surface 1004 and/or the upper or proximal surface 1002.

The lower, distal, or engaging surface 1004 may extend in a plane that is parallel with the plane of the upper or proximal surface 1002.

The overlay patch 1000 may be flexible in examples. The flexibility of the overlay patch 1000 may allow the overlay patch 1000 to conform to a shape of the skin of the host or the portion of the on-skin wearable medical device to which the overlay patch 1000 is applied. The overlay patch 1000, for example, may be configured to curve or contour to a shape to which the overlay patch 1000 is applied. In examples, a rigid overlay patch 1000 may be utilized.

The overlay patch 1000 may be made of a fabric or polymer, or other material in examples. The fabric or polymer may be flexible in examples, to allow the overlay patch 1000 to curve or contour to a shape to which the overlay patch 1000 is applied.

The overlay patch 1000 may include an overlap portion 1006 in examples. The overlap portion 1006 may be configured to overlap at least a portion of the on-skin wearable medical device in examples. The overlap portion 1006 may cover at least the portion of the on-skin wearable medical device in examples. As shown in FIG. 28, the overlap portion 1006 may comprise a central portion of the overlay patch 1000 in examples. The overlay patch 1000 may include a skirt portion 1008 in examples. The skirt portion 1008 may be configured to be positioned radially outward of the on-skin wearable medical device or otherwise not overlap with the on-skin wearable medical device in examples. The skirt portion 1008 may be configured to directly couple or adhere to the skin of the host in examples. The skirt portion 1008 may comprise a peripheral portion of the overlay patch 1000 in examples. The skirt portion 1008 may be positioned radially outward of the overlap portion 1006 or the central portion of the overlay patch 1000 as shown in FIG. 28. Other configurations may be provided in examples.

In examples, the configuration of the lower, distal, or engaging surface 1004 of the overlay patch 1000 may differ at the overlap portion 1006 relative to the skirt portion 1008. The skirt portion 1008, for example, may include an engaging surface or adhesive that may be configured for skin adhesion. The overlap portion 1006 may include an engaging surface or adhesive that may be configured for adhesion to the proximal surface 994 of the patch 922 and/or the on-skin wearable housing 962. In examples, the configuration of the lower, distal, or engaging surface 1004 of the overlay patch 1000 may be the same at the overlap portion 1006 and the skirt portion 1008.

The skirt portion 1008 may surround the overlap portion 1006 in examples. The skirt portion 1008, for example, may form a ring about the overlap portion 1006 that may be configured to directly couple or adhere to the skin of the host. Other configurations may be utilized in examples.

The overlay patch 1000 may include one or more openings in examples. The one or more openings may be configured to receive a portion of an on-skin wearable medical device in examples. For example, referring to FIG. 28, the overlay patch 1000 may include a central opening 1010 that may be configured to receive a portion of an on-skin wearable medical device. The central opening 1010 may be configured to receive the on-skin wearable housing 962 (marked in FIG. 27), with the on-skin wearable housing 962 protruding through the central opening 1010. The overlap portion 1006 may surround the central opening 1010 and may be configured to overlap the skirt portion 996 of the patch 922 or underlay patch in examples. The central opening 1010 may be sized with a diameter larger than the diameter of the on-skin wearable housing 962, yet sized to allow the overlap portion 1006 to overlap the patch 922. In examples, other configurations may be provided. For example, FIG. 70 illustrates an example of a patch lacking a central opening.

The overlay patch 1000 may have a ring shape as shown in FIG. 28 for example. The ring shape may be formed by the portion of the overlay patch 1000 surrounding the central opening 1010 in examples. The outer edge 1012 of the overlay patch 1000 may form an outer periphery of the overlay patch 1000 having a rectangular shape, although other shapes (e.g., circular, triangular, among others) may be utilized in examples. Other shapes of overlay patches 1000 may be provided in examples.

The overlay patch 1000 may be sized such that the outer periphery of the overlay patch 1000 has a greater width or diameter than the outer periphery of the patch 922 (or underlay patch). The greater width or diameter may allow the overlay patch 1000 to directly contact and engage skin of the host outside of the width or diameter of the patch 922. In examples, other sizes of overlay patch 1000 may be utilized.

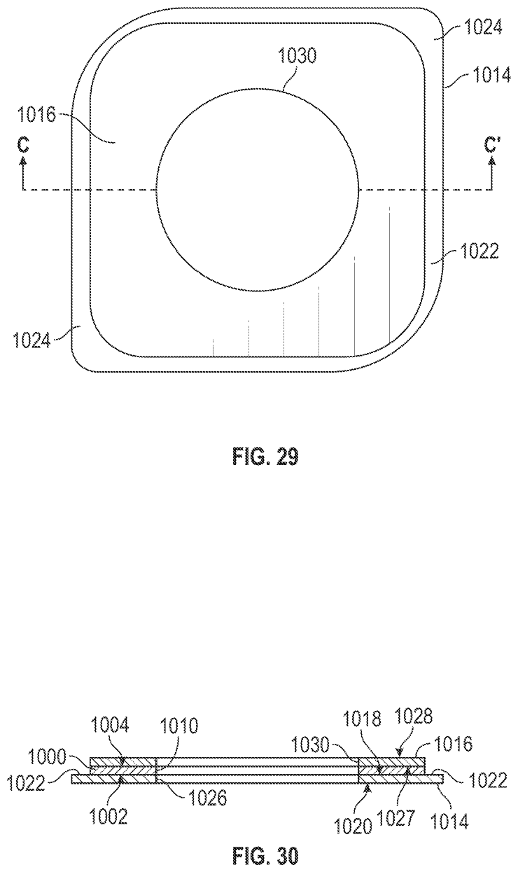

In examples, other components may be utilized with the overlay patch 1000. FIG. 29, for example, illustrates a top view of the overlay patch 1000 (marked in FIG. 30) positioned upon a backing layer 1014. The overlay patch 1000 may be covered with a liner 1016. FIG. 30 illustrates a cross sectional view of the assembly of FIG. 29, along line C-C′ shown in FIG. 29. The overlay patch 1000, backing layer 1014, and liner 1016 may form a patch assembly. Other forms of patch assemblies disclosed herein may be utilized as desired.

The backing layer 1014 or backing may be positioned upon and cover the upper or proximal surface 1002 of the overlay patch 1000. The backing layer 1014, for example, may couple to the upper or proximal surface 1002 of the overlay patch 1000 with an adhesive or other manner of coupling to the overlay patch 1000. Referring to FIG. 30, the backing layer 1014 may include a distal surface 1018 that may comprise an adhesive surface for coupling to the upper or proximal surface 1002 of the overlay patch 1000. In examples, one or more intermediate layers may be utilized to couple the backing layer 1014 to the overlay patch 1000. The backing layer 1014 may include a proximal surface 1020 that may face opposite the distal surface 1018.

Referring to FIG. 29, the backing layer 1014 may protrude radially outward from the outer periphery of the overlay patch 1000 in examples. The backing layer 1014 may have a greater width or diameter than the overlay patch 1000 (and in examples greater than the width or diameter of the liner 1016) and accordingly may form a skirt portion 1022 that extends radially outward from the overlay patch 1000.

In examples, the backing layer 1014 may include one or more retention portions 1024. The retention portions 1024 may be configured for the backing layer 1014 (and correspondingly the overlay patch 1000) to be retained by an applicator for supporting the overlay patch 1000. The retention portions 1024 as shown in FIG. 29 may comprise one or more tabs, yet in examples other forms of retention portions 1024 (e.g., clips, adhesives, pins, clasps, among others) may be utilized as desired. The tabs may be positioned at opposite portions of the backing layer 1014 as shown in FIG. 29, or other positions may be utilized as desired.

The backing layer 1014 may have an outer periphery having a rectangular shape, or other shapes (e.g., circular, triangular, among others) may be utilized in examples. The shape of the outer periphery may correspond to the shape of the overlay patch 1000 in examples, although the shape of the outer periphery may differ as desired.

In examples, the backing layer 1014 may include a central opening 1026 that may match the position of the central opening 1010 of the overlay patch 1000 (as shown in FIG. 30, for example). The central opening 1026 may be configured to receive a portion of an on-skin wearable medical device in a similar manner as the central opening 1010 of the overlay patch 1000. The central opening 1026, for example, may be configured to receive the on-skin wearable housing 962 (marked in FIG. 27).

The backing layer 1014 may be stiffer than the overlay patch 1000 and/or the liner 1016 in examples, and may be made of a more rigid material. In examples, the backing layer 1014 may have a flexibility that is the same as the flexibility of the overlay patch 1000 and/or the liner 1016.

The backing layer 1014 may be configured to be decoupled and removed from the overlay patch 1000 upon the overlay patch 1000 being applied. The adhesive or other form of coupling of the backing layer 1014 to the overlay patch 1000 may be released and the upper or proximal surface 1002 of the overlay patch 1000 may be exposed upon being disposed upon the skin of the host. The backing layer 1014 may be decoupled and removed prior to the overlay patch 1000 being applied, or following an application of the overlay patch 1000 to the skin of the host.

The liner 1016 may be positioned upon and cover the lower, distal, or engaging surface 1004 of the overlay patch 1000. An adhesive of the engaging surface 1004, for example, may couple the liner 1016 to the engaging surface 1004 of the overlay patch 1000.

The liner 1016 may include a proximal surface 1027 that may face towards and couple to the lower, distal, or engaging surface 1004 of the overlay patch 1000. The liner 1016 may include a distal surface 1028 that may face opposite the proximal surface 1027 of the liner 1016.

The liner 1016 may have the same size as the overlay patch 1000 and accordingly may extend no further than the outer periphery of the overlay patch 1000 as shown in FIGS. 29 and 30. In examples, the liner 1016 may have a different size (e.g., greater) than the overlay patch 1000. In examples, the liner 1016 may comprise a plurality of liner components (e.g., a liner having multiple liner sections) that form the liner 1016. The liner 1016 may have a shape that corresponds to a shape of the overlay patch 1000 (e.g., rectangular), although other shapes (e.g., circular, triangular, among others) may be utilized in examples. The shape of the outer periphery of the liner 1016 may correspond to the shape of the overlay patch 1000 in examples, although the shape of the outer periphery of the liner 1016 may differ as desired.

In examples, the liner 1016 may include a central opening 1030 that may match the position of the central opening 1010 of the overlay patch 1000 (as shown in FIG. 30, for example). The central opening 1030 may be configured to receive a portion of an on-skin wearable medical device in a similar manner as the central opening 1010 of the overlay patch 1000. The central opening 1030, for example, may be configured to receive the on-skin wearable housing 962 (marked in FIG. 27).

The liner 1016 may be configured to be decoupled and removed from the overlay patch 1000 upon the overlay patch 1000 being applied. The adhesive or other form of coupling of the liner 1016 to the overlay patch 1000 may be released and the lower, distal, or engaging surface 1004 of the overlay patch 1000 may be exposed prior to being disposed upon the skin of the host. The liner 1016 may be decoupled and removed prior to the overlay patch 1000 being applied.

FIG. 31 illustrates a configuration of the overlay patch 1000 having been applied onto at least a portion of the on-skin wearable medical device (in the form of on-skin sensor assembly 508). The on-skin wearable housing 962 is shown to be positioned within the central opening 1010 of the overlay patch 1000, with the overlay patch 1000 extending radially outward from the on-skin wearable housing 962. The overlap portion 1006 of the overlay patch 1000 is shown to overlap the skirt portion 996 of the patch 922. The skirt portion 1008 of the overlay patch 1000 is shown to extend radially outward from the skirt portion 996 of the patch 922 and to couple directly to the skin 992 of the host.

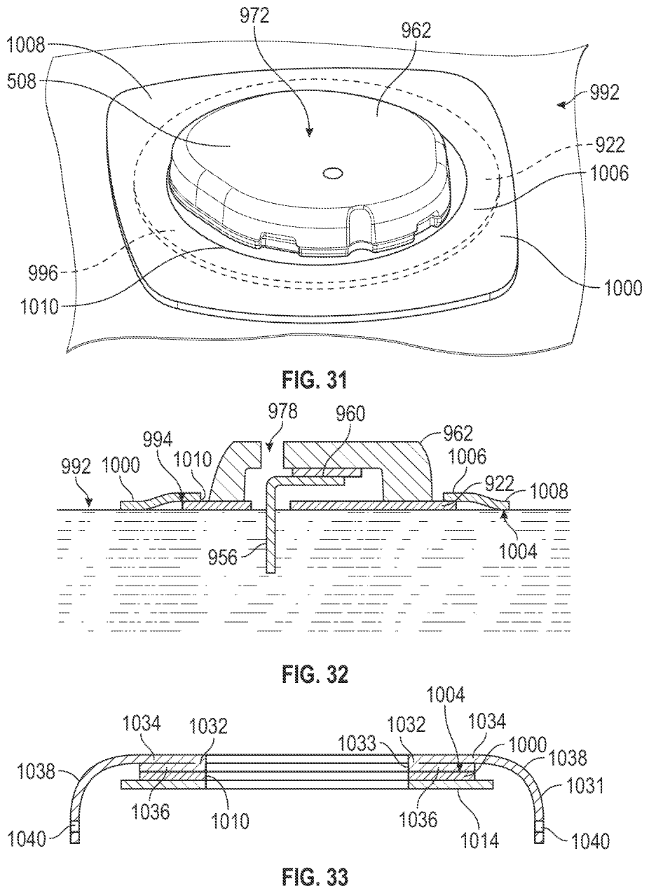

FIG. 32 illustrates a side cross sectional view of the overlay patch 1000 having been applied onto at least the portion of the on-skin wearable medical device (in the form of on-skin sensor assembly 508) in a configuration as shown in FIG. 31. The overlap portion 1006 of the overlay patch 1000 is shown to be positioned upon the proximal surface 994 of the patch 922 (or underlay patch). The engaging surface 1004 of the overlay patch 1000 at the skirt portion 1008 is shown to directly contact the skin 992 of the host.

The overlay patch 1000 may serve to increase the retention of the on-skin wearable medical device to the skin of the host. For example, the additional engagement of the overlay patch 1000 to the skin of the host and to at least a portion of the on-skin wearable medical device may serve to further retain the on-skin wearable medical device to the skin of the host. The overlay patch 1000 may serve to increase the duration that the on-skin wearable medical device may be worn upon the skin of the host. For example, the increased retention to the skin provided by the overlay patch 1000 may allow the on-skin wearable medical device to be worn on skin for a greater period of time, which may reduce the number of times that the on-skin wearable medical device needs to be replaced on the skin, which may reduce cost to the host or other user. The increased retention to the skin provided by the overlay patch 1000 may reduce the effort and labor of a host or other user to frequently replace the on-skin wearable medical device. The increased retention to the skin provided by the overlay patch 1000 may allow the on-skin wearable medical device to be worn on the skin for a greater period of time, which may allow the on-skin wearable medical device to operate for a greater period of time. For example, a longer duration of sensing may be provided by an on-skin sensor assembly, or longer durations of other medical processes (e.g., medicament delivery) may be provided through use of an overlay patch.

Various other configurations of overlay patches may be provided. FIG. 33, for example, illustrates a variation in which a liner 1031 includes one or more fold portions 1032. The fold portions 1032 may allow a first portion 1034 of the liner 1031 to overlap a second portion 1036 of the liner 1031. The first portion 1034, for example, may be positioned distal of the second portion 1036. The fold portions 1032 may comprise a central or inward portion of the liner 1031 in examples. The fold portions 1032 may bound a central opening 1033 of the liner 1031 that may operate in a similar manner as the central opening 1030 of the liner 1016 shown in FIG. 30 for example.

The first portion 1034 may include extension portions 1038 that may extend outward from the overlay patch 1000. The extension portions 1038 may comprise tabs or wings that may extend outward from the overlay patch 1000. Other configurations of extension portions 1038 may be utilized in examples. The extension portions 1038 may include one or more couplers 1040 for utilization in a liner removal assembly that may be configured to remove the liner 1031 from the overlay patch 1000 in examples. The couplers 1040 may comprise openings in the extension portions 1038 or may have another configuration in examples (e.g., clips, adhesives, pins, clasps, among others). The couplers 1040 and extension portions 1038 may be configured such that an outward force applied to the first portion 1034 may pull the first portion 1034 and unfold the one or more fold portions 1032. The unfolding of the one or more fold portions 1032 may pull or peel the second portion 1036 of the liner 1031 off of the lower, distal, or engaging surface 1004 of the overlay patch 1000, thereby exposing the lower, distal, or engaging surface 1004 for application towards the host's skin. Other methods may be utilized to remove the liner 1031 in examples. The liner 1031 may include two or more liner sections in examples, with each extension portion 1038 pulling a respective liner section in examples.

Other configurations of overlay patches may be utilized in examples.

The overlay patch 1000 may provide difficulty for a user to apply to a desired application site. For example, the overlay patch 1000 may need to be aligned with the on-skin wearable medical device to be applied to a desired application site. The central opening 1010, for example, may need to be aligned with the on-skin wearable housing 962 upon application, to allow the on-skin wearable housing 962 to protrude through the central opening 1010. Such alignment may be difficult, particularly if the on-skin wearable medical device is disposed on a host at a position that is difficult to see or access by the host or other user (e.g., on a back surface of an arm or other position that may be difficult to see or access). A user or host may also have difficulty in application of the overlay patch 1000 to avoid the lower, distal, or engaging surface 1004 of the overlay patch 1000 from contacting itself or another portion of the overlay patch 1000 during an application procedure. It may be difficult for a user or host to decouple the engaging surface 1004 from itself or another portion of the overlay patch 1000, which may render the overlay patch 1000 unusable. Other issues may provide difficulty in the application of the overlay patch 1000.

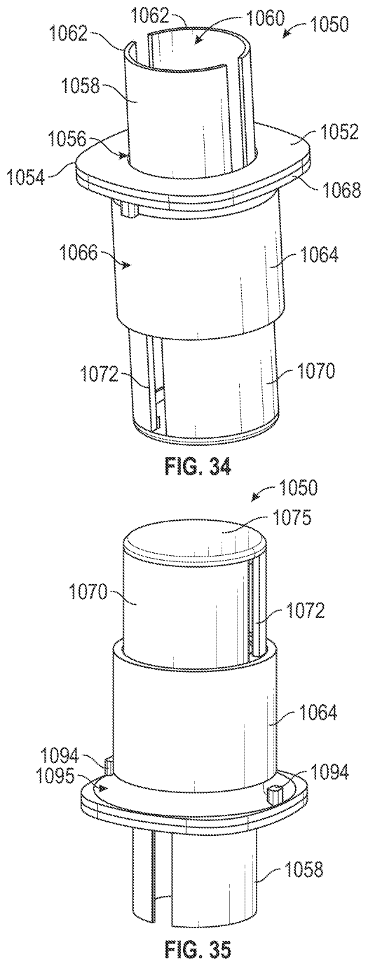

One or more applicators for a patch may be disclosed herein. The applicators may address one or more of the aforementioned issues surrounding application of an overlay patch. FIG. 34, for example, illustrates a perspective view of an applicator 1050 for supporting the patch 1000 and configured to apply the patch 1000 onto at least a portion of an on-skin wearable medical device when the on-skin wearable medical device is disposed on a skin of the host.

The applicator 1050 may include a substrate 1052 for supporting the overlay patch 1000. The substrate 1052, in examples, may comprise a planar surface for the overlay patch 1000 to be positioned upon. The overlay patch 1000 may extend parallel with the planar surface upon being positioned upon the planar surface. In examples, other forms of substrates may be utilized.

The substrate 1052 may include an outer periphery 1054 that may have a diameter or width that is at or greater than the diameter or width of the overlay patch 1000 or the backing layer 1014. The substrate 1052 accordingly may support the full diameter or width of the overlay patch 1000 or backing layer 1014 to the respective outer periphery of the overlay patch 1000 or backing layer 1014. Other configurations may be utilized in examples.

The substrate 1052 may include a central portion 1056. The central portion 1056 may correspond to the position of the central portion or overlap portion 1006 (marked in FIG. 28) of the overlay patch 1000 in examples.

In examples, the applicator 1050 may include an alignment body 1058. The alignment body 1058 may be configured to align the overlay patch 1000 with the on-skin wearable medical device. For example, the alignment body 1058 may orient the applicator 1050 to be positioned at a desired location relative to the on-skin wearable medical device upon application of the overlay patch 1000. The alignment body 1058 may be configured to align with the on-skin wearable housing 962 (marked in FIG. 27) in examples. The alignment body 1058 may center the overlay patch 1000 relative to the on-skin wearable housing 962. The overlay patch 1000 accordingly may be disposed to the on-skin wearable medical device with the central opening 1010 (marked in FIG. 31) of the overlay patch 1000 surrounding the on-skin wearable housing 962. A configuration as shown in FIG. 31 accordingly may result.

The alignment body 1058 may extend from the central portion 1056 of the substrate 1052 in examples. Other positions of the alignment body may be utilized in examples. An intersection of the alignment body 1058 and the substrate 1052 may comprise a displacement portion of the applicator 1050 that may be configured to allow the alignment body 1058 to displace relative to the substrate 1052. The alignment body 1058 may comprise a central portion of the applicator.

In examples, the alignment body 1058 may comprise a protrusion. The protrusion may be configured to extend towards the skin of the host upon the applicator 1050 applying the overlay patch 1000 onto at least a portion of the on-skin wearable medical device. The protrusion, in examples, may include a central opening 1060 that may be configured to receive a portion of the on-skin wearable medical device. The central opening 1060, for example, may receive the on-skin wearable housing 962. One or more walls 1062 of the protrusion may bound the central opening 1060 and may guide the alignment body 1058 into position around the on-skin wearable housing 962. The protrusion, for example, may comprise a hollow cylinder with the central opening 1060 comprising a channel for receiving a portion of the on-skin wearable medical device (e.g., the on-skin wearable housing 962). The walls 1062 of the protrusion may contact or abut the on-skin wearable housing 962 to align the alignment body 1058 relative to the on-skin wearable medical device. The overlay patch 1000 accordingly may be aligned with the on-skin wearable medical device.

In examples, the alignment body 1058 may be configured to displace relative to the substrate 1052. For example, the alignment body 1058 may be configured to retract from the skin of the host upon the applicator 1050 applying the overlay patch 1000 onto at least a portion of the on-skin wearable medical device. The alignment body 1058 may be configured to slide relative to the substrate 1052 in examples.

The alignment body 1058, for example, may be coupled to a first displacement body 1064 of the applicator 1050. The first displacement body 1064 may comprise a sleeve or shroud that may comprise an outer surface 1066 of the applicator 1050. The outer surface 1066 may comprise a grip surface or other form of surface for a user to grip upon utilizing the applicator 1050. The alignment body 1058 may extend distally from the first displacement body 1064. The first displacement body 1064 may include a support plate 1068 for supporting the substrate 1052 in examples.

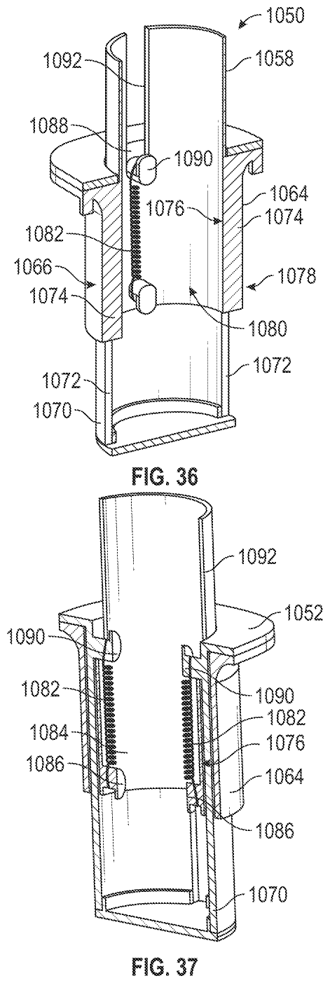

The substrate 1052 may be coupled to a second displacement body 1070 of the applicator 1050. The second displacement body 1070, for example, may comprise a plunger. The plunger may be configured to be operated by a user to displace the alignment body 1058 relative to the substrate 1052. The plunger may be pressed distally relative to the first displacement body 1064 to cause the second displacement body 1070 to slide relative to the first displacement body 1064 and accordingly slide the substrate 1052 relative to the alignment body 1058. The second displacement body may include one or more channels 1072 that a connecting portion 1074 (marked in FIG. 36) of the first displacement body 1064 may pass through to connect the alignment body 1058 with the outer surface 1066 of the first displacement body 1064.

FIG. 35 illustrates a proximal perspective view of the applicator 1050. The second displacement body 1070 may include a pressing surface 1075 for a user to press to displace the second displacement body 1070 relative to the first displacement body 1064.

FIG. 36 illustrates a cross sectional view of the applicator 1050. FIG. 37 illustrates a cross sectional view of the applicator 1050 at a view orthogonal to the view shown in FIG. 36. The first displacement body 1064 is shown to include an inner portion 1076 and an outer portion 1078. The inner portion 1076 comprises a sleeve or shroud that extends to the alignment body 1058. The inner portion 1076 is positioned interior of the second displacement body 1070, within a cavity 1080 or central channel of the second displacement body 1070. The connecting portion 1074 of the first displacement body 1064 may pass through the one or more channels 1072 of the second displacement body 1070 to connect with the outer portion 1078 of the first displacement body 1064. The outer portion 1078 of the first displacement body 1064 may include the outer surface 1066. The outer portion 1078 of the first displacement body 1064 may comprise a sleeve or shroud that extends around the second displacement body 1070.

Referring to FIG. 37, the second displacement body 1070 may comprise a sleeve or shroud extending over the inner portion 1076 of the first displacement body 1064. An end of the sleeve or shroud may comprise the substrate 1052 flaring radially outward from the sleeve or shroud.

The applicator 1050 may comprise a multi-housing or two housing applicator 1050, with a first housing comprising the first displacement body 1064 and the alignment body 1058, and a second housing comprising the second displacement body 1070 and the substrate 1052.

In examples, one or more springs 1082 may be configured to bias the alignment body 1058 relative to the substrate 1052. Referring to FIGS. 36 and 37 for example, the springs 1082 may bias the alignment body 1058 to protrude relative to the substrate 1052. The springs 1082 may bias the alignment body 1058 to be raised relative to the substrate 1052. The bias may be overcome with a distal force upon the second displacement body 1070. The springs 1082 may comprise coil springs as shown in FIGS. 36 and 37 or may comprise another form of spring as desired.

Referring to FIGS. 36 and 37, an interior surface 1084 of the first displacement body 1064 may include one or more spring couplers 1086. An interior surface 1088 of the second displacement body 1070 may include one or more spring couplers 1090. The spring couplers 1086, 1090 may couple to respective ends of the springs 1082 to bias the alignment body 1058 to protrude relative to the substrate 1052. The alignment body 1058 may include one or more channels 1092 for the spring couplers 1090 to slide along during movement of the alignment body 1058 relative to the substrate 1052. In examples, other forms of springs or biasing members may be utilized as desired. In examples, the use of one or more springs may be excluded.

The applicator 1050 may include a liner removal assembly in examples. The liner removal assembly may be configured to remove a liner from the overlay patch 1000 in examples. The liner removal assembly may have a variety of forms in examples. Referring to FIG. 35 for example, the applicator 1050 may include one or more liner couplers 1094. The liner couplers 1094 may be configured to couple to a portion of a liner. For example, referring to FIG. 33, the liner couplers 1094 may couple to the couplers 1040 of the liner 1031. The liner couplers 1094, for example, may comprise protrusions that extend through the couplers 1040 comprising openings in the liner 1031. Other configurations (e.g., the liner couplers 1094 comprising openings, and the couplers 1040 comprising protrusions, among others) may be utilized as desired. The liner couplers 1094 may be positioned upon a proximal surface 1095 of the support plate 1068. The liner couplers 1094 may hold the extension portions 1038 (marked in FIG. 33) of the liner 1031 in place as the overlay patch 1000 is advanced distally, to allow the liner 1031 to remove from or peel off of the overlay patch 1000. The liner removal assembly may be configured to couple to the liner 1031 of the overlay patch 1000 and automatically remove the liner 1031 from the overlay patch 1000 during an application procedure of the overlay patch 1000 onto at least a portion of the on-skin wearable medical device.

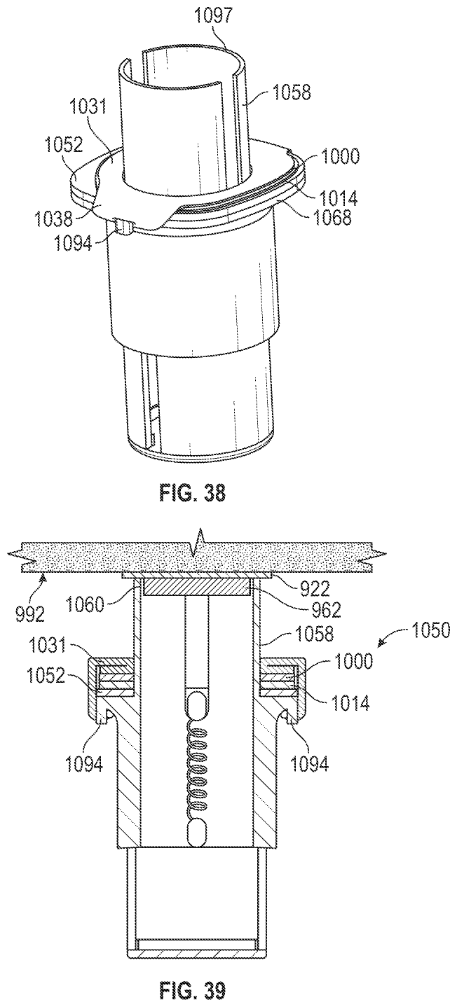

FIG. 38, for example, illustrates the overlay patch 1000, the liner 1031, and the backing layer 1014 positioned upon the substrate 1052. The alignment body 1058 extends through the central openings 1010, 1026, 1033 of the respective overlay patch 1000, backing layer 1014, and the liner 1031. The alignment body 1058 may align the overlay patch 1000 upon the substrate 1052 (e.g., guide the overlay patch 1000 to a defined position upon the substrate 1052).

The extension portions 1038 may extend proximally to couple to the liner couplers 1094. The extension portions 1038 may wrap about the outer periphery of the substrate 1052 and the outer periphery of the support plate 1068 to couple to the liner couplers 1094.

The overlay patch 1000 may be positioned upon the substrate 1052 with the lower, distal, or engaging surface 1004 facing away from the substrate 1052 and in a distal direction towards the skin of the host.

FIGS. 39-41 illustrate an exemplary application procedure of the overlay patch 1000 utilizing the applicator 1050. Steps of the application procedure may be varied, excluded, or substituted as desired. Combinations of features of procedures may be used in combination or substitution as desired.

Referring to FIG. 39, a cross sectional view of the overlay patch 1000, the liner 1031, and the backing layer 1014 positioned upon the substrate 1052 is shown. A perspective view of such a configuration is shown in FIG. 38. A user (such as the host) may position such components upon the substrate 1052 with the couplers 1040 of the liner 1031 shown in FIG. 33 coupled to the liner couplers 1094. The substrate 1052 may be in a retracted or proximal position relative to the alignment body 1058.

The on-skin wearable medical device is shown disposed upon skin 992 of the host. The application of the on-skin wearable medical device to the skin 992 may occur through use of an applicator as disclosed herein or via another method as desired.

The alignment body 1058 may be utilized to align the overlay patch 1000 with the on-skin wearable medical device. For example, as shown in FIG. 39, the on-skin wearable housing 962 may be positioned within the central opening 1060 of the alignment body 1058 to align the overlay patch 1000 with the on-skin wearable medical device. A leading edge 1097 (marked in FIG. 38) of the alignment body 1058 may be positioned upon the patch 922 or underlay patch surrounding the on-skin wearable housing 962. The alignment body 1058 may guide the applicator 1050 to a desired position. A tactile indication of the position of the alignment body 1058 relative to the on-skin wearable medical device may be provided by a feel of the on-skin wearable housing 962 being inserted into the central opening 1060. Such tactile indication may be beneficial if the on-skin wearable medical device is in a position that is difficult to visualize (e.g., on a back surface of an arm or other position).

Referring to FIG. 40, the second displacement body 1070 or plunger may be pressed in a direction towards the skin 992 of the host. The movement of the second displacement body 1070 relative to the first displacement body 1064 may cause the substrate 1052 to displace distally relative to the alignment body 1058. The overlay patch 1000 may advance distally towards the on-skin wearable medical device and the skin 992 of the host.

The substrate 1052 may slide distally away from the support plate 1068. The couplers 1040 accordingly may be pulled by the liner couplers 1094 to cause the liner 1031 to automatically remove from the overlay patch 1000 during the application procedure. The liner 1031 may unfold and peel or otherwise be removed from the lower, distal, or engaging surface 1004 of the overlay patch 1000. The lower, distal, or engaging surface 1004 accordingly may be exposed for application to at least a portion of the on-skin wearable medical device.

Referring to FIG. 41, the second displacement body 1070 or plunger may continue to be pressed in a direction towards the skin 992 of the host until the overlay patch 1000 contacts at least a portion of the on-skin wearable medical device and/or the skin 992. The overlay patch 1000, for example, may have a position as shown in FIG. 31 upon application. The overlay patch 1000 may be positioned upon the patch 922 or underlay patch of the on-skin wearable medical device, or on another portion of the on-skin wearable medical device as desired. A portion of the overlay patch 1000 may be positioned radially outward of the patch 922 or underlay patch for application directly to the skin 992 of the host.

The applicator 1050 may be removed or withdrawn from the skin. The overlay patch 1000 may separate from the applicator 1050 upon removal of the applicator 1050 due to the adhesion or other engagement between the overlay patch 1000 and the on-skin wearable medical device or skin.

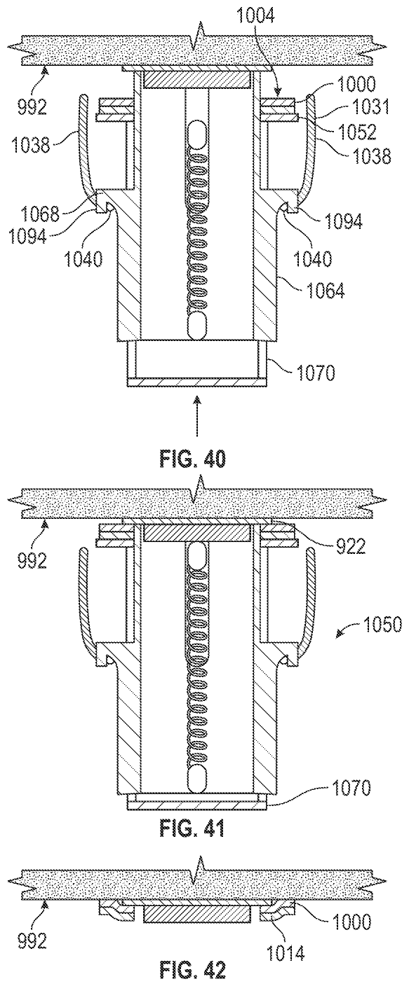

Referring to FIG. 42, upon removal of the applicator 1050, the overlay patch 1000 may be pressed or smoothed by a user (such as the host) to further apply the overlay patch 1000 to the on-skin wearable medical device or the skin 992. A pressure applied by a user may allow the overlay patch 1000 to engage the skin positioned outward of the patch 922 or underlay patch. A pressure applied by the user may further engage the overlay patch 1000 to the on-skin wearable medical device.

In examples, the backing layer 1014 may remain on the overlay patch 1000 and may be removed by the user at this point. In examples, the use of a backing layer 1014 may be excluded, or a backing layer 1014 may be removed prior to positioning of the overlay patch 1000 upon the substrate 1052 of the applicator 1050.

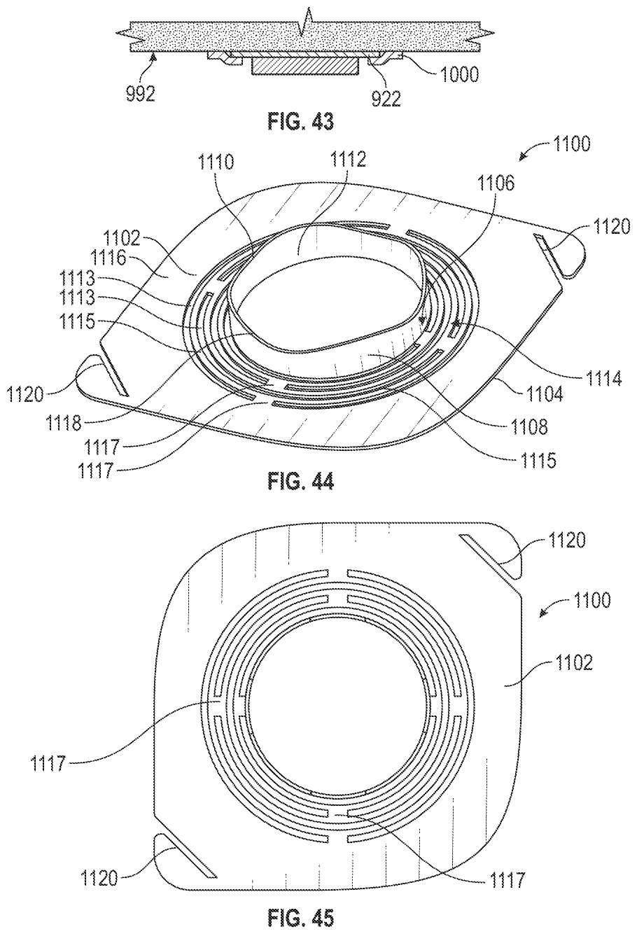

FIG. 43, for example, illustrates a resulting configuration of the overlay patch 1000 applied onto the patch 922 or underlay patch of the on-skin wearable medical device, and applied to the skin 992 of the host. A perspective view of the resulting configuration may be shown in FIG. 31.

The use of the applicator may beneficially improve the ease upon which the overlay patch may be applied to a desired application site.

The features of the examples of FIGS. 34-43 may be utilized solely or in combination with any other example disclosed herein.

Various other configurations of applicators may be utilized in examples.

FIG. 44, for example, illustrates a perspective view of an applicator 1100 for supporting the patch 1000 and configured to apply the patch 1000 onto at least a portion of an on-skin wearable medical device when the on-skin wearable medical device is disposed on a skin of the host.

The applicator 1100 may include a substrate 1102 for supporting the overlay patch 1000. The substrate 1102 may provide a similar function as the substrate 1052 shown in FIG. 34 for example. The substrate 1102, in examples, may comprise a planar surface for the overlay patch 1000 to be positioned upon. The overlay patch 1000 (marked in FIG. 28) may extend parallel with the planar surface upon being positioned upon the planar surface. In examples, other forms of substrates may be utilized.

The substrate 1102 may include an outer periphery 1104 that may have a diameter or width that is at or greater than the diameter or width of the overlay patch 1000 or the backing layer 1014. The substrate 1102 accordingly may support the full diameter or width of the overlay patch 1000 or backing layer 1014 to the respective outer periphery of the overlay patch 1000 or backing layer 1014. Other configurations may be utilized in examples.

The substrate 1102 may include a central portion 1106. The central portion 1106 may correspond to the position of the central portion or overlap portion 1006 (marked in FIG. 28) of the overlay patch 1000 in examples.

In examples, the applicator 1100 may include an alignment body 1108. The alignment body 1108 may provide a similar function as the alignment body 1058 shown in FIG. 34 for example. The alignment body 1108 may be configured to align the overlay patch 1000 with the on-skin wearable medical device. For example, the alignment body 1108 may orient the applicator 1100 to be positioned at a desired location relative to the on-skin wearable medical device upon application of the overlay patch 1000. The alignment body 1108 may be configured to align with the on-skin wearable housing 962 (marked in FIG. 27) in examples. The alignment body 1108 may center the overlay patch 1000 relative to the on-skin wearable housing 962. The overlay patch 1000 accordingly may be disposed to the on-skin wearable medical device with the central opening 1010 (marked in FIG. 31) of the overlay patch 1000 surrounding the on-skin wearable housing 962. A configuration as shown in FIG. 31 accordingly may result.

The alignment body may extend from the central portion 1106 of the substrate 1102 in examples. Other positions of the alignment body may be utilized in examples.

In examples, the alignment body 1108 may comprise a protrusion. The protrusion may be configured to extend towards the skin of the host upon the applicator 1100 applying the overlay patch 1000 onto at least a portion of the on-skin wearable medical device. The protrusion, in examples, may include a central opening 1110 that may be configured to receive a portion of the on-skin wearable medical device. The central opening 1110, for example, may receive the on-skin wearable housing 962. One or more walls 1112 of the protrusion may bound the central opening 1110 and may guide the alignment body 1108 into position around the on-skin wearable housing 962. The protrusion, for example, may comprise a hollow cylinder with the central opening 1110 comprising a channel for receiving a portion of the on-skin wearable medical device (e.g., the on-skin wearable housing 962). The walls 1112 of the protrusion may contact or abut the on-skin wearable housing 962 to align the alignment body 1108 relative to the on-skin wearable medical device. The overlay patch 1000 accordingly may be aligned with the on-skin wearable medical device.

In examples, the alignment body 1108 may be configured to displace relative to the substrate 1102. For example, the alignment body 1108 may be configured to retract from the skin of the host upon the applicator 1100 applying the overlay patch 1000 onto at least a portion of the on-skin wearable medical device. The alignment body 1108 may be configured to retract proximally relative to the substrate 1102 upon movement of the substrate 1102 distally towards the skin of the host.

In examples, the applicator 1100 may include a displacement portion 1114 that may allow a portion of the applicator 1100 (e.g., the alignment body 1108) to displace relative to another portion of the applicator 1100 (e.g., the substrate 1102). The alignment body 1108 may comprise a central portion of the applicator 1100 that is configured to displace relative to the substrate 1102.

The displacement portion 1114 may comprise a portion of the substrate 1102 that is more flexible than the outer portion 1116 of the substrate 1102. The displacement portion 1114, for example, may comprise one or more voids 1113 in the substrate 1102 that may reduce the rigidity of the substrate 1102. The voids 1113 may comprise cut outs or recesses in the substrate 1102 that may reduce material of the substrate 1102 and accordingly reduce the rigidity of the substrate 1102. In examples, the displacement portion 1114 may include a plurality of connectors 1117 that may join portions of the substrate 1102 separated by the voids 1113.

In examples, the displacement portion 1114 may include a plurality of voids 1113 and connectors 1117 of the substrate 1102 that may increase the flexibility of the substrate 1102 at the displacement portion 1114. The voids 1113 and connectors 1117 may be in a pattern, such as a circumferential ring pattern as shown in FIG. 44. Other forms of patterns may be utilized as desired.

The voids 1113 may comprise one or more arcs encircling the alignment body 1108. Rows of arcs may be spaced radially inward from each other. The connectors 1117 may further comprise one or more arcs 1115 of the substrate encircling the alignment body 1108. Rows of the arcs 1115 may be spaced radially inward from each other. One or radially aligned connectors 1117 may connect the adjacent rows of the arcs 1115 to each other. Positions of radially aligned connectors 1117 may be offset circumferentially from each other (e.g., by 90 degrees as shown in FIG. 45) and may alternate in circumferential position as desired (as shown in FIG. 45 for example). Other forms of patterns may be utilized as desired.

In examples, the displacement portion 1114 may comprise a spring. The spring may be formed by the plurality of voids 1113 and connectors of the substrate 1102 and may be configured to bias the alignment body 1108 relative to the substrate 1102. The spring may comprise a flat spring as shown in FIG. 44. The spring may bias the alignment body 1108 to protrude relative to the substrate 1102. The spring may be configured to bias the alignment body 1108 to be raised relative to the substrate 1102. The bias may be overcome by the user applying a distal force to the substrate 1102 (as represented in FIG. 48 for example).

The displacement portion 1114 may be at the central portion 1106 of the substrate 1102 in examples. The outer portion 1116 of the substrate 1102 positioned radially outward of the displacement portion 1114 may be solid or may lack voids and connectors in examples. The outer portion 1116 may be relatively rigid compared to the displacement portion 1114.

In examples, the substrate 1102 may be flexible. For example, the substrate 1102 may comprise a flexible polymer or other material that allows the substrate 1102 (including the displacement portion 1114 and the outer portion 1116) to flex as desired. The flexibility may allow for improved contour to a portion of a host's body upon application to an application site.

The substrate 1102 may comprise a disk in examples, with the alignment body 1108 comprising a cylinder extending from the disk.

In examples, the alignment body 1108 may have a leading edge 1118 that is configured to face towards the skin of the host upon the applicator 1100 applying the overlay patch 1000 onto a portion of the on-skin wearable medical device. The leading edge 1118 may have a varied height as shown in FIG. 44. The leading edge 1118 may have an undulating height in examples (as represented in FIG. 44 for example). The undulating height may be utilized to allow the applicator 1100 to accommodate a size of other components that may be packaged with the applicator 1100. For example, the undulating height may contour to a cylindrical shape of an applicator housing 902 (as marked in FIG. 59 for example) that may be positioned in packaging with the applicator 1100. The undulating height of the leading edge 1118 may curve to allow the leading edge 1118 to be pressed against a cylindrically curved outer surface of the applicator housing 902 upon shipment and transport (e.g., within a box or carton). Such a feature may allow for improved packaging of the applicator 1100. In examples, an undulating height of the leading edge 1118 may allow for improved contact with the wearable housing or patch.

In examples, the applicator 1100 may include one or more retainers 1120 that may be configured to retain the overlay patch 1000 to the applicator 1100. The retainers 1120 may have a variety of forms in examples. The retainers 1120, for example, may comprise one or more slots in the substrate 1102 as shown in FIG. 44 for example. Other forms of retainers may be utilized. In examples, the overlay patch 1000 may be pre adhered to any applicator disclosed herein such that separate retainers are not utilized.

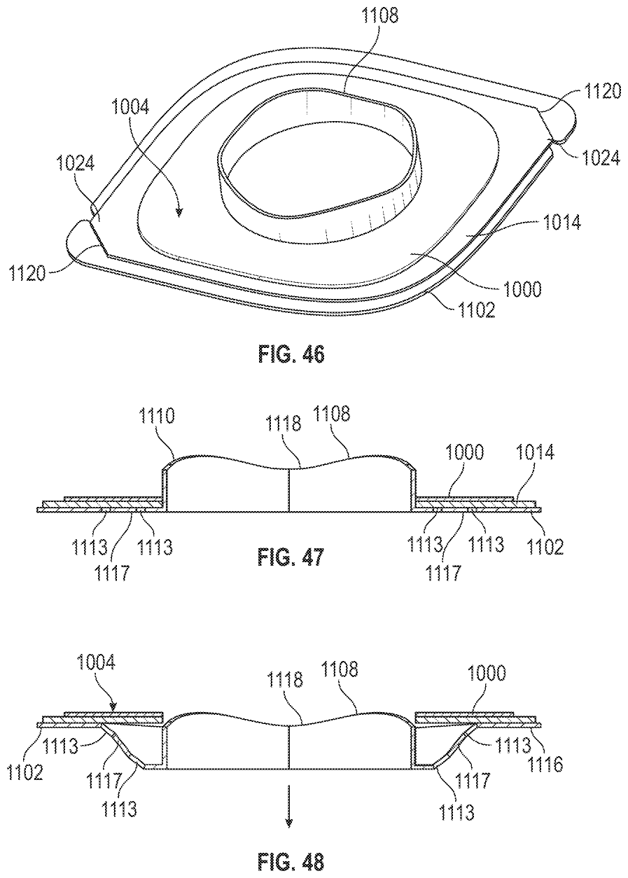

The retainers 1120 may be configured to engage with the retention portions 1024 (marked in FIG. 29) of the backing layer 1014 in examples. The retention portions 1024 may slide into the retainers 1120 in the form of slots to engage with the retainers 1120 (as shown in FIG. 46 for example). Other forms of retention may be utilized in examples.

FIG. 45 illustrates a top view of the applicator 1100.

FIG. 46 illustrates the overlay patch 1000 and the backing layer 1014 positioned upon the substrate 1102. The retention portions 1024 are slid into the retainers 1120. The alignment body 1108 extends through the central openings of the respective overlay patch 1000 and the backing layer 1014. The alignment body 1108 may align the overlay patch 1000 upon the substrate 1102 (e.g., guide the overlay patch 1000 to a defined position upon the substrate 1102). The retainers 1120 may serve to align the overlay patch 1000 upon the substrate 1102.

The overlay patch 1000 may be positioned upon the substrate 1102 with the lower, distal, or engaging surface 1004 facing away from the substrate 1102 and in a distal direction towards the skin of the host. A liner may be utilized and removed prior to application of the overlay patch 1000.

FIGS. 47-48 illustrate an exemplary application procedure of the overlay patch 1000 utilizing the applicator 1100. Features of the on-skin wearable medical device have been excluded from view for clarity. Steps of the application procedure may be varied, excluded, or substituted as desired. Combinations of features of procedures may be used in combination or substitution as desired.

Referring to FIG. 47, a cross sectional view of the overlay patch 1000 and the backing layer 1014 positioned upon the substrate 1102 is shown. A user (such as the host) may position such components upon the substrate 1102. A perspective view of such a configuration is shown in FIG. 46. The substrate 1102 may be in a retracted or proximal position relative to the alignment body 1108.

The on-skin wearable medical device may be disposed upon skin 992 of the host as represented in FIG. 27. The application of the on-skin wearable medical device to the skin 992 may occur through use of an applicator as disclosed herein or via another method as desired.

The alignment body 1108 may be utilized to align the overlay patch 1000 with the on-skin wearable medical device. For example, the on-skin wearable housing 962 may be positioned within the central opening 1110 of the alignment body 1108 to align the overlay patch 1000 with the on-skin wearable medical device. A leading edge 1118 of the alignment body 1108 may be positioned upon the patch 922 or underlay patch surrounding the on-skin wearable housing 962. The alignment body 1108 may guide the applicator 1100 to a desired position. A tactile indication of the position of the alignment body 1108 relative to the on-skin wearable medical device may be provided by a feel of the on-skin wearable housing 962 being inserted into the central opening 1110. Such tactile indication may be beneficial if the on-skin wearable medical device is in a position that is difficult to visualize (e.g., on a back surface of an arm or other position).

Referring to FIG. 48, the substrate 1102 or the outer portion 1116 of the substrate 1102 may be pressed in a direction towards the skin 992 of the host. The alignment body 1108 may be impeded from distal movement due the contact between the leading edge 1118 of the alignment body 1108 and the patch 922 or underlay patch surrounding the on-skin wearable housing 962. The outer portion 1116 of the substrate 1102 accordingly may displace distally relative to the alignment body 1108. The overlay patch 1000 may advance distally towards the on-skin wearable medical device and the skin 992 of the host as represented in FIG. 48.

The displacement portion 1114 of the substrate 1102 may elongate or increase in size axially to allow the outer portion 1116 of the substrate 1102 to displace distally relative to the alignment body 1108. For example, represented in FIG. 48, the size of the one or more voids 1113 may increase, and the displacement portion 1114 may form a conical shape upon displacement of the alignment body 1108 relative to the outer portion 1116 of the substrate 1102. The displacement of the alignment body 1108 may allow the lower, distal, or engaging surface 1004 of the overlay patch 1000 to contact at least a portion of the on-skin wearable medical device and/or the skin 992.

The overlay patch 1000 may be coplanar with the leading edge 1118 of the alignment body 1108 upon application. Other positions may be utilized as desired.

The overlay patch 1000 may have a position as shown in FIG. 31 upon application. The overlay patch 1000 may be positioned upon the patch 922 or underlay patch of the on-skin wearable medical device, or on another portion of the on-skin wearable medical device as desired. A portion of the overlay patch 1000 may be positioned radially outward of the patch 922 or underlay patch for application directly to the skin 992 of the host.

The applicator 1100 may be removed or withdrawn from the skin. The retention portions 1024 of the backing layer 1014 may be decoupled from the one or more retainers 1120. For example, the tabs comprising the retention portions 1024 may be removed from the retainers 1120 comprising slots. The backing layer 1014 may be separated from the overlay patch 1000.

In examples, the overlay patch 1000 may be further pressed or smoothed in position as discussed in regard to FIG. 42.

FIG. 43 may illustrate a resulting configuration of the overlay patch 1000 applied onto the patch 922 or underlay patch of the on-skin wearable medical device, and applied to the skin 992 of the host. A perspective view of the resulting configuration may be shown in FIG. 31.

The use of the applicator may beneficially improve the case upon which the overlay patch may be applied to a desired application site.

Variations in the configuration of the applicator 1100 may be provided.

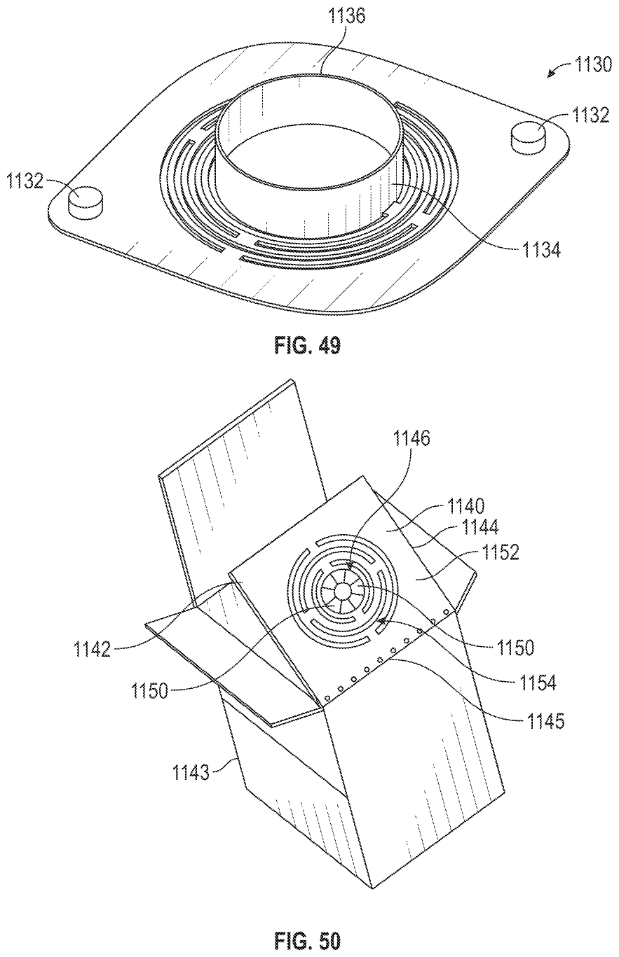

FIG. 49, for example, illustrates a variation in which the applicator 1130 includes retainers 1132 for the overlay patch 1000 in the form of protrusions. The retainers 1132 may be configured to engage a backing layer of the overlay patch 1000 or another component to retain the overlay patch 1000 to the applicator 1130.

The alignment body 1134 of the applicator 1130 may include a leading edge 1136 that has a uniform height (rather than the varied height shown in FIG. 44). The features of the applicator 1100 may otherwise be utilized with the applicator 1130. Features of the applicator 1130 may be utilized in combination or substitution with the features of the applicator 1100.

The features of the examples of FIGS. 44-49 may be utilized solely or in combination with any other example disclosed herein.

Various other configurations of applicators may be utilized in examples.

FIG. 50, for example, illustrates a perspective view of an applicator 1140 for supporting the patch 1000 and configured to apply the patch 1000 onto at least a portion of an on-skin wearable medical device when the on-skin wearable medical device is disposed on a skin of the host.

The applicator 1140 may include a substrate 1142 for supporting the overlay patch 1000. The substrate 1142 may provide a similar function as the substrate 1102 shown in FIG. 44 for example. The substrate 1142, in examples, may comprise a planar surface for the overlay patch 1000 to be positioned upon. The overlay patch 1000 (marked in FIG. 28) may extend parallel with the planar surface upon being positioned upon the planar surface. In examples, other forms of substrates may be utilized.

The substrate 1142 may include an outer periphery 1144 that may have a diameter or width that is at or greater than the diameter or width of the overlay patch 1000 or the backing layer 1014. The substrate 1142 accordingly may support the full diameter or width of the overlay patch 1000 or backing layer 1014 to the respective outer periphery of the overlay patch 1000 or backing layer 1014. Other configurations may be utilized in examples.

The substrate 1142 may include a central portion 1146. The central portion 1146 may correspond to the position of the central portion or overlap portion 1006 (marked in FIG. 28) of the overlay patch 1000 in examples.

The applicator 1140 may comprise a flat sheet of material in examples. The applicator 1140 may be configured to be folded to form features of the applicator 1140 in examples. The folding accordingly may raise portions of the applicator 1140 such that the applicator 1140 is no longer flat. The flattened configuration of the applicator 1140 may be configured to improve shipping and transport of the applicator 1140. For example, a flattened applicator 1140 may be easier to ship or transport than an applicator 1140 having varied heights.

In examples, the applicator 1140 may comprise packaging for another device. For example, the other device may comprise the on-skin wearable medical device or an applicator for the on-skin wearable medical device. In examples, the applicator 1140 may comprise packaging for the overlay patch. Referring to FIG. 50, the applicator 1140 is shown comprising a flap of a box 1143 (i.e., packaging) for the on-skin wearable medical device. The applicator 1140 may be removed at a separation line 1145 or separation portion that allow the applicator 1140 to be removed from the remainder of the packaging. The applicator 1140 may comprise other forms of packaging in examples.

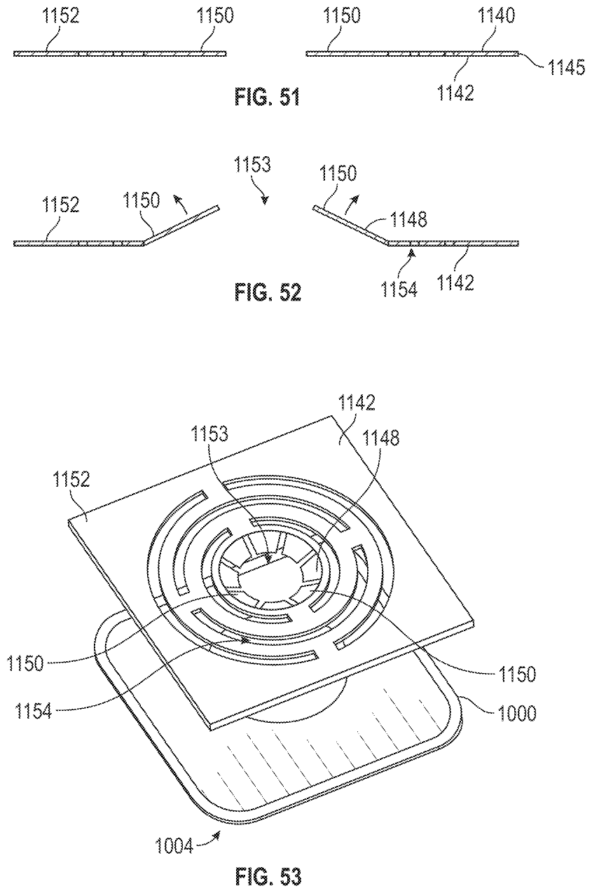

In examples, the applicator 1140 may include an alignment body 1148 (marked in FIG. 52). The alignment body 1148 may be configured to be formed by folding material from a flattened configuration to a raised configuration. For example, FIG. 51 illustrates a side cross sectional view of the applicator 1140 in the flattened configuration. The applicator 1140 may be in the flattened configuration for shipping or transport or for other purposes. The applicator 1140 may be separated from the packaging shown in FIG. 50 for example.

The alignment body 1148 may be formed by folding one or more flaps 1150 relative to an outer portion 1152 of the substrate 1142. The flaps 1150 may be folded to raise the flaps 1150 above a surface of the substrate 1142 to form the alignment body 1148. FIG. 51, for example, illustrates the flaps 1150 in a flattened configuration and FIG. 52 illustrates the flaps 1150 having been folded to a raised configuration.

Referring to FIG. 50, the flaps 1150 may have a variety of configurations and may comprise one or more wedge shaped portions of the substrate 1142. The wedge shaped portions may be oriented to extend radially outward from the center of the substrate 1142. Upon deflection, the flaps 1150 may form a raised cylindrical or conical shape as represented in FIG. 53 for example. Other shapes of folded material may comprise the alignment body 1148 in examples.

The alignment body 1148 may provide a similar function as the alignment body 1108 shown in FIG. 44 for example. The alignment body 1148 may be configured to align the overlay patch 1000 with the on-skin wearable medical device. For example, the alignment body 1148 may orient the applicator 1100 to be positioned at a desired location relative to the on-skin wearable medical device upon application of the overlay patch 1000. The alignment body 1148 may be configured to align with the on-skin wearable housing 962 (marked in FIG. 27) in examples. The alignment body 1148 may center the overlay patch 1000 relative to the on-skin wearable housing 962. The overlay patch 1000 accordingly may be disposed to the on-skin wearable medical device with the central opening 1010 (marked in FIG. 31) of the overlay patch 1000 surrounding the on-skin wearable housing 962. A configuration as shown in FIG. 31 accordingly may result.

The alignment body may extend from the central portion 1146 of the substrate 1102 in examples. Other positions of the alignment body may be utilized in examples.

In examples, the alignment body 1148 may comprise a protrusion formed by the one or more flaps 1150. The protrusion may be configured to extend towards the skin of the host upon the applicator 1140 applying the overlay patch 1000 onto at least a portion of the on-skin wearable medical device. The protrusion, in examples, may include a central opening 1153 that may be configured to receive a portion of the on-skin wearable medical device. The central opening 1153, for example, may receive the on-skin wearable housing 962. One or more walls of the protrusion formed by the one or more flaps 1150 may bound the central opening 1153 and may guide the alignment body 1148 into position around the on-skin wearable housing 962. The protrusion, for example, may comprise a hollow cylinder or cone with the central opening 1153 comprising a channel for receiving a portion of the on-skin wearable medical device (e.g., the on-skin wearable housing 962). The walls of the protrusion may contact or abut the on-skin wearable housing 962 to align the alignment body 1148 relative to the on-skin wearable medical device. The overlay patch 1000 accordingly may be aligned with the on-skin wearable medical device.

In examples, the alignment body 1148 may be configured to displace relative to the substrate 1142. For example, the alignment body 1148 may be configured to retract from the skin of the host upon the applicator 1140 applying the overlay patch 1000 onto at least a portion of the on-skin wearable medical device. The alignment body 1148 may be configured to retract proximally relative to the substrate 1142 upon movement of the substrate 1142 distally towards the skin of the host.

In examples, the applicator 1140 may include a displacement portion 1154 that may allow the alignment body 1148 to displace relative to the substrate 1142. The displacement portion 1154, for example, may comprise a plurality of voids and connectors of the substrate 1142 that may increase the flexibility of the substrate 1142 at the displacement portion 1154. The configuration of the displacement portion 1154 may be the same as discussed in regard to the displacement portion 1114 of the applicator 1100.

The displacement portion 1154 may be at the central portion 1146 of the substrate 1142 in examples. The outer portion 1152 of the substrate 1142 positioned radially outward of the displacement portion 1154 may be solid or may lack voids and connectors in examples. The outer portion 1152 may be relatively rigid compared to the displacement portion 1154.

In examples, the substrate 1142 may be flexible. For example, the substrate 1142 may comprise a flexible polymer or other material that allows the substrate 1142 (including the displacement portion 1154 and the outer portion 1156) to flex as desired. The flexibility may allow for improved contour to a portion of a host's body upon application to an application site.

The substrate 1142 may comprise a disk in examples, with the alignment body 1148 comprising a cylinder or cone extending from the disk.

Features of any other applicator for an overlay patch disclosed herein may be utilized with the applicator 1140.

FIG. 53 illustrates the applicator 1140 approaching an overlay patch 1000 and configured to support the overlay patch 1000 upon the substrate 1142. The overlay patch 1000 may be positioned upon the substrate 1142 with the lower, distal, or engaging surface 1004 of the overlay patch 1000 facing away from the substrate 1142 and in a distal direction towards the skin of the host. A liner may be utilized and removed prior to application of the overlay patch 1000.

The overlay patch 1000 may be applied in a similar manner as discussed regarding the applicator 1100.

The overlay patch 1000 may have a position as shown in FIG. 31 upon application. The overlay patch 1000 may be positioned upon the patch 922 or underlay patch of the on-skin wearable medical device, or on another portion of the on-skin wearable medical device as desired. A portion of the overlay patch 1000 may be positioned radially outward of the patch 922 or underlay patch for application directly to the skin 992 of the host.

The applicator 1140 may be removed or withdrawn from the skin. In examples, the overlay patch 1000 may be further pressed or smoothed in position as discussed in regard to FIG. 42.

FIG. 43 may illustrate a resulting configuration of the overlay patch 1000 applied onto the patch 922 or underlay patch of the on-skin wearable medical device, and applied to the skin 992 of the host. A perspective view of the resulting configuration may be shown in FIG. 31.

The use of the applicator may beneficially improve the case upon which the overlay patch may be applied to a desired application site.

The features of the examples of FIGS. 50-53 may be utilized solely or in combination with any other example disclosed herein.

Various other configurations of applicators may be utilized in examples.

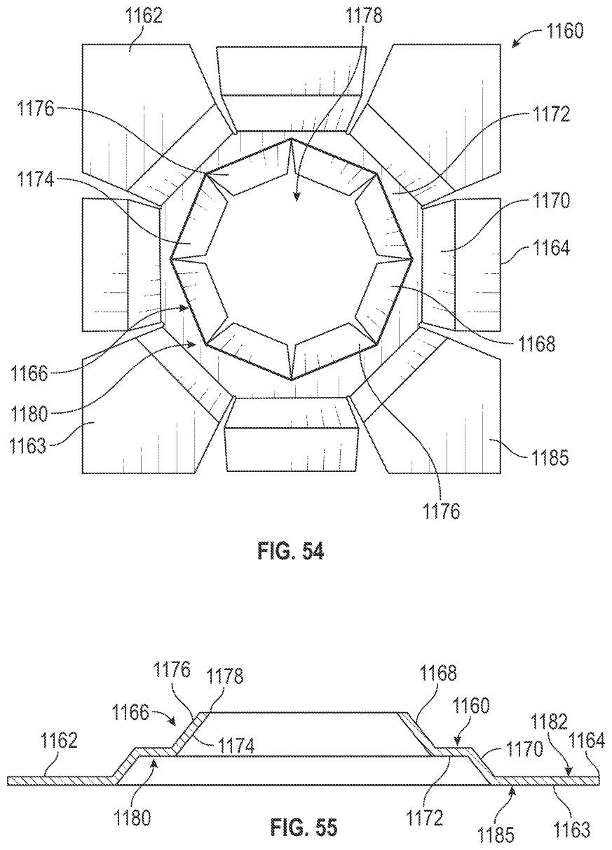

FIGS. 54 and 55, for example, illustrates a perspective view of an applicator 1160 for supporting the patch 1000 and configured to apply the patch 1000 onto at least a portion of an on-skin wearable medical device when the on-skin wearable medical device is disposed on a skin of the host.

FIG. 54 illustrates a proximal view of the applicator 1160. FIG. 55 illustrates a side cross sectional view of the applicator 1160 in the configuration shown in FIG. 54.

The applicator 1160 may include a substrate 1162 for supporting the overlay patch 1000. The substrate 1162 may provide a similar function as the substrate 1102 shown in FIG. 44 for example. The substrate 1162, in examples, may comprise a planar surface for the overlay patch 1000 to be positioned upon. The overlay patch 1000 may extend parallel with the planar surface upon being positioned upon the planar surface. In examples, other forms of substrates may be utilized.

The substrate 1162 may include an outer periphery 1164 that may have a diameter or width that is at or greater than the diameter or width of the overlay patch 1000 or the backing layer 1014. The substrate 1162 accordingly may support the full diameter or width of the overlay patch 1000 or backing layer 1014 to the respective outer periphery of the overlay patch 1000 or backing layer 1014. Other configurations may be utilized in examples.

The substrate 1162 may include a central portion 1166. The central portion 1166 may correspond to the position of the central portion or overlap portion 1006 (marked in FIG. 28) of the overlay patch 1000 in examples.

The applicator 1160 may comprise a flat sheet of material in examples. The applicator 1160 may be configured to be folded to form features of the applicator 1160 in examples. The folding accordingly may raise portions of the applicator 1160 such that the applicator 1160 is no longer flat. The flattened configuration of the applicator 1160 may be configured to improve shipping and transport of the applicator 1160 in a similar manner as discussed regarding the applicator 1140.

In examples, the applicator 1160 may comprise packaging for another device, in a similar manner as discussed regarding the applicator 1140.

In examples, the applicator 1160 may include an alignment body 1168. The alignment body 1168 may be configured to be formed by folding material from a flattened configuration to a raised configuration. The flattened configuration may comprise a flattened sheet of material as represented in FIG. 51 for example. FIGS. 54 and 55 illustrate the alignment body 1168 already folded into the raised configuration. The alignment body 1168 may be folded to form steps, levels, or plateaus that raise the alignment body 1168. For example, a first level 1170 may comprise a raised portion that raises the alignment body 1168 from the substrate 1162. The first level 1170 may be angled as desired. A second level 1172 may comprise a plateau portion that extend radially inward from the first level 1170. A third level 1174 may comprise a raised portion that raises the alignment body 1168 from the second level 1172. The third level 1174 may be angled as desired. The alignment body 1168 may be folded into the configuration shown in FIGS. 54 and 55 from a flattened configuration.

The alignment body 1168 may be formed by folding one or more flaps 1176, in a similar manner as discussed regarding the flaps 1150 of the applicator 1140. Upon deflection, the alignment body 1168 may form a raised cylindrical or conical shape as represented in FIGS. 54 and 55 for example. Other shapes of folded material may comprise the alignment body 1168 in examples.

The alignment body 1168 may provide a similar function as the alignment body 1108 shown in FIG. 44 for example. The alignment body 1168 may be configured to align the overlay patch 1000 with the on-skin wearable medical device. For example, the alignment body 1168 may orient the applicator 1160 to be positioned at a desired location relative to the on-skin wearable medical device upon application of the overlay patch 1000. The alignment body 1168 may be configured to align with the on-skin wearable housing 962 (marked in FIG. 27) in examples. The alignment body 1168 may center the overlay patch 1000 relative to the on-skin wearable housing 962. The overlay patch 1000 accordingly may be disposed to the on-skin wearable medical device with the central opening 1010 (marked in FIG. 31) of the overlay patch 1000 surrounding the on-skin wearable housing 962. A configuration as shown in FIG. 31 accordingly may result.

The alignment body 1168 may extend from the central portion 1166 of the substrate 1162 in examples. Other positions of the alignment body may be utilized in examples.

In examples, the alignment body 1168 may comprise a protrusion formed by the one or more flaps 1176. The protrusion may be configured to extend towards the skin of the host upon the applicator 1160 applying the overlay patch 1000 onto at least a portion of the on-skin wearable medical device. The protrusion, in examples, may include a central opening 1178 that may be configured to receive a portion of the on-skin wearable medical device. The central opening 1178, for example, may receive the on-skin wearable housing 962. One or more walls of the protrusion formed by the one or more flaps 1176 may bound the central opening 1178 and may guide the alignment body 1168 into position around the on-skin wearable housing 962. The protrusion, for example, may comprise a hollow cylinder or cone with the central opening 1178 comprising a channel for receiving a portion of the on-skin wearable medical device (e.g., the on-skin wearable housing 962). The walls of the protrusion may contact or abut the on-skin wearable housing 962 to align the alignment body 1168 relative to the on-skin wearable medical device. The overlay patch 1000 accordingly may be aligned with the on-skin wearable medical device.

In examples, the alignment body 1168 may be configured to displace relative to the substrate 1162. For example, the alignment body 1168 may be configured to retract from the skin of the host upon the applicator 1160 applying the overlay patch 1000 onto at least a portion of the on-skin wearable medical device. The alignment body 1168 may be configured to retract proximally relative to the substrate 1162 upon movement of the substrate 1162 distally towards the skin of the host.

In examples, the applicator 1160 may include a displacement portion 1180 that may allow a first portion of the applicator 1160 (e.g., the alignment body 1168) to displace relative to a second portion of the applicator 1160 (e.g., the substrate 1162). The displacement portion 1180, for example, may comprise the steps, levels, or plateaus that raise the alignment body 1168. The displacement portion 1180 may be configured to collapse or invert upon application of the overlay patch 1000.

The displacement portion 1180 may be at the central portion 1166 of the substrate 1162 in examples. The outer portion 1163 of the substrate 1162 positioned radially outward of the displacement portion 1180 may be flat or non-collapsible in examples. The outer portion 1163 may be relatively rigid compared to the displacement portion 1180.

In examples, the substrate 1162 may be flexible. For example, the substrate 1162 may comprise a flexible polymer or other material that allows the substrate 1162 (including the displacement portion 1180 and the outer portion 1163) to flex as desired. The flexibility may allow for improved contour to a portion of a host's body upon application to an application site.

The substrate 1162 may comprise a disk in examples, with the alignment body 1168 comprising a cylinder or cone extending from the disk.

Features of any other applicator for an overlay patch disclosed herein may be utilized with the applicators 1140, 1160.

In application, the overlay patch 1000 may be positioned upon a distal surface 1182 (marked in FIG. 55) of the substrate 1162 that faces opposite a proximal surface 1185 (marked in FIGS. 54 and 55) of the substrate 1162. The lower, distal, or engaging surface 1004 of the overlay patch 1000 may face away from the substrate 1162 and in a distal direction towards the skin of the host. A liner may be utilized and removed prior to application of the overlay patch 1000.

The overlay patch 1000 may be applied in a similar manner as discussed regarding the applicator 1100.

The overlay patch 1000 may have a position as shown in FIG. 31 upon application. The overlay patch 1000 may be positioned upon the patch 922 or underlay patch of the on-skin wearable medical device, or on another portion of the on-skin wearable medical device as desired. A portion of the overlay patch 1000 may be positioned radially outward of the patch 922 or underlay patch for application directly to the skin 992 of the host.

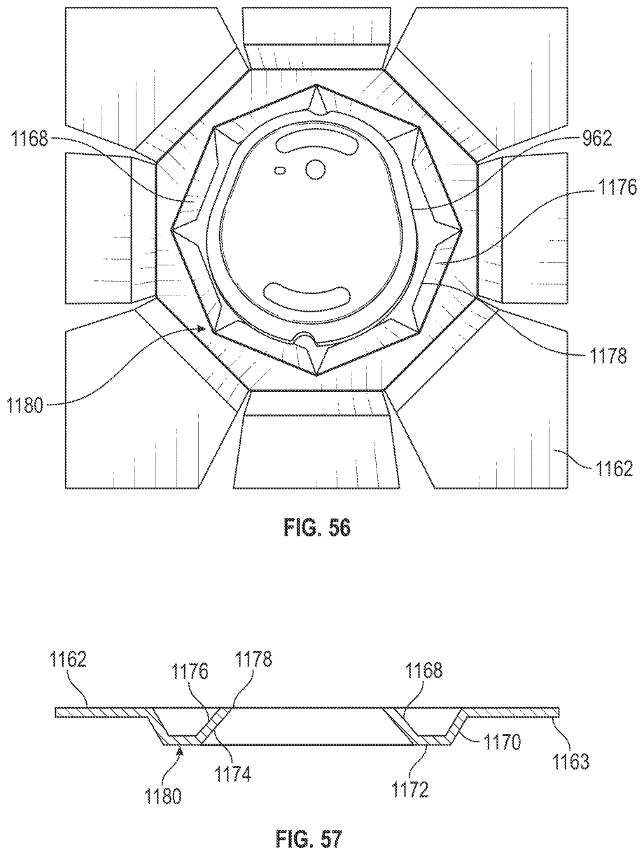

FIGS. 56 and 57 illustrate a resulting configuration of the applicator 1160 upon application of the overlay patch 1000. The on-skin wearable housing 962 is shown within the central opening 1178 in FIG. 56. The on-skin wearable housing 962 is excluded from view from FIG. 57 for clarity.

In FIGS. 56 and 57, the displacement portion 1180 has collapsed or inverted upon application of the overlay patch 1000. For example, the angle of the first level 1170 has inverted such that the first level 1170 extends proximally in FIG. 57 (rather than distally as shown in FIG. 55). The second level 1172 remains a plateau portion, yet positioned proximal of the outer portion 1163 of the substrate 1162 (rather than distal of the outer portion 1163 as shown in FIG. 55). The third level 1174 is angled in a distal direction as shown in FIG. 55, yet is positioned proximal of the position shown in FIG. 55. The displacement portion 1180 has deflected to allow the alignment body 1168 to displace proximally relative to the substrate 1162.

The applicator 1160 may be removed or withdrawn from the skin. In examples, the overlay patch 1000 may be further pressed or smoothed in position as discussed in regard to FIG. 42.

FIG. 43 may illustrate a resulting configuration of the overlay patch 1000 applied onto the patch 922 or underlay patch of the on-skin wearable medical device, and applied to the skin 992 of the host. A perspective view of the resulting configuration may be shown in FIG. 31.

The use of the applicator may beneficially improve the case upon which the overlay patch may be applied to a desired application site.

The features of the examples of FIGS. 54-57 may be utilized solely or in combination with any other example disclosed herein.

Various other configurations of applicators may be utilized in examples.

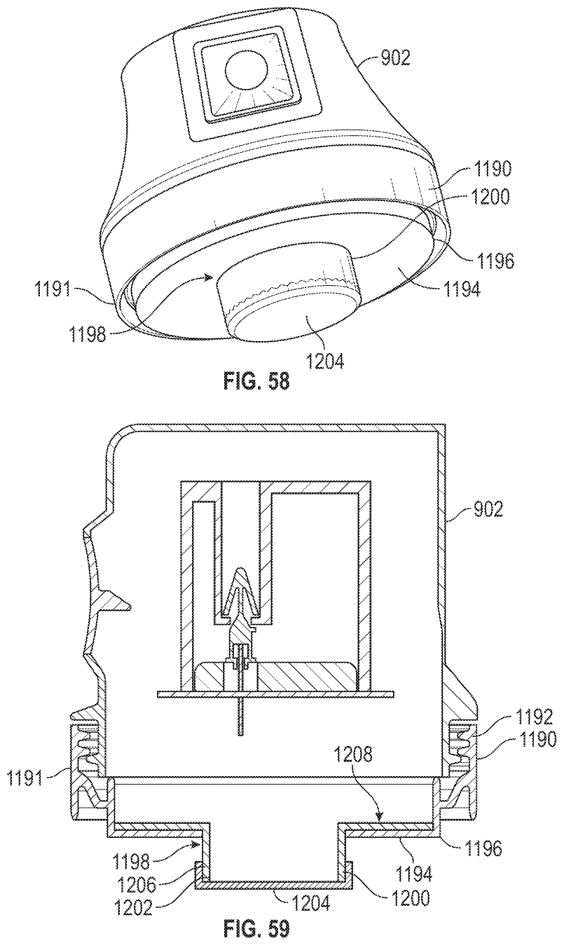

FIG. 58, for example, illustrates a perspective view of an applicator 1190 for supporting the patch 1000 and configured to apply the patch 1000 onto at least a portion of an on-skin wearable medical device when the on-skin wearable medical device is disposed on a skin of the host.

The applicator 1190 may comprise a cap 1191 for an applicator housing of an on-skin wearable medical device in examples. The applicator housing may be configured similarly as the applicator housing 902 shown in FIG. 25 or may have another configuration in examples. FIG. 59, for example, illustrates a cross sectional view of the applicator housing 902 with the cap 1191 utilized in substitution of the cap 942 shown in FIG. 25. The cap 1191 may include features similar to the cap 942 shown in FIG. 25, for example, an engagement portion 1192 or threading for coupling to the applicator housing 902. The cap 1191 may cover a distal opening of the applicator housing 902 in a similar manner as the cap 942. Features of the applicator 900 shown in FIG. 25 are excluded from view for clarity in FIG. 59, yet may be included within the applicator housing 902 as desired.

Referring to FIG. 58, the applicator 1190 for the overlay patch 1000 may include a substrate 1194 for supporting the overlay patch 1000. The substrate 1194 may provide a similar function as the substrate 1052 shown in FIG. 34 for example. The substrate 1194, in examples, may comprise a planar surface for the overlay patch 1000 to be positioned upon. The overlay patch 1000 may extend parallel with the planar surface upon being positioned upon the planar surface. In examples, other forms of substrates may be utilized.

The substrate 1194 may include an outer periphery 1196 that may have a diameter or width that is at or greater than the diameter or width of the overlay patch 1000 or the backing layer 1014. The substrate 1194 accordingly may support the full diameter or width of the overlay patch 1000 or backing layer 1014 to the respective outer periphery of the overlay patch 1000 or backing layer 1014. Other configurations may be utilized in examples.

The substrate 1194 may include a central portion 1198. The central portion 1198 may correspond to the position of the central portion or overlap portion 1006 (marked in FIG. 28) of the overlay patch 1000 in examples.

In examples, one or more retainers may be utilized to retain the overlay patch 1000 to the substrate 1194. For example, retainers 1132 as shown in FIG. 49 or any other form of retainer may be utilized as desired.

In examples, the applicator 1190 may include an alignment body 1200. The alignment body 1200 may provide a similar function as the alignment body 1058 shown in FIG. 34 for example. The alignment body 1200 may be configured to align the overlay patch 1000 with the on-skin wearable medical device. For example, the alignment body 1200 may orient the applicator 1190 to be positioned at a desired location relative to the on-skin wearable medical device upon application of the overlay patch 1000. The alignment body 1200 may be configured to align with the on-skin wearable housing 962 (marked in FIG. 27) in examples. The alignment body 1200 may center the overlay patch 1000 relative to the on-skin wearable housing 962. The overlay patch 1000 accordingly may be disposed to the on-skin wearable medical device with the central opening 1010 (marked in FIG. 31) of the overlay patch 1000 surrounding the on-skin wearable housing 962. A configuration as shown in FIG. 31 accordingly may result.

The alignment body 1200 may extend from the central portion 1 198 of the substrate 1194 in examples. Other positions of the alignment body may be utilized in examples.

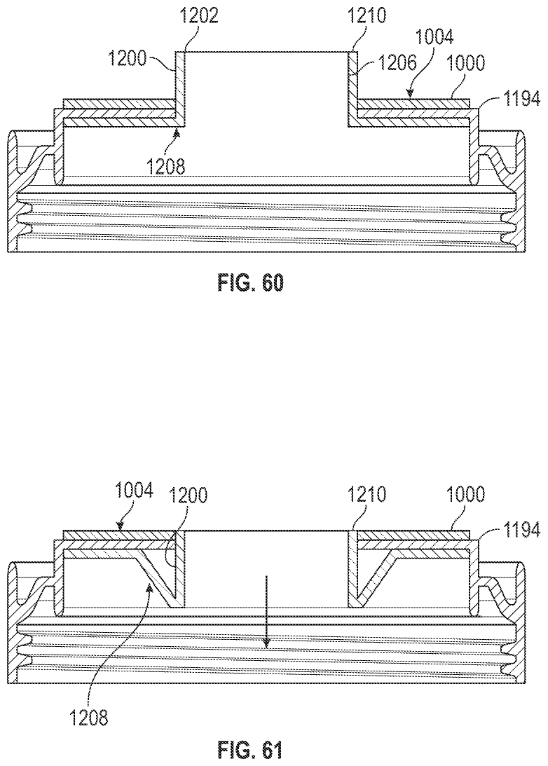

In examples, the alignment body 1200 may comprise a protrusion. The protrusion may be configured to extend towards the skin of the host upon the applicator 1190 applying the overlay patch 1000 onto at least a portion of the on-skin wearable medical device. The protrusion, in examples, may include a central opening 1202 (marked in FIGS. 59 and 60) that may be configured to receive a portion of the on-skin wearable medical device. The central opening 1202, for example, may receive the on-skin wearable housing 962. The protrusion may comprise a cylinder extending from the substrate 1194.

Referring to FIG. 58, in examples, the central opening 1202 may be covered by a barrier layer 1204. The barrier layer 1204 may provide a similar function as other barrier layers for an applicator housing disclosed herein. The barrier layer 1204, for example, may seal an interior of the applicator housing 902. The barrier layer 1204 may allow for transmission of sterilizing gas (e.g., ethylene oxide (EO) gas) therethrough to sterilize the interior of the applicator housing 902. The barrier layer 1204 may be permeable to sterilizing gas to allow for sterilization. The barrier layer 1204 may comprise a lid upon the central opening 1202 in examples. The barrier layer 1204 may be removed prior to use of the applicator 1190, and preferably after the applicator 1190 or cap 1191 has been removed from the applicator housing 902. The barrier layer 1204, for example, may be peeled off by a user (such as a host) prior to use of the applicator 1190. The barrier layer 1204 having been removed is represented in FIG. 60.

One or more walls 1206 of the protrusion may bound the central opening 1202 and may guide the alignment body 1200 into position around the on-skin wearable housing 962. The protrusion, for example, may comprise a hollow cylinder with the central opening 1202 comprising a channel for receiving a portion of the on-skin wearable medical device (e.g., the on-skin wearable housing 962). The channel may allow the sterilizing gas to pass through to contact components interior of the housing 902. The walls 1206 of the protrusion may contact or abut the on-skin wearable housing 962 to align the alignment body 1200 relative to the on-skin wearable medical device. The overlay patch 1000 accordingly may be aligned with the on-skin wearable medical device.

In examples, the alignment body 1200 may be configured to displace relative to the substrate 1194. For example, the alignment body 1200 may be configured to retract from the skin of the host upon the applicator 1190 applying the overlay patch 1000 onto at least a portion of the on-skin wearable medical device. The alignment body 1200 may be configured to retract proximally relative to the substrate 1194 upon movement of the substrate 1194 distally towards the skin of the host.

In examples, the applicator 1190 may include a displacement portion 1208 that may allow a first portion of the applicator 1190 (e.g., the alignment body 1200) to displace relative to a second portion of the applicator 1190 (e.g., the substrate 1194). The displacement portion 1208, for example, may comprise a flexible portion or deflectable portion of the applicator 1190 that may bend or otherwise deflect to allow the alignment body 1200 to displace relative to the substrate 1194. The displacement portion 1208 may be more flexible than the substrate 1194.

The displacement portion 1208 may be at the central portion 1198 of the substrate 1194 in examples. Referring to FIGS. 60 and 61, the displacement portion 1208 may comprise a layer of material positioned proximal of the substrate 1194 and configured to deflect or bend proximally.

In examples, the alignment body 1200 may have a leading edge 1210 that is configured to face towards the skin of the host upon the applicator 1190 applying the overlay patch 1000 onto a portion of the on-skin wearable medical device. The leading edge 1210 may have a uniform height or a varied height in examples.

The applicator 1190 may be removed from the applicator housing 902 prior to use of the applicator 1190. In examples, the applicator 1190 may be utilized to apply the overlay patch 1000 to an application site while remaining coupled to the applicator housing 902. In an example in which the applicator 1190 is removed from the applicator housing 902, the applicator 1190 may be unscrewed or otherwise decoupled from the applicator housing 902. The barrier layer 1204 may be removed.

Referring to FIG. 60, the overlay patch 1000 may be positioned upon the substrate 1194. A backing layer 1014 may further be positioned upon the substrate as desired, or may be removed prior to use. FIG. 60 illustrates the overlay patch 1000 positioned upon the substrate 1194. The alignment body 1200 extends through the central opening of the overlay patch 1000. The alignment body 1200 may align the overlay patch 1000 upon the substrate 1194 (e.g., guide the overlay patch 1000 to a defined position upon the substrate 1194).

The overlay patch 1000 may be positioned upon the substrate 1194 with the lower, distal, or engaging surface 1004 facing away from the substrate 1194 and in a distal direction towards the skin of the host. A liner may be utilized and removed prior to application of the overlay patch 1000.

FIGS. 60 and 61 represent an exemplary application procedure of the overlay patch 1000 utilizing the applicator 1190. Features of the on-skin wearable medical device have been excluded from view for clarity. Steps of the application procedure may be varied, excluded, or substituted as desired. Combinations of features of procedures may be used in combination or substitution as desired.

Referring to FIG. 60, a cross sectional view of the overlay patch 1000 positioned upon the substrate 1194 is shown. A user (such as the host) may position such components upon the substrate 1194. The substrate 1194 may be in a retracted or proximal position relative to the alignment body 1200.

The on-skin wearable medical device may be disposed upon skin 992 of the host as represented in FIG. 27. The application of the on-skin wearable medical device to the skin 992 may occur through use of an applicator as disclosed herein or via another method as desired.

The alignment body 1200 may be utilized to align the overlay patch 1000 with the on-skin wearable medical device. For example, the on-skin wearable housing 962 may be positioned within the central opening 1202 of the alignment body 1200 to align the overlay patch 1000 with the on-skin wearable medical device. The leading edge 1210 of the alignment body 1200 may be positioned upon the patch 922 or underlay patch surrounding the on-skin wearable housing 962. The alignment body 1200 may guide the applicator 1190 to a desired position. A tactile indication of the position of the alignment body 1200 relative to the on-skin wearable medical device may be provided by a feel of the on-skin wearable housing 962 being inserted into the central opening 1202. Such tactile indication may be beneficial if the on-skin wearable medical device is in a position that is difficult to visualize (e.g., on a back surface of an arm or other position).

Referring to FIG. 61, the substrate 1194 may be pressed in a direction towards the skin 992 of the host. The alignment body 1200 may be impeded from distal movement due the contact between the leading edge 1210 of the alignment body 1200 and the patch 922 or underlay patch surrounding the on-skin wearable housing 962. The substrate 1194 accordingly may displace distally relative to the alignment body 1200 as represented in FIG. 61. The overlay patch 1000 may advance distally towards the on-skin wearable medical device and the skin 992 of the host.

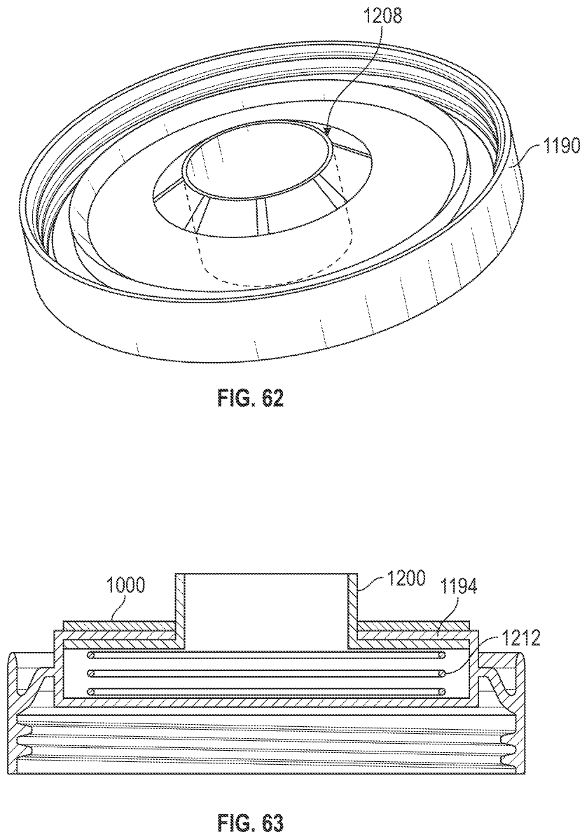

The displacement portion 1208 of the applicator 1190 may deflect axially to allow the substrate 1194 to displace distally relative to the alignment body 1200. FIG. 62, for example, illustrates a perspective view of the deflection of the displacement portion 1208. The displacement of the alignment body 1200 may allow the lower, distal, or engaging surface 1004 of the overlay patch 1000 to contact at least a portion of the on-skin wearable medical device and/or the skin 992.

The overlay patch 1000 may be coplanar with the leading edge 1210 of the alignment body 1108 upon application. Other positions may be utilized as desired.

The overlay patch 1000 may have a position as shown in FIG. 31 upon application. The overlay patch 1000 may be positioned upon the patch 922 or underlay patch of the on-skin wearable medical device, or on another portion of the on-skin wearable medical device as desired.

A portion of the overlay patch 1000 may be positioned radially outward of the patch 922 or underlay patch for application directly to the skin 992 of the host.

The applicator 1190 may be removed or withdrawn from the skin. In examples, the overlay patch 1000 may be further pressed or smoothed in position as discussed in regard to FIG. 42.

FIG. 43 may illustrate a resulting configuration of the overlay patch 1000 applied onto the patch 922 or underlay patch of the on-skin wearable medical device, and applied to the skin 992 of the host. A perspective view of the resulting configuration may be shown in FIG. 31.

The use of the applicator may beneficially improve the case upon which the overlay patch may be applied to a desired application site.

Variations in the configuration of the applicator 1190 may be provided.

FIG. 63, for example, illustrates a variation in which a spring 1212 may be utilized to bias the alignment body 1200 relative to the substrate 1194. The spring 1212 may bias the alignment body 1200 to be raised relative to the substrate 1194. The spring 1212 may comprise a coil spring or other form of spring. The bias of the spring 1212 may be overcome to allow the alignment body 1200 to displace relative to the substrate 1194 in an application process.

The features of the examples of FIGS. 58-63 may be utilized solely or in combination with any other example disclosed herein.

Various other configurations of applicators may be utilized in examples.

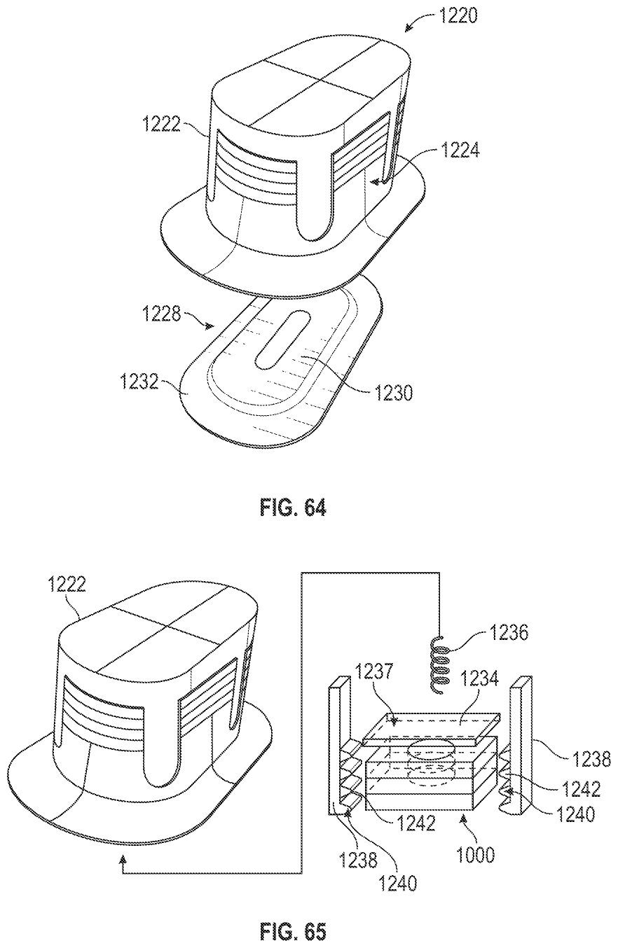

FIGS. 64-68 illustrate a configuration of an applicator 1220 for supporting a patch 1000 and configured to apply the patch 1000 onto at least a portion of an on-skin wearable medical device when the on-skin wearable medical device is disposed on a skin of a host. The applicator 1220 may be configured to apply multiple overlay patches in examples. In examples, the applicator 1220 may be configured to apply a first overlay patch to at least a portion of an on-skin wearable medical device and automatically position a second overlay patch for application to at least a portion of an on-skin wearable medical device upon application of the first overlay patch.

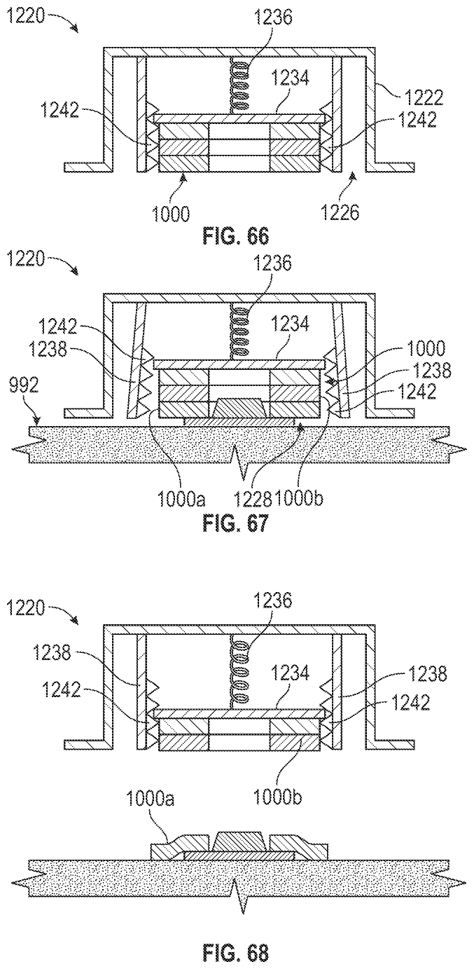

FIG. 64, for example, illustrates a perspective view of the applicator 1220. The applicator 1220 may include a housing 1222 that may contain components of the applicator 1220 in examples. The housing 1222 may include an outer surface 1224 for grip by a user (such as a host) in examples. Referring to the cross sectional view of FIG. 66, the housing 1222 may include an opening 1226, such as a distal opening, for one or more overlay patches to be deployed or dispensed from.

The applicator 1220 is shown in a position to approach an on-skin wearable medical device 1228 disposed upon skin of a host in FIG. 64. The on-skin wearable medical device 1228 may comprise any form of on-skin wearable medical device disclosed herein, and may include an on-skin wearable housing 1230 and a patch 1232 or underlay patch in examples.

FIG. 65 illustrates a view of components of the applicator 1220 in examples. The applicator 1220 may include a substrate 1234 for supporting one or more of the overlay patches 1000 in examples. The overlay patches 1000 may be stacked upon each other in a column arrangement, although other configurations may be utilized. The substrate 1234 may be configured to apply a distal force against the plurality of overlay patches 1000. The substrate 1234 may comprise a planar surface for the overlay patch 1000 to be positioned upon and extend parallel with the planar surface. A spring 1236, or other component, for example, may exert a distal force against a proximal surface 1237 of the substrate 1234 to press the overlay patches 1000 in a distal direction.

In examples, the applicator 1220 may include one or more retainers 1238 configured to retain the overlay patches 1000 to the applicator 1220. The configuration of the retainers 1238 may vary in examples. As shown in FIG. 65, the retainers 1238 may comprise arms having engagement portions 1240 for engaging the overlay patches 1000. The engagement portions 1240 may comprise protrusions 1242 (e.g., teeth or serrations) that may engage the overlay patches 1000 such as side surfaces of the overlay patches 1000. The protrusions 1242 may be angled to allow for deflection by the substrate 1234 as disclosed herein. The applicator 1220 may be configured to retain a plurality of overlay patches 1000 in examples.

FIG. 66 illustrates a side cross sectional view of the applicator 1220 retaining a plurality of the overlay patches 1000. The protrusions 1242, for example, may retain the overlay patches 1000 and retain the substrate 1234 in position. The protrusions 1242 may resist the distal force applied by the spring 1236 to the substrate 1234 and accordingly the overlay patches 1000. The applicator 1220 may approach an on-skin wearable medical device 1228 disposed upon skin 992 of a host as represented in FIG. 67.

FIG. 67 illustrates the applicator 1220 applying one of the overlay patches 1000 to at least a portion of the on-skin wearable medical device 1228. The overlay patches 1000 may include a first overlay patch 1000a and a second overlay patch 1000b in examples. The applicator 1220 may be configured to apply the first overlay patch 1000a to at least a portion of the on-skin wearable medical device 1228 and automatically position the second overlay patch 1000b for application to at least a portion of an on-skin wearable medical device, which may be the same on-skin wearable medical device 1228 or a different on-skin wearable medical device. The applicator 1220 may be configured to apply the second overlay patch 1000b at a later time to the same on-skin wearable medical device 1228 if desired.

The applicator 1220 may operate to release the first overlay patch 1000a for application to the on-skin wearable medical device and to retain the second overlay patch 1000b. A variety of deployment mechanisms may be utilized. For example, the configuration shown in FIG. 67 includes the on-skin wearable medical device pressing proximally against the plurality of overlay patches 1000 or the stack of overlay patches 1000. The proximal force against the plurality of overlay patches 1000 may compress the spring 1236. The substrate 1234 may move proximally due to the compression of the spring 1236. The proximal movement of the substrate 1234 may press outward against the protrusions 1242. For example, as shown in FIG. 67, the substrate 1234 may have a greater width or diameter than the overlay patches 1000 thus causing the outer periphery of the substrate 1234 to press proximally against the protrusions 1242. The protrusion 1242 may be angled such that such proximal movement causes the retainers 1238 to deflect outwardly as shown in FIG. 67. The outward deflection of the retainers 1238 may cause the protrusion 1242 to disengage from the overlay patch 1000a and release the overlay patch 1000a for application to the on-skin wearable medical device 1228.

Referring to FIG. 68, the applicator 1220 may be withdrawn, and the proximal force upon the spring 1236 may be reduced. The force of the substrate 1234 upon the protrusions 1242 may be reduced and the retainers 1238 may deflect inward to engage the second overlay patch 1000b. The second overlay patch 1000b may be positioned for application to an on-skin wearable medical device. The applicator 1220 may be iteratively utilized to apply each overlay patch retained by the applicator 1220.

The features of the examples of FIGS. 64-68 may be utilized solely or in combination with any other example disclosed herein.

Various other configurations of applicators may be utilized in examples.

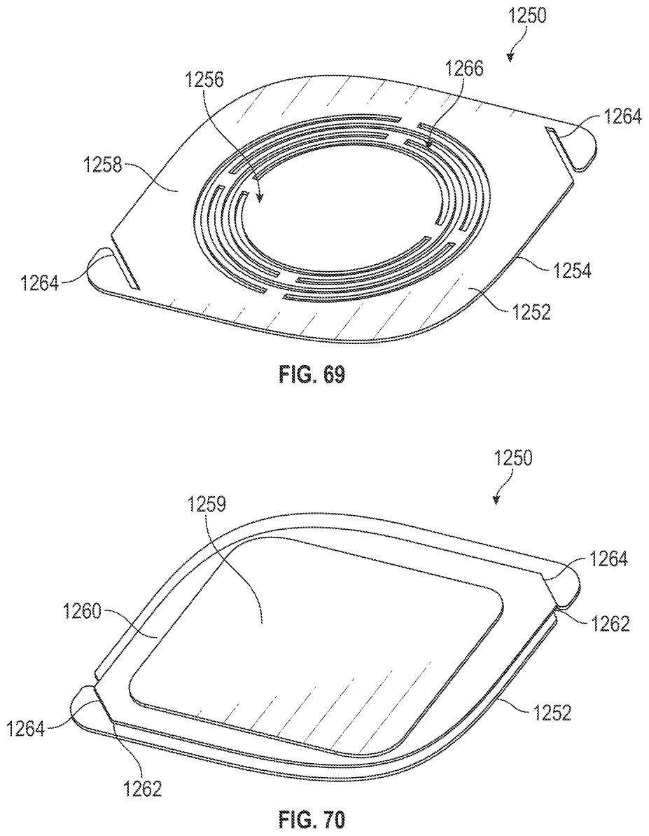

FIG. 69, for example, illustrates a perspective view of an applicator 1250 for supporting an overlay patch and configured to apply an overlay patch onto at least a portion of an on-skin wearable medical device when the on-skin wearable medical device is disposed on a skin of the host.

The applicator 1250 may include a substrate 1252 for supporting an overlay patch. The substrate 1252 may provide a similar function as the substrate 1052 shown in FIG. 34 for example. The substrate 1252, in examples, may comprise a planar surface for an overlay patch to be positioned upon. The overlay patch may extend parallel with the planar surface upon being positioned upon the planar surface. In examples, other forms of substrates may be utilized.

The substrate 1252 may include an outer periphery 1254 that may have a diameter or width that is at or greater than the diameter or width of an overlay patch or a backing layer for the overlay patch. The substrate 1252 accordingly may support the full diameter or width of the overlay patch or backing layer to the respective outer periphery of the overlay patch or backing layer. Other configurations may be utilized in examples.

The substrate 1252 may include a central portion 1256. The central portion 1256 may correspond to the position of a central portion or an overlap portion of an overlay patch in examples. The applicator 1250 may lack an alignment body that is configured similarly as the alignment body 1108 as shown in FIG. 44 for example. The central portion 1256 accordingly may be flat or smooth in examples and may be at a same height as an outer portion 1258 of the substrate 1252. An overlay patch 1259 (marked in FIG. 70) that may be utilized accordingly may lack a central opening. The features of the overlay patch 1259 may otherwise comprise the features of the overlay patch 1000. The overlay patch 1259 may be positioned upon a backing layer 1260 that may be configured similarly as the backing layer 1014 yet may lack a central opening. The backing layer 1260, for example, may have retention portions 1262 that couple to retainers 1264 of the applicator 1250. The retainers 1264 may comprise slots or may have another configuration, similar to the retainers 1120 of the applicator 1100.

In examples, the central portion 1256 may be configured to displace relative to the substrate 1252. For example, the central portion 1256 may be configured to retract from the skin of the host upon the applicator 1250 applying the overlay patch 1259 onto at least a portion of the on-skin wearable medical device. The central portion 1256 may be configured to retract proximally relative to the substrate 1252 upon movement of the substrate 1252 distally towards the skin of the host.

In examples, the applicator 1250 may include a displacement portion 1266 that may allow a first portion of the applicator 1250 (e.g., the central portion 1256) to displace relative to a second portion of the applicator 1250 (e.g., the substrate 1252). The displacement portion 1266, for example, may have a configuration as disclosed in regard to the displacement portion 1114 of the applicator 1100, or may have another configuration as desired.

In examples, the substrate 1252 may be flexible. For example, the substrate 1252 may comprise a flexible polymer or other material that allows the substrate 1252 (including the displacement portion 1266 and the outer portion 1258) to flex as desired. The flexibility may allow for improved contour to a portion of a host's body upon application to an application site.

The substrate 1252 may comprise a disk in examples.

FIG. 70 illustrates a perspective view of the applicator 1250 with the overlay patch 1259 positioned upon the substrate 1252. The retainers 1264 may serve to align the overlay patch 1259 upon the substrate 1252. FIG. 71 illustrates a side cross sectional view of the overlay patch 1259 positioned upon the substrate 1252. The overlay patch 1259 may be positioned upon the substrate 1252 with a lower, distal, or engaging surface of the overlay patch 1259 facing away from the substrate 1252 and in a distal direction towards the skin of the host. A liner may be utilized and removed prior to application of the overlay patch 1259.

The displacement portion 1266 may displace or deflect to allow the applicator 1250 to apply the overlay patch 1259.

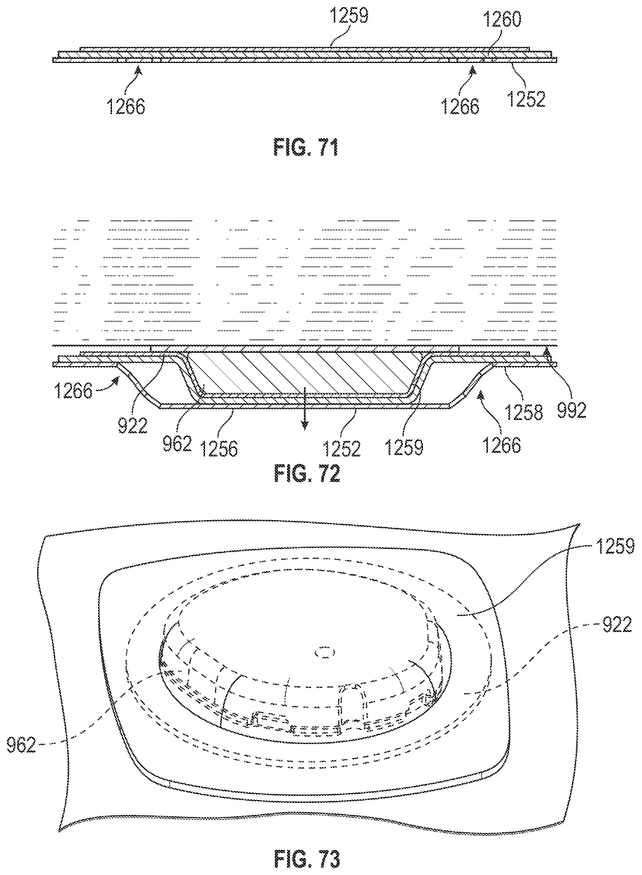

FIG. 72 illustrates an exemplary application procedure of the overlay patch 1259 utilizing the applicator 1250. Steps of the application procedure may be varied, excluded, or substituted as desired. Combinations of features of procedures may be used in combination or substitution as desired.

Referring to FIG. 72, an on-skin wearable medical device may be disposed upon skin 992 of the host as represented in FIG. 27. The application of the on-skin wearable medical device to the skin 992 may occur through use of an applicator as disclosed herein or via another method as desired.

The substrate 1252 or the outer portion 1258 of the substrate 1252 may be pressed in a direction towards the skin 992 of the host. The central portion 1256 may be impeded from distal movement due to the contact between the central portion 1256 and the on-skin wearable housing 962. The outer portion 1258 of the substrate 1252 accordingly may displace distally relative to the central portion 1256. The overlay patch 1259 may advance distally towards the on-skin wearable medical device and the skin 992 of the host as represented in FIG. 72.

The displacement portion 1266 of the substrate 1252 may elongate or increase in size axially in a similar manner as the displacement portion 1114 of the substrate 1102. The applicator 1250 may be removed upon application of the overlay patch 1259.

FIG. 73 illustrates a resulting application of the overlay patch 1259. The overlay patch 1259 may cover the on-skin wearable housing 962 and the patch 922 or underlay patch as shown in FIG. 73. The entirety of the on-skin wearable housing 962 and the patch 922 may be covered. In examples, at least a portion of the on-skin wearable housing 962 or the patch 922 may be covered.

In examples, the on-skin wearable housing 962 may be at a reduced height than shown in FIG. 73. In examples, the on-skin wearable housing 962 may be at the same height of the patch 922 in examples. An applicator as disclosed herein may yet be utilized to apply an overlay patch to at least a portion of such an on-skin wearable medical device, including a housing of such a medical device and/or a patch of such a device.

The features of the examples of FIGS. 69-73 may be utilized solely or in combination with any other example disclosed herein.

Various other configurations of applicators may be utilized in examples.

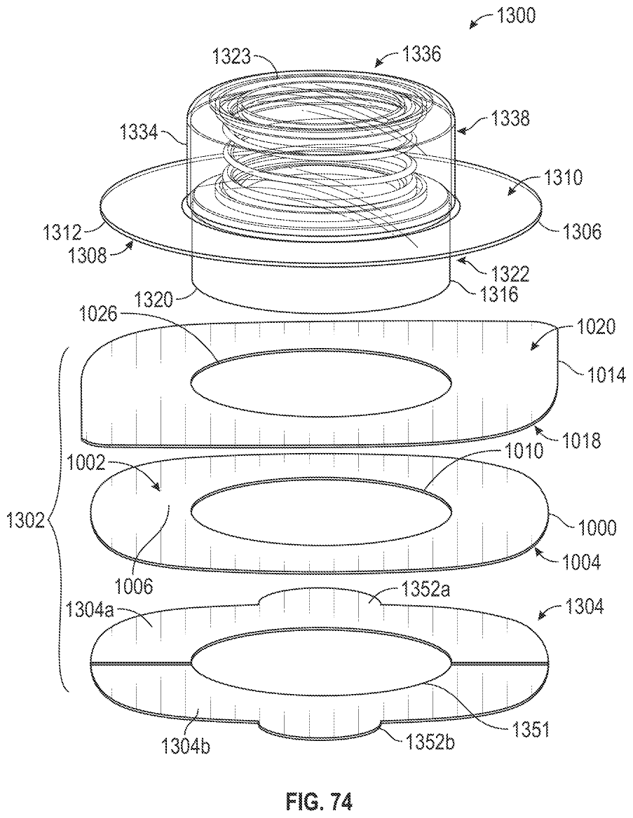

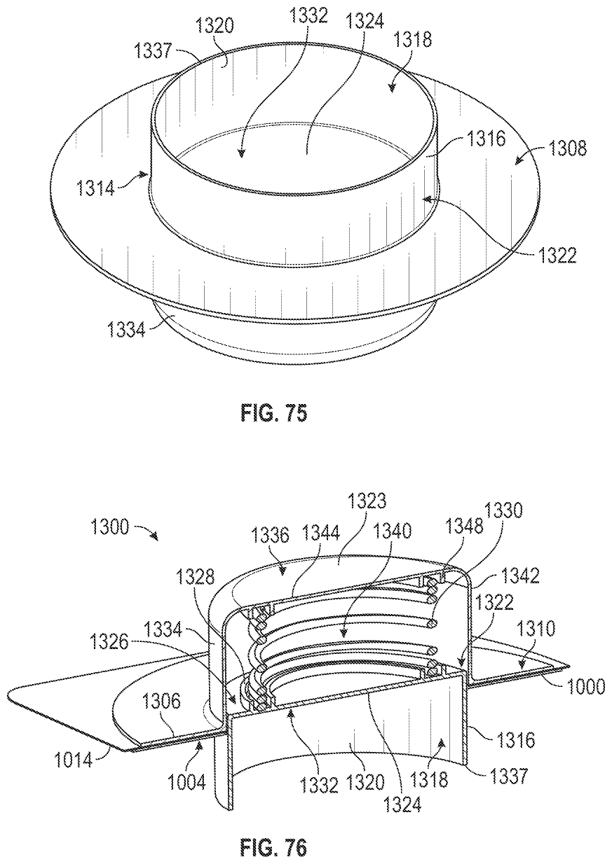

FIG. 74, for example, illustrates a perspective view of an applicator 1300 for supporting an overlay patch 1000 and configured to apply an overlay patch 1000 onto at least a portion of an on-skin wearable medical device when the on-skin wearable medical device is disposed on a skin of the host. FIG. 74 further illustrates a patch assembly 1302 for utilization with examples herein. The patch assembly 1302 may include the overlay patch 1000, the backing layer 1014, and a liner 1304. FIG. 75 illustrates a distal perspective view of the applicator 1300.

The applicator 1300 may include a substrate 1306 (shown in transparency in FIG. 74) for supporting the overlay patch 1000. The substrate 1306, in examples, may comprise a planar surface for the overlay patch 1000 to be positioned upon. The overlay patch 1000 may extend parallel with the planar surface upon being positioned upon the planar surface. The substrate 1306 may comprise a lower or distal surface 1308 (marked in FIG. 75) for the overlay patch 1000 to be positioned upon and may face opposite an upper or proximal surface 1310. In examples, other forms of substrates may be utilized.

The substrate 1306 may include an outer periphery 1312 that may have a diameter or width that is at or less than the diameter or width of the overlay patch 1000 or the backing layer 1014. In examples, however, the outer periphery 1312 that may have a diameter or width that is at or greater than the diameter or width of the overlay patch 1000 or the backing layer 1014. The substrate 1306 accordingly may support the full diameter or width of the overlay patch 1000 or backing layer 1014 to the respective outer periphery of the overlay patch 1000 or backing layer 1014. Other configurations may be utilized in examples.

The substrate 1306 may include a central portion 1314 (marked in FIG. 75). The central portion 1314 (marked in FIG. 75) may correspond to the position of the central portion or overlap portion 1006 of the overlay patch 1000 in examples.

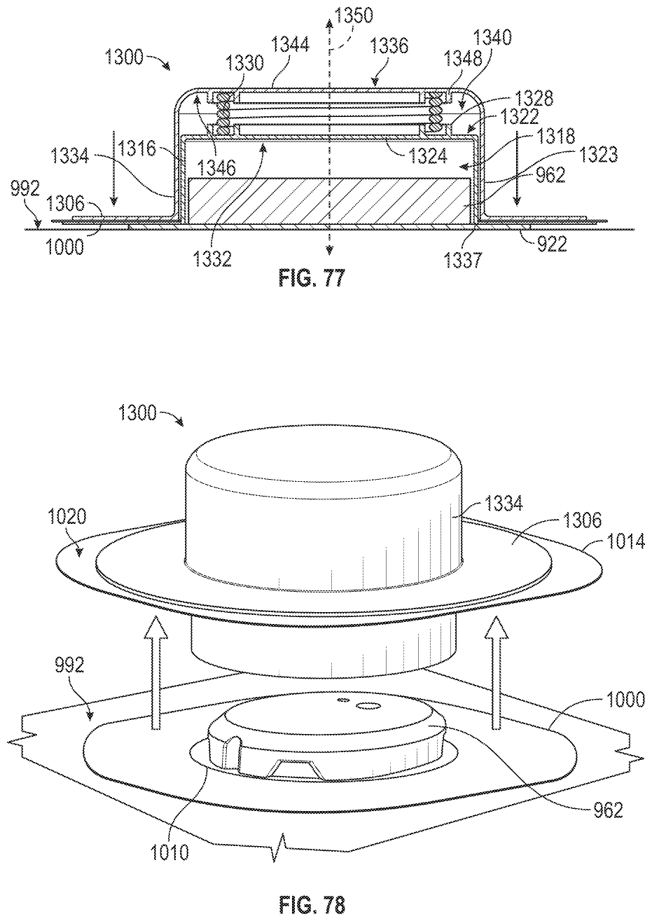

In examples, the applicator 1300 may include an alignment body 1316. The alignment body 1316 may be configured to align the overlay patch 1000 with the on-skin wearable medical device. For example, the alignment body 1316 may orient the applicator 1300 to be positioned at a desired location relative to the on-skin wearable medical device upon application of the overlay patch 1000. The alignment body 1316 may be configured to align with the on-skin wearable housing 962 (marked in FIGS. 77 and 78) in examples. The alignment body 1316 may center the overlay patch 1000 relative to the on-skin wearable housing 962. The overlay patch 1000 accordingly may be disposed to the on-skin wearable medical device with the central opening 1010 of the overlay patch 1000 surrounding the on-skin wearable housing 962. A configuration as shown in FIG. 78 accordingly may result.

The alignment body 1316 may extend from the central portion 1314 of the substrate 1306 in examples. Other positions of the alignment body may be utilized in examples. An intersection of the alignment body 1316 and the substrate 1306 may comprise a displacement portion of the applicator 1300 that may be configured to allow the alignment body 1316 to displace relative to the substrate 1306. The alignment body 1316 may comprise a central portion of the applicator 1300.

The alignment body 1316 may comprise a protrusion. The protrusion may be configured to extend towards the skin of the host upon the applicator 1300 applying the overlay patch 1000 onto at least a portion of the on-skin wearable medical device. The protrusion, in examples, may include a central opening 1318 (marked in FIG. 75) that may be configured to receive a portion of the on-skin wearable medical device. The central opening 1318, for example, may receive the on-skin wearable housing 962. One or more walls 1320 of the protrusion may bound the central opening 1318 and may guide the alignment body 1316 into position around the on-skin wearable housing 962. The protrusion, for example, may comprise a hollow cylinder with the central opening 1318 comprising a channel for receiving a portion of the on-skin wearable medical device (e.g., the on-skin wearable housing 962). The walls 1320 of the protrusion may contact or abut the on-skin wearable housing 962 to align the alignment body 1316 relative to the on-skin wearable medical device. The central opening 1318 and/or the walls 1320 may have a circular shape, as shown in the illustrated example, or may have any other suitable shape, including but not limited to oval, oblong, teardrop, triangular, square, rectangular, rounded square, rounded rectangular, or other suitable curvilinear shape configured to closely or loosely surround an on-skin wearable medical device. In examples, the walls 1320 may continuously surround the opening, while in other examples, the walls may comprise two or more sections spaced apart circumferentially about the opening and cooperating to define the opening.

The overlay patch 1000 may be aligned with the on-skin wearable medical device through use of the alignment body 1316. In examples, the overlay patch 1000 may be aligned with the on-skin wearable medical device prior to the overlay patch 1000 being applied to, or adhered to, the skin.

In examples, the alignment body 1316 may be configured to displace relative to the substrate 1306. For example, the alignment body 1316 may be configured to retract from the skin of the host upon the applicator 1300 applying the overlay patch 1000 onto at least a portion of the on-skin wearable medical device. The alignment body 1316 may be configured to slide relative to the substrate 1306 in examples.

The alignment body 1316, for example, may comprise a portion of a first housing 1322 of the applicator 1300. The first housing 1322 bounds the central opening 1318. The first housing 1322 may include the alignment body 1316 and a proximal or upper wall 1324 (marked in FIG. 76). At least the proximal or upper wall 1324, for example, comprises a displacement body for displacing relative to a second housing 1323. The alignment body 1316 may surround sides of the central opening 1318 and the proximal or upper wall 1324 may bound a proximal or upper end of the central opening 1318. The first housing 1322 may have a cylindrical shape, with the alignment body 1316 comprising one or more curved walls and the proximal or upper wall 1324 comprising a flat or planar wall spanning the upper portion of the one or more curved walls of the alignment body 1316.

A proximal or upper facing surface 1326 (marked in FIG. 76) of the proximal or upper wall 1324 may include a support structure 1328 for supporting a spring 1330. The support structure 1328, for example, may comprise a channel for retaining the spring 1330 or may have another configuration in examples. A distal or lower facing surface 1332 of the proximal or upper wall 1324 may face opposite the upper facing surface 1326 and may face towards the central opening 1318. Other configurations or shapes of the first housing 1322 may be utilized in examples.

The applicator 1300 includes the second housing 1323. The second housing 1323 includes the substrate 1306 and a second displacement body 1334 coupled to the substrate 1306. The second displacement body 1334, for example, may comprise a plunger. The plunger may be configured to be operated by a user to displace the alignment body 1316 relative to the substrate 1306. The plunger may be pressed distally relative to the first housing 1322 to cause the second displacement body 1334 to slide relative to the first housing 1322 and accordingly slide the substrate 1306 relative to the alignment body 1316. The second displacement body 1334 may include a pressing surface 1336 for a user to press to displace the second displacement body 1334 relative to the first housing 1322. The proximal surface 1310 of the substrate 1306 alternatively or in combination may comprise a pressing surface for pressing by a user.

An outer surface 1338 of the second displacement body 1334 may comprise a grip surface or other form of surface for a user to grip upon utilizing the applicator 1300.

In examples, the substrate 1306 may be positioned at the distal end or lower end of the second displacement body 1334. The substrate 1306 may comprise an annular ring, flange, or brim protruding or flaring radially outward from the distal end or lower end of the second displacement body 1334. The substrate 1306 may be positioned to press against a portion of the patch assembly 1302 upon application of the patch 1000.

The second displacement body 1334 of the second housing 1323 may bound a cavity 1340 (marked in FIG. 76) that receives the first housing 1322 of the applicator 1300. The second displacement body 1334 may comprise a sleeve or shroud extending over the first housing 1322 of the applicator 1300. The second displacement body 1334 may include one or more side walls 1342 that bound sides of the cavity 1340 and may include a proximal or upper wall 1344 (marked in FIG. 76) that bounds a proximal or upper end of the cavity 1340. The one or more side walls 1342 may be curved. The second displacement body 1334 may have a cylindrical shape. Other configurations or shapes of the second displacement body 1334 may be utilized in examples.

A lower or distal facing surface 1346 (marked in FIG. 77) of the proximal or upper wall 1344 may face opposite the pressing surface 1336 and towards the cavity 1340. The lower or distal facing surface 1346 may include a support structure 1348 for supporting the spring 1330. The support structure 1348, for example, may comprise a channel for retaining the spring 1330 or may have another configuration in examples. The support structure 1348 may support an opposite end of the spring 1330 than the support structure 1328 (e.g., an upper or proximal end of the spring 1330 supported by the support structure 1348, and a lower or distal end of the spring 1330 supported by the support structure 1328). The spring 1330 may be positioned within the cavity 1340.

The spring 1330 is configured to bias the alignment body 1316 relative to the substrate 1306. The spring 1330 biases the alignment body 1316 to protrude relative to the substrate 1306. The spring 1330 biases the alignment body 1316 to be raised relative to the substrate 1306. The bias may be overcome with a distal force upon the second housing 1323.

Referring to FIG. 77, the applicator 1300 may be configured to deploy the overlay patch 1000 by movement along an axis 1350. The axis 1350 may comprise a central axis that the respective housings 1322, 1323 are centered upon. The axis 1350 may extend through the center of the patch assembly 1302 and/or the patch 1000. Movement along the axis 1350 may cause the respective housings 1322, 1323 to displace relative to each other and cause the substrate 1306 to apply the patch 1000 onto at least the portion of the on-skin wearable medical device. The housing 1322 is configured to slide relative to the housing 1323 along the axis 1350 to apply the patch 1000 onto at least the portion of the on-skin wearable medical device.

The spring 1330 biases the first housing 1322 relative to the second housing 1323. The spring 1330 may comprise a coil spring that extends circumferentially about the axis 1350. The configuration of the spring 1330 may provide for improved force application between the first housing 1322 and the second housing 1323. The spring 1330 may coil in a helical manner about a peripheral portion of the cavity 1340.

Referring to FIG. 74, the patch assembly 1302 is illustrated with the layers of the patch assembly 1302 separated. The upper or proximal surface 1002 of the patch 1000 is configured to be covered with the backing layer 1014. The lower, distal, or engaging surface 1004 of the patch 1000 is configured to be covered by the liner 1304.

The liner 1304 may include two or more liner sections 1304a, b that may cover the lower, distal, or engaging surface 1004 of the patch 1000. The two or more liner sections 1304a, b may form a central opening 1351 of the liner 1304. The liner sections 1304a, b may have respective removal portions 1352a, b for removal of the liner sections 1304a, b from the patch 1000. The removal portions 1352a, b may comprise tabs or wings that extend outward from the respective liner sections 1304a, b. The removal portions 1352a, b are configured to be grasped by a user and pulled to remove the respective liner sections 1304a, b. In examples, other configurations as disclosed herein, including use of a liner removal assembly, may be utilized. The liner 1304 may otherwise include the features as described regarding the liner 1016 or any other liner disclosed herein.

In examples, the proximal surface 1020 of the backing layer 1014 may comprise an adhesive surface for coupling to the distal surface 1308 of the substrate 1306. All or a portion of the proximal surface 1020 may comprise an adhesive surface as desired. The distal surface 1018 of the backing layer 1014 may comprise an adhesive surface for coupling to the upper or proximal surface 1002 of the overlay patch 1000. Other configurations of backing layers may be utilized in examples as desired. In examples, the distal surface 1308 of the substrate 1306 may comprise an adhesive surface for coupling with at least a portion of the patch assembly 1302.

The patch assembly 1302 may be positioned upon the substrate 1306 in examples. The patch 1000 may be layered between the backing layer 1014 and the liner 1304. The backing layer 1014 may be layered between the patch 1000 and the substrate 1306. The alignment body 1316 extends through the central openings 1010, 1026, 1351 of the respective overlay patch 1000, backing layer 1014, and the liner 1304. The alignment body 1316 may align the overlay patch 1000 upon the substrate 1306 (e.g., guide the overlay patch 1000 to a defined position upon the substrate 1306). The overlay patch 1000 may be positioned upon the substrate 1306 with the lower, distal, or engaging surface 1004 facing away from the substrate 1306 and in a distal direction towards the skin of the host.

FIGS. 76-78 illustrate an exemplary application procedure of the overlay patch 1000 utilizing the applicator 1300. Steps of the application procedure may be varied, excluded, or substituted as desired. Combinations of features of procedures may be used in combination or substitution as desired.

Referring to FIG. 76, a cross sectional view of the overlay patch 1000 and the backing layer 1014 positioned upon the substrate 1306 is shown. A user (such as the host) may position such components upon the substrate 1306 with the liner 1304 attached. A user may then remove the liner 1304 while the overlay patch 1000 and backing layer 1014 are positioned upon the substrate 1306 to result in the configuration shown in FIG. 76. The engaging surface 1004 of the overlay patch 1000 is exposed. In examples, the liner 1304 may be removed prior to the overlay patch 1000 and backing layer 1014 being positioned upon the substrate 1306.

The on-skin wearable medical device may be disposed upon skin of the host. The applicator having the overlay patch 1000 and backing layer 1014 coupled thereto may approach the on-skin wearable medical device. The exposed engaging surface 1004 of the overlay patch 1000 faces towards the skin and the on-skin wearable medical device.

The alignment body 1316 may be utilized to align the overlay patch 1000 with the on-skin wearable medical device. The on-skin wearable housing 962 may be positioned within the central opening 1318 of the alignment body 1316 to align the overlay patch 1000 with or around the on-skin wearable medical device. A leading edge 1337 of the alignment body 1316 may be positioned upon the patch 922 or underlay patch surrounding the on-skin wearable housing 962 (as represented in FIG. 77). The alignment body 1316 may guide the applicator 1300 to a desired position. A tactile indication of the position of the alignment body 1316 relative to the on-skin wearable medical device may be provided by a feel of the on-skin wearable housing 962 being inserted into the central opening 1318, and/or by a feel of the leading edge 1337 making contact with the skin of the patient around the on-skin wearable housing 962, either directly or indirectly through the patch 922 or underlay patch surrounding the on-skin wearable housing 962. Such tactile indication may be beneficial if the on-skin wearable medical device is in a position that is difficult to visualize (e.g., on a back surface of an arm or other position).

Referring to FIG. 77, the second displacement body 1334 or plunger may be pressed in a direction towards the skin 992 of the host. The movement of the second displacement body 1334 relative to the first housing 1322 may cause the substrate 1306 to displace distally relative to the alignment body 1316. The overlay patch 1000 may advance distally towards the on-skin wearable medical device and the skin 992 of the host. The second displacement body 1334 or plunger may continue to be pressed in a direction towards the skin 992 of the host until the overlay patch 1000 contacts at least a portion of the on-skin wearable medical device (for example, the patch 922) and/or the skin 992, preferably while avoiding contact between the patch 1000 and the on-skin wearable housing 962. Optionally, a pressure applied by a user at this stage to the overlay patch 1000 may allow the overlay patch 1000 to engage the skin positioned outward of the patch 922 or underlay patch. A pressure applied by the user may further engage the overlay patch 1000 to the on-skin wearable medical device, e.g., to the patch 922.

Referring to FIG. 78, the applicator 1300 may be removed or withdrawn from the skin. The overlay patch 1000 may separate from the applicator 1300 upon removal of the applicator 1300 due to the adhesion or other engagement between the overlay patch 1000 and the on-skin wearable medical device (e.g., the patch 922) or skin. The backing layer 1014 may remain adhered to the substrate 1306 due to the proximal surface 1020 of the backing layer 1014 comprising an adhesive surface. In examples, the proximal surface 1020 may lack an adhesive and the backing layer 1014 may be separately removed from the overlay patch 1000 after application of the overlay patch 1000 and removal of the applicator 1300. In examples, the use of a backing layer 1014 may be excluded, or a backing layer 1014 may be removed prior to positioning of the overlay patch 1000 upon the substrate 1306 of the applicator 1300.

Upon removal of the applicator 1300, the overlay patch 1000 may be pressed or smoothed by a user (such as the host) to further apply the overlay patch 1000 to the on-skin wearable medical device or the skin 992. A pressure applied by a user may allow the overlay patch 1000 to engage the skin positioned outward of the patch 922 or underlay patch. A pressure applied by the user may further engage the overlay patch 1000 to the on-skin wearable medical device.

FIG. 78 illustrates a resulting configuration of the overlay patch 1000 applied onto the patch 922 or underlay patch of the on-skin wearable medical device, and applied to the skin 992 of the host.

The use of the applicator may beneficially improve the case upon which the overlay patch may be applied to a desired application site.

The features of the examples of FIGS. 74-78 may be utilized solely or in combination with any other example disclosed herein.

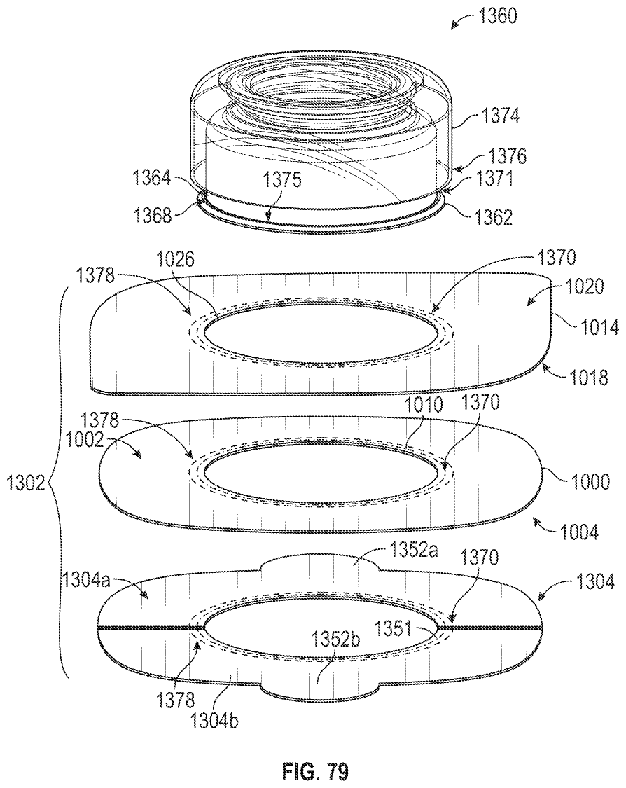

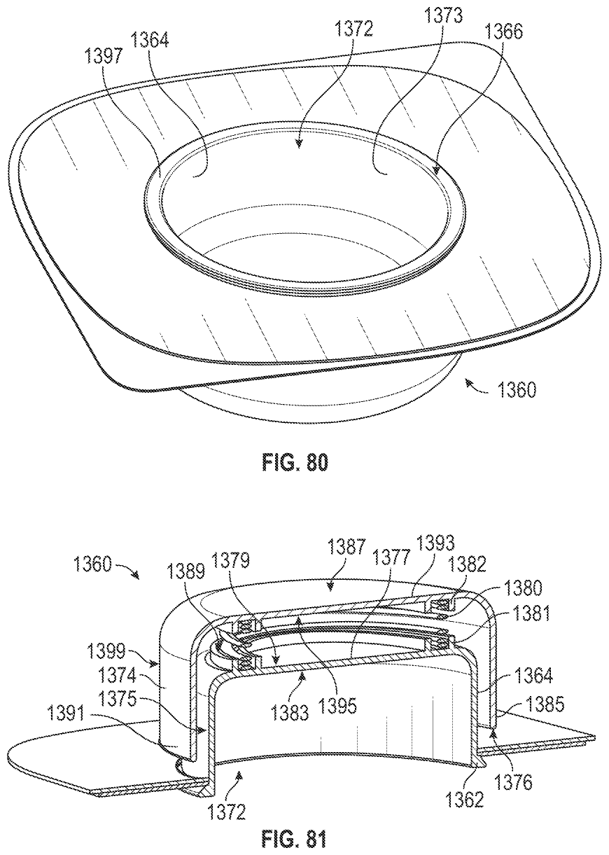

Various other configurations of applicators may be utilized in examples. FIG. 79, for example, illustrates a perspective view of an applicator 1360 for supporting an overlay patch 1000 and configured to apply an overlay patch 1000 onto at least a portion of an on-skin wearable medical device when the on-skin wearable medical device is disposed on a skin of the host. FIG. 79 further illustrates the patch assembly 1302 for utilization with examples herein. FIG. 80 illustrates a distal perspective view of the applicator 1360 with layers of the patch assembly 1302 coupled thereto. FIG. 81 illustrates a perspective cross sectional view of the applicator 1360 with layers of the patch assembly 1302 coupled thereto.

The applicator 1360 may include a substrate 1362 for supporting the overlay patch 1000. The substrate 1362 may comprise a ridge or protruding rim in examples. The ridge may protrude radially outward from a housing comprising the alignment body 1364. The ridge may extend circumferentially about the entirety of the alignment body 1364. In other examples, the ridge may comprise two or more sections spaced apart circumferentially about the alignment body 1364. The ridge may be positioned at a distal end of the alignment body 1364 and at the leading edge 1397 of the alignment body 1364 (marked in FIG. 80). In examples, other forms of substrates may be utilized.

The ridge may include a lower or distal surface 1366 (marked in FIG. 80) and may include an opposite upper or proximal surface 1368. The upper or proximal surface 1368 may support the overlay patch 1000 or at least a portion of the patch assembly 1302. The overlay patch 1000 or at least a portion of the patch assembly 1302 may be positioned upon the upper or proximal surface 1368 of the ridge.

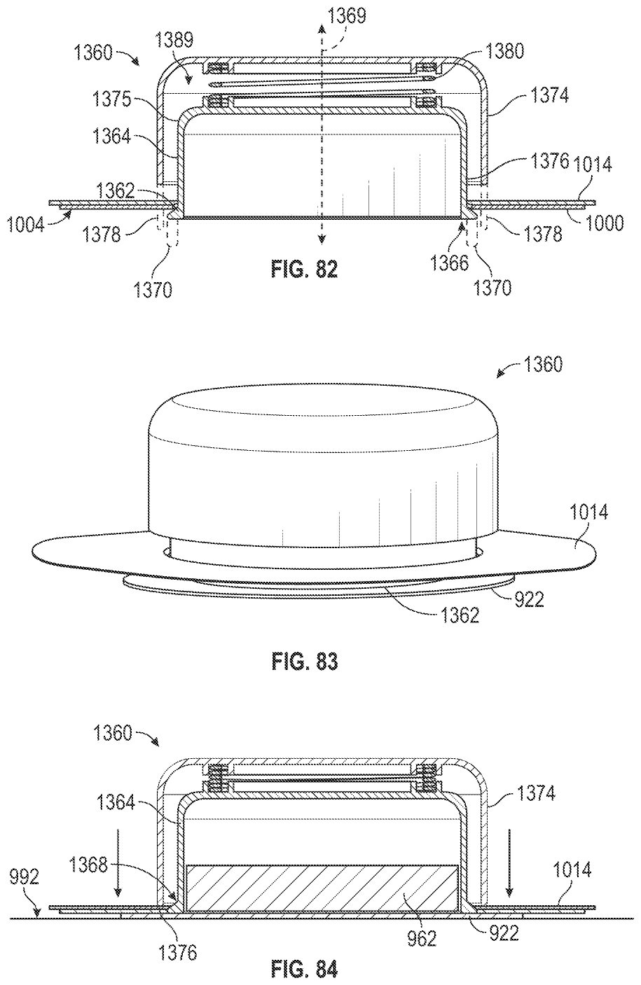

In examples, the lower or distal surface 1366 of the ridge may extend perpendicular to an axis 1369 (marked in FIG. 82) that the applicator 1360 moves along to deploy the overlay patch 1000 to the on-skin wearable medical device. The upper or proximal surface 1368 of the ridge may be angled with respect to the axis 1369, such that the ridge is wider at a distal end of the ridge than at a proximal end of the ridge. The ridge may include a central portion 1371. The axial thickness of the ridge is greater at the central portion 1371 or radially inward portion of the ridge as compared to a peripheral portion or radially outward portion of the ridge. The downward angle or taper of the ridge may allow for improved release of the overlay patch 1000 or the portion of the patch assembly 1302 from the ridge. The ridge may be configured to disengage from the patch 1000 upon application of the patch 1000 onto at least the portion of the on-skin wearable medical device.

The ridge may be configured to engage an interior annular section 1370 (marked with dot-dash lines in FIG. 79) of at least a portion of the patch assembly 1302. The interior annular section 1370 may be bounded at a radial interior by the respective central openings 1026, 1010, 1351. The ridge may protrude radially outward to directly support only a portion of the patch assembly 1302. The ridge may engage and support the interior annular section 1370 as shown in FIG. 82 for example. As such, only a portion of the patch assembly 1302 is supported by the substrate 1362, with the radially outward portion, or peripheral portion, of the patch assembly 1302 not being directly supported upon the substrate 1362.

The applicator 1360 may include the alignment body 1364. The alignment body 1364 may comprise a protrusion and may be positioned at the central portion 1371 of the ridge or substrate 1362. The protrusion may be configured to extend towards the skin of the host upon the applicator 1360 applying the overlay patch 1000 onto at least a portion of the on-skin wearable medical device. The alignment body 1364 may comprise a central portion of the applicator 1360. The alignment body 1364 may be configured to align the applicator 1360 with the on-skin wearable medical device in a similar manner as with the alignment body 1316.

For example, referring to FIG. 80, the alignment body 1364 includes a central opening 1372 for receiving a portion of the on-skin wearable medical device for alignment with the on-skin wearable medical device. One or more walls 1373 of the alignment body 1364 may bound the central opening 1372 and may guide the alignment body 1364 into position around the on-skin wearable housing 962 (as marked in FIG. 84). The alignment body 1364, for example, may comprise a hollow cylinder with the central opening 1372 comprising a channel for receiving a portion of the on-skin wearable medical device (e.g., the on-skin wearable housing 962). The walls 1373 of the protrusion may contact or abut the on-skin wearable housing 962 to align the alignment body 1364 relative to the on-skin wearable medical device. The overlay patch 1000 accordingly may be aligned with the on-skin wearable medical device.

The alignment body 1364 may comprise a portion of a first housing 1375 of the applicator 1360. The first housing 1375 bounds the central opening 1372. The first housing 1375 may include the alignment body 1364 and a proximal or upper wall 1377 (marked in FIG. 81), and the substrate 1362 at a lower or distal end of the first housing 1375. At least the proximal or upper wall 1377, for example, comprises a displacement body for displacing relative to a second housing 1374. The alignment body 1364 may surround sides of the central opening 1372 and the proximal or upper wall 1377 may bound a proximal or upper end of the central opening 1372. The first housing 1375 may have a cylindrical shape, with the alignment body 1364 comprising one or more curved walls and the proximal or upper wall 1377 comprising a flat or planar wall spanning the upper portion of the one or more curved walls of the alignment body 1364.

A proximal or upper facing surface 1379 (marked in FIG. 81) of the proximal or upper wall 1377 may include a support structure 1381 for supporting a spring 1380. The support structure 1381, for example, may comprise a channel for retaining the spring 1380 or may have another configuration in examples. A distal or lower facing surface 1383 of the proximal or upper wall 1377 may face opposite the upper facing surface 1379 and may face towards the central opening 1372. Other configurations or shapes of the first housing 1375 may be utilized in examples.

The applicator 1360 may include a second housing 1374 for sliding relative to the first housing 1375. The second housing 1374 may include a pressure surface 1376 and a second displacement body 1385 coupled to the pressure surface 1376. The second displacement body 1385, for example, may comprise a plunger. The plunger may be configured to be operated by a user to displace the second displacement body 1385 and pressure surface 1376 relative to the first housing 1375. The plunger may be pressed distally relative to the first housing 1375 to cause the second displacement body 1385 to slide relative to the first housing 1375 and accordingly slide the pressure surface 1376 relative to the first housing 1375. The second displacement body 1385 may include a pressing surface 1387 for a user to press to displace the second displacement body 1385 relative to the first housing 1375.

The second displacement body 1385 of the second housing 1374 may bound a cavity 1389 that receives the first housing 1375 of the applicator 1360. The second displacement body 1385 may comprise a sleeve or shroud extending over the first housing 1375 of the applicator 1360. The second displacement body 1385 may include one or more side walls 1391 that bound sides of the cavity 1389 and may include a proximal or upper wall 1393 (marked in FIG. 81) that bounds a proximal or upper end of the cavity 1389. The one or more side walls 1391 may be curved. The second displacement body 1385 may have a cylindrical shape. An outer surface 1399 of the second displacement body 1385 may comprise a grip surface or other form of surface for a user to grip upon utilizing the applicator 1360. Other configurations or shapes of the second displacement body 1385 may be utilized in examples.

A lower or distal facing surface 1395 (marked in FIG. 81) of the proximal or upper wall 1393 may face opposite the pressing surface 1387 and towards the cavity 1389. The lower or distal facing surface 1395 may include a support structure 1382 for supporting the spring 1380. The support structure 1382, for example, may comprise a channel for retaining the spring 1380 or may have another configuration in examples. The support structure 1382 may support an opposite end of the spring 1380 than the support structure 1381 (e.g., an upper or proximal end of the spring 1380 supported by the support structure 1382, and a lower or distal end of the spring 1380 supported by the support structure 1381). The spring 1380 may be positioned within the cavity 1389.

An intersection of the first housing 1375 and the second housing 1374 may comprise a displacement portion of the applicator 1360 that may be configured to allow the first housing 1375 to displace relative to the second housing 1374. For example, the alignment body 1364 may be configured to retract from the skin of the host upon the applicator 1360 applying the overlay patch 1000 onto at least a portion of the on-skin wearable medical device. The first housing 1375 may be configured to slide relative to the second housing 1374 in examples.

Referring to FIG. 81, a spring 1380 may be disposed within a cavity 1389 to bias the first housing 1375 relative to the second housing 1374. The spring 1380 biases the alignment body 1364 to protrude relative to the second housing 1374. The spring 1380 biases the alignment body 1364 to be raised relative to the pressure surface 1376 of the second housing 1374. The bias may be overcome with a distal force upon the second housing 1374.

Referring to FIG. 82, the applicator 1360 may be configured to deploy the overlay patch 1000 by movement along an axis 1369. The axis 1369 may comprise a central axis that the respective housings 1374, 1375 are centered upon. The axis 1369 may extend through the center of the patch assembly 1302 and/or the patch 1000. Movement along the axis 1369 may cause the respective housings 1374, 1375 to displace relative to each other and cause the patch 1000 to be applied onto at least the portion of the on-skin wearable medical device. The housing 1374 is configured to slide relative to the housing 1375 along the axis 1369 to apply the patch 1000 onto at least the portion of the on-skin wearable medical device.

The spring 1380 may comprise a coil spring that extends circumferentially about the axis 1369. The spring 1380 biases the first housing 1375 relative to the second housing 1374. The configuration of the spring 1380 may provide for improved force application between the first housing 1375 and the second housing 1374. The spring 1380 may coil in a helical manner about a peripheral portion of the cavity 1389.

The second housing 1374 may include a pressure surface 1376 for pressing at least a portion of the patch assembly 1302 towards the skin of the host. The pressure surface 1376 may press at least a portion of the patch assembly 1302 towards the skin of the host to disengage the interior annular section 1370 (marked in FIG. 79) from the substrate 1362. The pressure surface 1376 may press a section 1378 (marked in FIG. 79) of the patch assembly 1302 that is positioned radially outward of the interior annular section 1370, with the section 1378 being bound between the dot-dash lines and the dash lines in FIG. 79. The section 1378 may comprise a pressing section forming a ring or annulus positioned radially outward of the interior annular section 1370.

The pressure surface 1376 may have an annular shape. The pressure surface 1376 may press a portion of the patch assembly 1302 at a location radially outward of the substrate 1362 or ridge.

The pressure surface 1376 may press at least a portion of the patch assembly 1302 to disengage the patch assembly 1302 from the substrate 1362 or ridge. The pressure surface 1376 may further press the patch assembly 1302 onto the portion of the on-skin wearable medical device to couple or adhere at least a portion of the patch assembly 1302 (e.g., the patch 1000) onto the portion of the on-skin wearable medical device (e.g., onto the patch 922), preferably while avoiding contact between the patch 1000 and the on-skin wearable housing 962.

FIGS. 82-84 illustrate an exemplary deployment of the overlay patch 1000 to at least the portion of the on-skin wearable medical device. Referring to FIG. 82, the patch assembly 1302 may be positioned upon the substrate 1362 or ridge by being slid in a proximal or upper direction over the substrate 1362, such that the patch assembly 1302 rests upon the upper or proximal surface 1368 of the substrate 1362. Alternatively, the second housing 1374 may be separated from the alignment body 1364 and the patch assembly 1302 may be slid over the alignment body 1364 in a distal direction to rest upon the upper or proximal surface 1368 of the substrate 1362. The second housing 1374 may then be rejoined with the alignment body 1364 with the spring 1380 positioned therebetween.

The patch 1000 may be layered between the backing layer 1014 and the liner 1304. The patch 1000 may be layered between the backing layer 1014 and the substrate 1362. The alignment body 1364 extends through the central openings 1010, 1026, 1351 of the respective overlay patch 1000, backing layer 1014, and the liner 1304. The alignment body 1364 may align the overlay patch 1000 upon the substrate 1362 (e.g., guide the overlay patch 1000 to a defined position upon the substrate 1362). The overlay patch 1000 may be positioned upon the substrate 1362 with the lower, distal, or engaging surface 1004 facing in a distal direction towards the skin of the host.

With the patch assembly 1302 upon the substrate 1362, the liner 1304 may be removed such that the engaging surface 1004 of the patch 1000 is exposed (as shown in FIG. 82). In examples, the liner 1304 may be removed prior to positioning the patch 1000 and backing layer 1014 upon the substrate 1362.

The on-skin wearable medical device may be disposed upon skin of the host. The applicator 1360 may then approach the on-skin wearable medical device. The alignment body 1364 may align with the housing of the on-skin wearable medical device for positioning of the patch 1000 relative to the on-skin wearable medical device. The on-skin wearable housing 962 may be positioned within the central opening 1372 of the alignment body 1364 to align the overlay patch 1000 with the on-skin wearable medical device. A leading edge 1397 (marked in FIG. 80) of the alignment body 1364 may be positioned upon the patch 922 or underlay patch surrounding the on-skin wearable housing 962 (as represented in FIG. 83). The alignment body 1364 may guide the applicator 1360 to a desired position. A tactile indication of the position of the alignment body 1364 relative to the on-skin wearable medical device may be provided by a feel of the on-skin wearable housing 962 being inserted into the central opening 1372. Such tactile indication may be beneficial if the on-skin wearable medical device is in a position that is difficult to visualize (e.g., on a back surface of an arm or other position).

A configuration as shown in FIG. 83 may result. The substrate 1362 or ridge may suspend the patch 1000 at a distance from the patch 922 of the on-skin wearable medical device.

Referring to FIG. 84, a user may then press the second housing 1374 distally relative to the alignment body 1364. The pressure surface 1376 of the second housing 1374 may press at least a portion of the patch assembly 1302 distally to disengage the patch assembly 1302 from the substrate 1362 or ridge. For example, the patch assembly 1302 may deflect to release from the substrate 1362 or ridge. The tapered shape of the upper or proximal surface 1368 of the substrate 1362 may assist to release the patch assembly 1302 from the substrate 1362 or ridge.

The movement of the pressure surface 1376 of the second housing 1374 may adhere or engage the patch 1000 to the on-skin wearable medical device (e.g., to the patch 922), preferably while avoiding contact between the patch 1000 and the on-skin wearable housing 962. Optionally, a user may manually smooth and press the patch 1000 onto the patch 922 of the on-skin wearable medical device and/or the skin 992 of the host at this stage.

The applicator 1360 may then be removed from the on-skin wearable medical device. A user may manually smooth and press the patch 1000 onto the patch 922 of the on-skin wearable medical device and/or the skin 992 of the host. The backing layer 1014 may be removed by the user. Alternatively, use of the backing layer 1014 may be excluded and the patch 1000 may be solely supported upon the substrate 1362 or ridge.

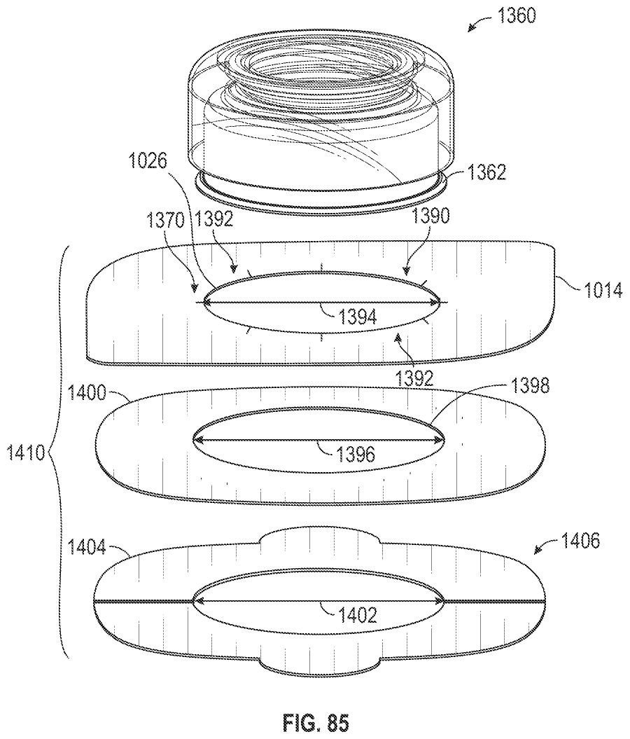

FIGS. 85-87 illustrate a variation in which the backing layer 1014 includes one or more cuts or perforations 1390 configured to allow for disengagement of the backing layer 1014 from the substrate 1362 or ridge. The cuts or perforations 1390 may be positioned at the interior annular section 1370 of the backing layer 1014. The cuts or perforations 1390 may extend radially outward to allow for displacement of portions 1392 of the backing layer 1014 positioned between the respective cuts or perforations 1390.

The backing layer 1014 may have an interior diameter 1394 of the central opening 1026 that is less than an interior diameter 1396 of the central opening 1398 of the patch 1400 (or overlay patch 1400). The interior diameter 1394 of the central opening 1026 of the backing layer 1014 may further be less than the interior diameter 1402 of the central opening 1404 of the liner 1406. The interior annular section 1370 of the backing layer 1014 accordingly may protrude radially inward from the central opening 1398 of the patch 1400 and the central opening 1404 of the liner 1406 upon assembly of the patch assembly 1410. The patch 1400 may otherwise be configured similarly as the patch 1000 or other patches disclosed herein. The liner 1406 may otherwise be configured similarly as the liner 1304 or other liners disclosed herein.

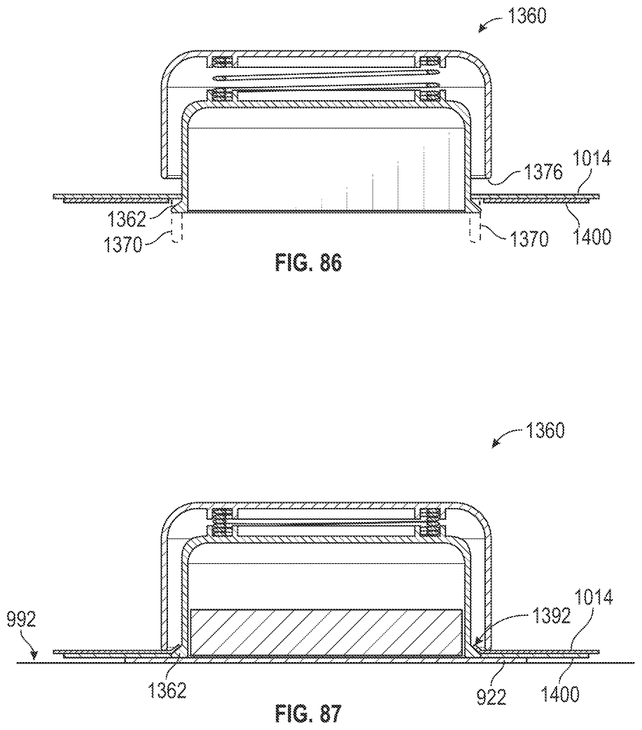

FIG. 86 illustrates a configuration utilizing the patch assembly 1410 with the interior annular section 1370 of the backing layer 1014 being supported upon the substrate 1362 or ridge, and the central opening 1398 of the patch 1400 being sized such that an inner edge of the patch 1400 is positioned radially outward of the substrate 1362 or ridge. As such, the substrate 1362 or ridge directly supports only a portion of the patch assembly 1410 (e.g., the backing layer 1014). The substrate 1362 accordingly supports an interior annular section of a patch assembly including one or more of the patch 1400, a liner 1406 for the patch 1400, or a backing layer 1014 for the patch 1400. One or more layers of the patch assembly may be supported in examples herein.

A pressing movement of the pressure surface 1376 presses radially outward of the interior annular section 1370 of the backing layer 1014 as represented in FIG. 87. The cuts or perforations 1390 in the backing layer 1014 allow for deflection of the backing layer 1014 at the interior annular section 1370, yet the overlay patch 1400 remains undeflected.

A user may remove the applicator 1360 and simultaneously remove the backing layer 1014 due to the contact between the backing layer 1014 with the substrate 1362 or ridge. Alternatively, the user may remove the applicator 1360 and separately remove the backing layer 1014. A user may manually smooth and press the patch 1400 onto the patch 922 of the on-skin wearable medical device and/or the skin 992 of the host. In examples, this smoothing and pressing may be performed before and/or after removal of the applicator 1360.

The features of the examples of FIGS. 79-87 may be utilized solely or in combination with any other example disclosed herein.

Various other configurations of applicators may be utilized in examples.

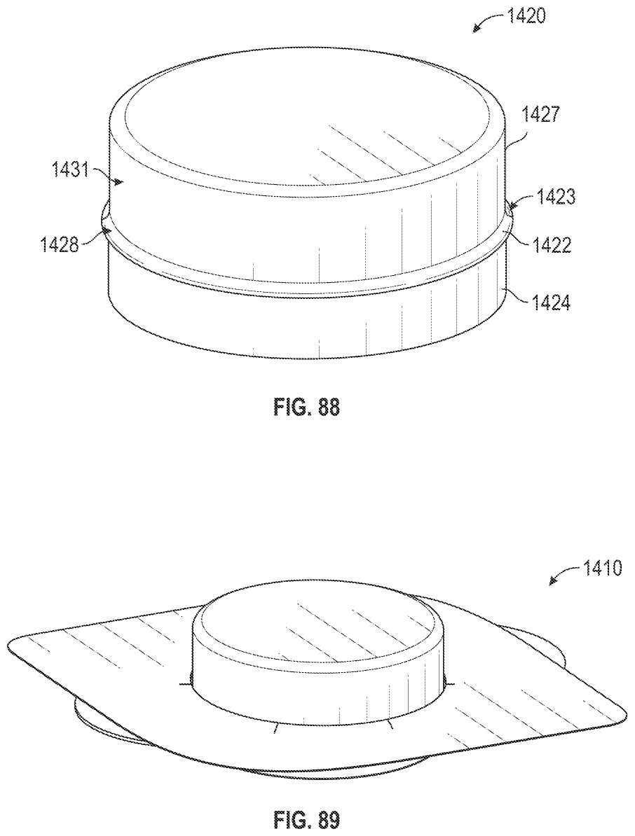

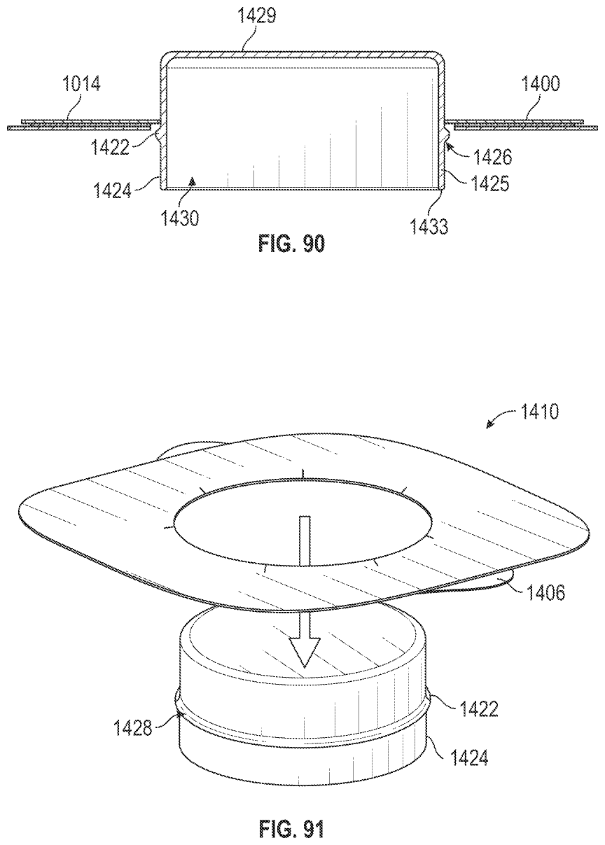

FIG. 88, for example, illustrates a perspective view of an applicator 1420 for supporting an overlay patch and configured to apply an overlay patch onto at least a portion of an on-skin wearable medical device when the on-skin wearable medical device is disposed on a skin of the host. FIG. 89 illustrates a perspective view of the applicator 1420 with a patch assembly coupled thereto. FIG. 90 illustrates a cross sectional view of the configuration shown in FIG. 89.

The applicator 1420 may include a substrate 1422 for supporting the overlay patch 1400. The substrate 1422 may comprise a ridge or protruding rim. The ridge may protrude radially outward from a housing comprising the alignment body 1424. The ridge may extend circumferentially about the entirety of the alignment body 1424. In other examples, the ridge may comprise two or more sections spaced apart circumferentially about the alignment body 1424. The ridge may be positioned at a central portion of the alignment body 1424 (positioned axially central relative to the proximal end and distal end of the alignment body 1424). The ridge may be spaced from the leading edge 1433 (marked in FIG. 90) of the alignment body 1424. In examples, other forms of substrates may be utilized.

The ridge may include a lower or distal surface 1426 (marked in FIG. 90) and may include an opposite upper or proximal surface 1428. The upper or proximal surface 1428 may support the overlay patch 1400 or at least a portion of the patch assembly 1410. The overlay patch 1400 or at least a portion of the patch assembly 1410 may be positioned upon the upper or proximal surface 1428 of the ridge.

In examples, the lower surface 1426 and upper surface 1428 may each have a rounded shape. The rounded shape may allow for improved release or disengagement of the overlay patch 1400 or the portion of the patch assembly 1410 from the ridge. The ridge may be configured to disengage from the patch 1400 upon application of the patch 1400 onto at least the portion of the on-skin wearable medical device.

The ridge may be configured to engage an interior annular section of at least a portion of the patch assembly 1410 in a similar manner as with the substrate 1362. The patch assembly 1410 may rest upon the ridge as shown in FIG. 90. The ridge may protrude radially outward to directly support only a portion of the patch assembly 1410. The ridge may engage and support the interior annular section as shown in FIG. 90 for example. As such, only a portion of the patch assembly 1410 is supported by the substrate 1422, with the radially outward portion of the patch assembly 1410 not being directly supported upon the substrate 1422.

The applicator 1420 may include the alignment body 1424 for aligning the applicator 1420 with the on-skin wearable medical device in a similar manner as with the alignment body 1316. The alignment body 1424 may comprise a protrusion and may be positioned at the central portion 1423 of the ridge or substrate 1422. The protrusion may be configured to extend towards the skin of the host upon the applicator 1420 applying the overlay patch 1400 onto at least a portion of the on-skin wearable medical device. The alignment body 1424 may comprise a central portion of the applicator 1420.

For example, referring to FIG. 90, the alignment body 1424 includes a central opening 1430 for receiving a portion of the on-skin wearable medical device for alignment with the on-skin wearable medical device.

One or more walls 1425 (marked in FIG. 90) of the alignment body 1424 may bound the central opening 1430 and may guide the alignment body 1424 into position around the on-skin wearable housing 962. The alignment body 1424, for example, may comprise a hollow cylinder with the central opening 1430 comprising a channel for receiving a portion of the on-skin wearable medical device (e.g., the on-skin wearable housing 962). The walls 1425 of the protrusion may contact or abut the on-skin wearable housing 962 to align the alignment body 1424 relative to the on-skin wearable medical device. The overlay patch 1400 accordingly may be aligned with the on-skin wearable medical device.

The alignment body 1424 may comprise a portion of a housing 1427 of the applicator 1420. The housing 1427 may include the alignment body 1424 and a proximal or upper wall 1429 (marked in FIG. 90), and the substrate 1422 at the central portion of the alignment body 1424. The alignment body 1424 may surround sides of the central opening 1430 and the proximal or upper wall 1429 may bound a proximal or upper end of the central opening 1430. The housing 1427 may have a cylindrical shape, with the alignment body 1424 comprising one or more curved walls and the proximal or upper wall 1429 comprising a flat or planar wall spanning the upper portion of the one or more curved walls of the alignment body 1424. An outer surface 1431 of the housing 1427 may comprise a grip surface or other form of surface for a user to grip upon utilizing the applicator 1420.

FIGS. 91-93 illustrate an exemplary deployment of the overlay patch 1400 to at least the portion of the on-skin wearable medical device. Referring to FIG. 91, the patch assembly 1410 may be positioned upon the substrate 1422 or ridge by being slid in a distal or lower direction along the alignment body 1424 towards the substrate 1422. The patch assembly 1410 may rest upon the upper or proximal surface 1428 of the substrate 1422. In examples, a configuration of the patch assembly 1410 as shown in FIG. 85 may be utilized, in which the interior annular section 1370 of the backing layer 1014 may rest upon the substrate 1422, with the inner edge of the overlay patch 1400 being positioned radially outward of the substrate 1422. FIG. 90 represents such a configuration. In examples, a different configuration (e.g., a configuration of the patch assembly 1302 may be utilized).

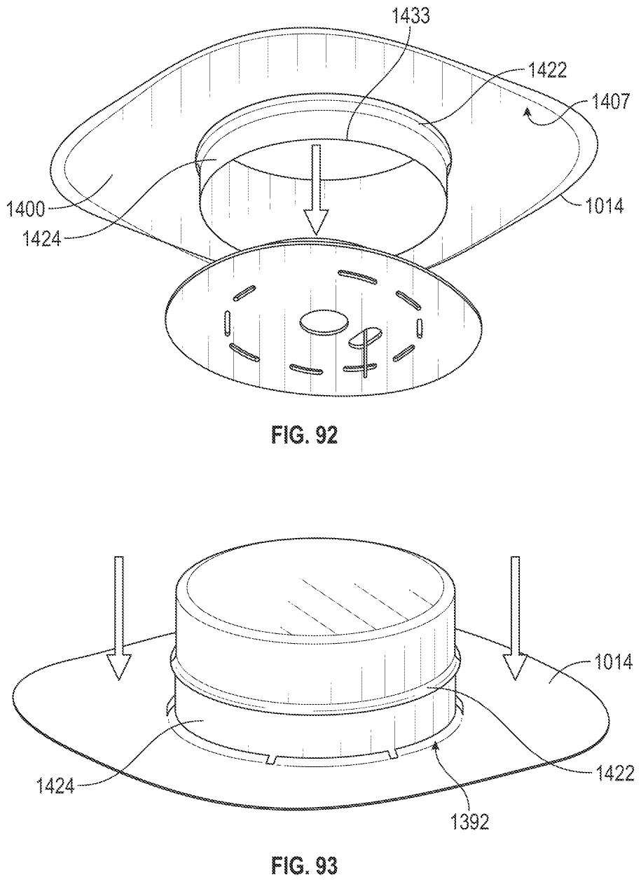

With the patch assembly 1410 upon the substrate 1422, the liner 1406 may be removed such that the engaging surface 1407 of the patch 1400 is exposed (as shown in FIG. 92). In examples, the liner 1406 may be removed prior to positioning the patch 1400 and backing layer 1014 upon the substrate 1422.

The applicator 1420 may then approach the on-skin wearable medical device. FIG. 92 illustrates an approach of the applicator to the on-skin wearable medical device. The alignment body 1424 may align with the housing of the on-skin wearable medical device for positioning of the patch 1400 relative to the on-skin wearable medical device. The on-skin wearable housing 962 may be positioned within the central opening 1430 of the alignment body 1424 to align the overlay patch 1400 with the on-skin wearable medical device. A leading edge 1433 of the alignment body 1424 may be positioned upon the patch 922 or underlay patch surrounding the on-skin wearable housing 962. The alignment body 1424 may guide the applicator 1420 to a desired position. A tactile indication of the position of the alignment body 1424 relative to the on-skin wearable medical device may be provided by a feel of the on-skin wearable housing 962 being inserted into the central opening 1430. Such tactile indication may be beneficial if the on-skin wearable medical device is in a position that is difficult to visualize (e.g., on a back surface of an arm or other position).

The substrate 1422 or ridge comprises a datum feature, or staging area, for setting a distance of the patch 1400 from the leading edge 1433 of the alignment body 1424 prior to application of the patch 1400 onto at least the portion of the on-skin wearable medical device. As such, the spacing or distance allows for alignment with the on-skin wearable medical device without the patch 1400 undesirably adhering to the on-skin wearable medical device prior to the desired time of deployment. As such, the substrate 1422 or ridge retains the patch 1400 at the spacing or distance prior to the desired time of deployment.

At a desired time of deployment, the user may press the patch 1400 distally to slide along the alignment body for deployment to the on-skin wearable medical device (e.g., to the patch 922), preferably while avoiding contact between the patch 1400 and the on-skin wearable housing 962. The force applied by the user disengages the patch 1400 and the backing layer 1014 from the substrate 1422 and allows the patch 1400 and backing layer 1014 to slide distally from the substrate 1422. The portions 1392 of the backing layer 1014 are shown deflected to allow the patch 1400 to slide distally.

The applicator 1420 may then be removed from the on-skin wearable medical device. A user may manually smooth and press the patch 1400 onto the patch 922 of the on-skin wearable medical device and/or the skin 992 of the host. In examples, this smoothing and pressing may be performed before and/or after removal of the applicator 1420. The backing layer 1014 may be removed by the user.

In examples, the use of the backing layer 1014 may be excluded and the patch 1400 may be solely supported upon the substrate 1422 or ridge. In such configurations, the interior diameter of the patch 1400 may be sized such that the patch 1400 rests upon the substrate 1422 or ridge.

The features of the examples of FIGS. 88-93 may be utilized solely or in combination with any other example disclosed herein.

Various other configurations of applicators may be utilized in examples.

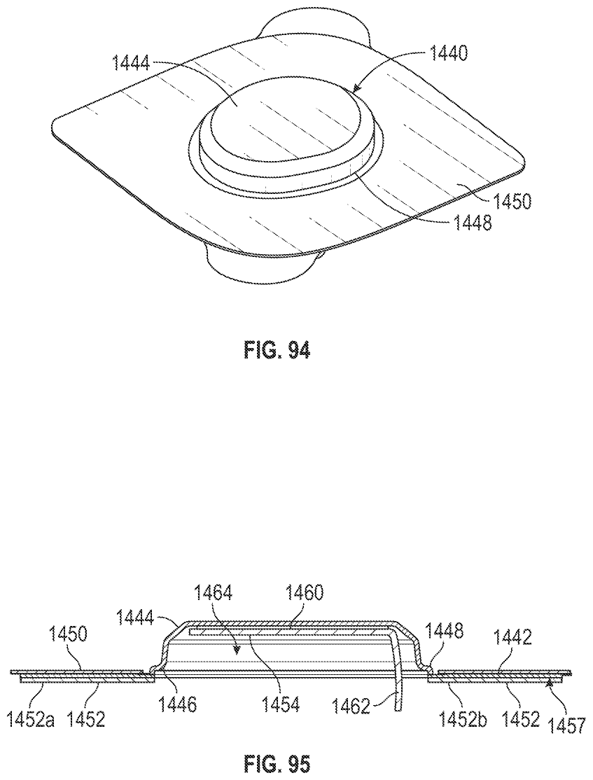

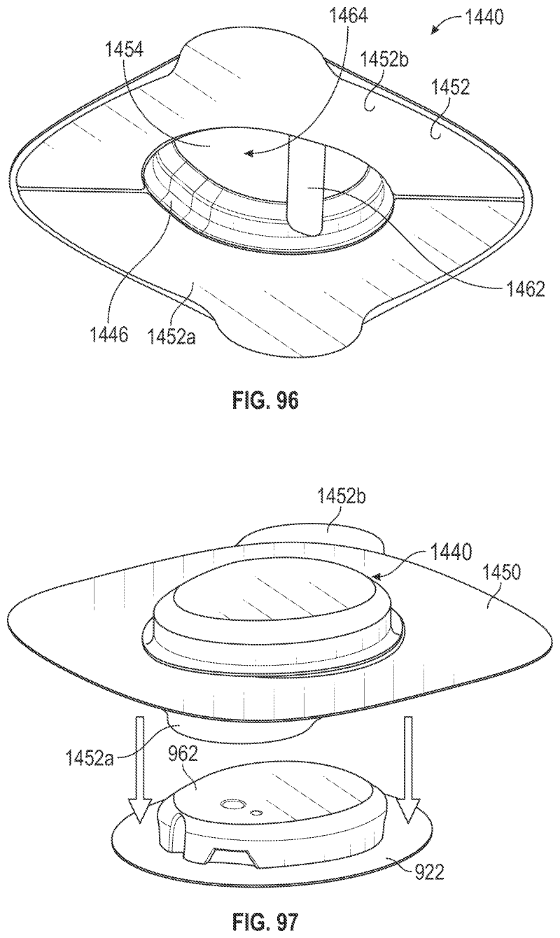

FIG. 94, for example, illustrates a perspective view of an applicator 1440 for supporting an overlay patch 1442 and configured to apply the overlay patch 1442 onto at least a portion of an on-skin wearable medical device when the on-skin wearable medical device is disposed on a skin of the host. FIG. 95 illustrates a cross sectional view of the applicator 1440. FIG. 96 illustrates a distal perspective view of the applicator 1440.

The applicator 1440 may be bonded to the overlay patch 1442 (as shown in the cross-sectional view of FIG. 95). The applicator 1440 may be non-separable from the overlay patch 1442. The applicator 1440 may be configured to be dispensed onto the on-skin wearable device upon application of the patch 1442.

The applicator 1440, for example, may comprise a body in the form of an alignment body 1444 for aligning the patch 1442 with the on-skin wearable medical device. The alignment body 1444 may include a central opening 1446 for receiving a portion of the on-skin wearable medical device for alignment with the on-skin wearable medical device.

The alignment body 1444 may comprise a shell that is configured to overlay and remain positioned upon the housing of the on-skin wearable medical device. In examples, the shell may be contoured to the shape of the housing of the on-skin wearable medical device. As such, the shell may be shaped to align with the housing of the on-skin wearable medical device in a defined rotational orientation upon positioning upon the housing of the on-skin wearable medical device. Such a feature may provide for the desired alignment of the patch 1442 relative to the on-skin wearable medical device. For example, if the housing of the on-skin wearable medical device has an oblong shape, the alignment body 1444 may have a corresponding oblong shape such that a rotational orientation of the alignment body 1444 is set upon application to the on-skin wearable medical device.

The shell may comprise a molded shell in examples, which may be contoured to the shape of the housing of the on-skin wearable medical device. The shell may have other constructions or shapes in examples. The shell may comprise a thin material such as thermoplastic polyurethane (TPU) or other forms of thermoplastics or other materials in examples. The shell may bound a cavity 1464 that is adapted to house the housing of the on-skin wearable medical device therein.

The alignment body 1444 may have an outer edge 1448 that may bond to the patch 1442. The alignment body 1444 may bond with the patch 1442 via welding, thermal bonding, mechanical joints, or use of adhesives, among other methods. The patch 1442 may extend radially outward from the alignment body 1444. The patch 1442 may form a ring extending around the alignment body 1444 in examples.

The applicator 1440 may include a backing layer 1450 and one or more liners 1452, 1454. The backing layer 1450 may be positioned upon an upper or proximal surface 1456 (marked in FIG. 99) of the patch 1442 in examples. In examples, the use of the backing layer 1450 may be excluded. The liner 1452 may be positioned upon and cover the lower, distal, or engaging surface 1457 of the patch 1442. The liner 1452 may include one or more liner sections 1452a, b as desired.

The alignment body 1444 may include an adhesive or adhesive layer 1460 in examples. The liner 1454 may cover the adhesive layer 1460 in examples. The liner 1454 may include a removal portion 1462 that may comprise a tab or wing that extends from the liner 1454. FIG. 96, for example, illustrates a distal perspective view of the applicator 1440 showing the position of the liners 1452, 1454.

The adhesive or adhesive layer 1460 may be positioned within a cavity 1464 for receiving the housing of the on-skin wearable medical device. The adhesive layer 1460 may be configured to adhere with an upper or proximal surface of the housing of the on-skin wearable medical device.

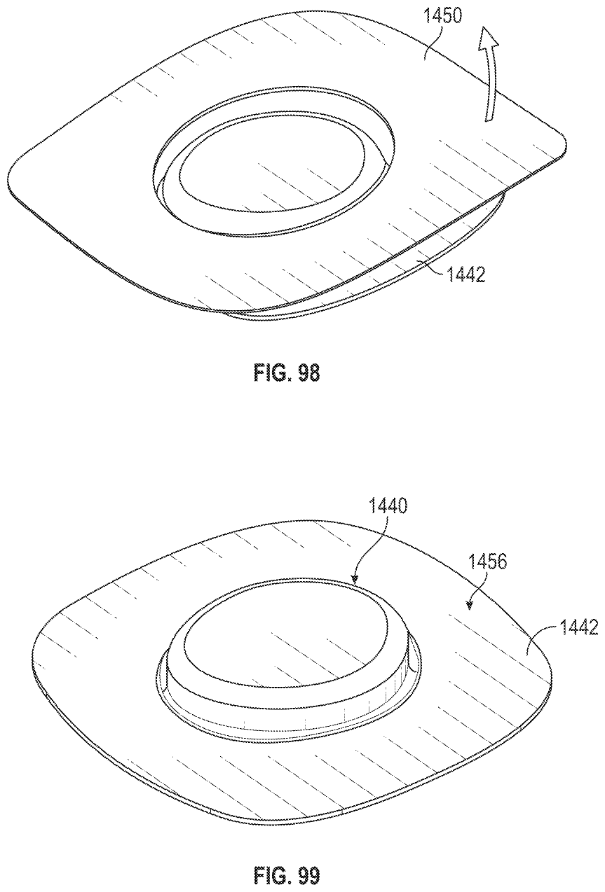

FIGS. 97 and 98 illustrate an exemplary deployment of the overlay patch 1442.

Prior to application, the liner 1454 may be removed by pulling on the removal portion 1462. As such, the adhesive layer 1460 with the cavity 1464 may be exposed for coupling with the housing 962 of the on-skin wearable medical device. The applicator 1440 may approach the on-skin wearable medical device with the liner sections 1452a, b in position upon the engaging surface 1457 of the overlay patch 1442 as represented in FIG. 97. In examples, the liner sections 1452a, b may be removed prior to positioning of the applicator 1440 onto the on-skin wearable medical device.

The applicator 1440 may be placed upon the housing 962 of the on-skin wearable medical device. The adhesive layer 1460 may couple to the housing 962 of the on-skin wearable medical device. Such a feature may provide stability for the process of removing the liner sections 1452a, b. The liner sections 1452a, b may be removed with the applicator 1440 positioned upon the housing 962 of the on-skin wearable medical device. The engaging surface 1457 of the overlay patch 1442 accordingly may adhere to the upper or proximal surfaces of the patch 922 of the on-skin wearable medical device, preferably without contacting the housing 962 of the on-skin wearable medical device.

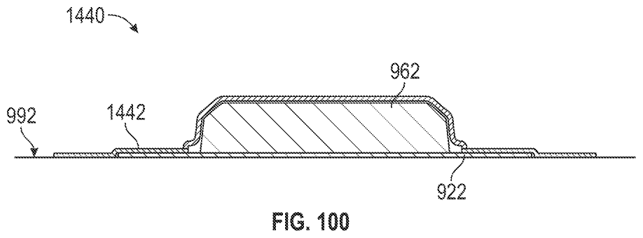

Referring to FIG. 98, the backing layer 1450 may be removed. The applicator 1440 may remain dispensed upon the on-skin wearable medical device as represented in FIG. 99. A user may manually smooth and press the overlay patch 1442 onto the patch 922 of the on-skin wearable medical device and/or the skin 992 of the host. The pressing and smoothing steps may be performed before and/or after removal of the backing layer 1450. A cross sectional view of a resulting configuration is shown in FIG. 100.

The applicator 1440 may remain upon the on-skin wearable medical device and may be worn by the host. The applicator 1440 may cover and protect at least the housing 962 of the on-skin wearable medical device from damage and/or ingress of undesired substances such as moisture. The applicator 1440 and patch 1442 may be removed by being peeled off of the deployment site at a desired time.

The features of the examples of FIGS. 94-100 may be utilized solely or in combination with any other example disclosed herein.

Various other configurations of applicators may be utilized in examples.

In examples, an on-skin wearable medical device may exclude the use of an underlay patch or may exclude the use of an underlay patch that extends radially outward from a housing. In such a configuration an applicator as disclosed herein may yet be utilized to apply an overlay patch to at least a portion of such an on-skin wearable medical device, including a housing of such a medical device. Applicators as disclosed herein may be utilized to apply an overlay patch to at least a portion of an on-skin wearable medical device that lacks an underlay patch or a housing in examples.

Examples of applicators or substrates as disclosed herein may be made from a variety of materials as desired. For example, applicators or substrates may be made from one or more of a polymer (e.g., a plastic or other form of polymer), a metal, or a paper-based material (e.g., cardboard, cardstock, or fibrous composites). Other forms of material may be utilized as desired. Flexible materials may be utilized as desired.

Examples of applicators as disclosed herein may beneficially improve the case upon which an overlay patch may be applied to a desired application site. Applicators as disclosed herein may be utilized for deployment of other forms of patches or other devices than disclosed herein.

Features of examples disclosed herein may be utilized solely or in combination with any other system, apparatus, or method disclosed herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular examples disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the examples.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to examples containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples described herein, but rather to also cover all modification and alternatives coming with the true scope and spirit of the invention.

1. A system for applying a patch onto at least a portion of an on-skin wearable medical device of a host, the system comprising:
an applicator for supporting the patch and configured to apply the patch onto at least the portion of the on-skin wearable medical device when the on-skin wearable medical device is disposed on a skin of the host.

2. The system of claim 1, wherein the applicator includes a substrate for supporting the patch.

3. The system of claim 2, wherein the substrate comprises a planar surface for the patch to be positioned upon and extend parallel with the planar surface.

4. (canceled)

5. (canceled)

6. The system of claim 1, wherein the applicator includes an alignment body configured to align the patch with the on-skin wearable medical device.

7. The system of claim 6, wherein the alignment body comprises a protrusion configured to extend towards the skin of the host upon the applicator applying the patch onto at least the portion of the on-skin wearable medical device.

8. The system of claim 7, wherein the protrusion includes a central opening configured to receive a portion of the on-skin wearable medical device.

9.-16. (canceled)

17. The system of claim 7, wherein the applicator includes a substrate for supporting the patch, and the applicator includes a plunger configured to be operated by a user to displace the protrusion relative to the substrate.

18.-22. (canceled)

23. The system of claim 1, wherein the applicator includes a first housing and a second housing, the first housing configured to slide relative to the second housing along an axis to apply the patch onto at least the portion of the on-skin wearable medical device, and the second housing including a cavity with a coil spring disposed therein, the coil spring biasing the first housing relative to the second housing and extending circumferentially about the axis.

24.-31. (canceled)

32. The system of claim 6, wherein the alignment body has a leading edge, and the applicator includes a substrate for supporting the patch, the substrate being positioned at the leading edge.

33. The system of claim 6, wherein the alignment body has a leading edge, and the applicator includes a substrate for supporting the patch, the substrate being spaced from the leading edge.

34.-70. (canceled)

71. A method comprising:
utilizing an applicator to apply a patch onto at least a portion of an on-skin wearable medical device when the on-skin wearable medical device is disposed on a skin of a host.

72. The method of claim 71, wherein the applicator includes a substrate for supporting the patch.

73. The method of claim 72, wherein the substrate comprises a planar surface for the patch to be positioned upon and extend parallel with the planar surface.

74. (canceled)

75. (canceled)

76. The method of claim 71, wherein the applicator includes an alignment body configured to align the patch with the on-skin wearable medical device.

77. The method of claim 76, wherein the alignment body comprises a protrusion configured to extend towards the skin of the host upon the applicator applying the patch onto at least the portion of the on-skin wearable medical device.

78. The method of claim 77, wherein the protrusion includes a central opening configured to receive a portion of the on-skin wearable medical device.

79.-86. (canceled)

87. The method of claim 77, wherein the applicator includes a substrate for supporting the patch, and the applicator includes a plunger configured to be operated by a user to displace the protrusion relative to the substrate.

88.-92. (canceled)

93. The method of claim 71, wherein the applicator includes a first housing and a second housing, the first housing configured to slide relative to the second housing along an axis to apply the patch onto at least the portion of the on-skin wearable medical device, and the second housing including a cavity with a coil spring disposed therein, the coil spring biasing the first housing relative to the second housing and extending circumferentially about the axis.

94.-101. (canceled)

102. The method of claim 76, wherein the alignment body has a leading edge, and the applicator includes a substrate for supporting the patch, the substrate being positioned at the leading edge.

103. The method of claim 76, wherein the alignment body has a leading edge, and the applicator includes a substrate for supporting the patch, the substrate being spaced from the leading edge.

104.-140. (canceled)
